(19) United States
(12) Patent Application Publication
(10) Pub. No.: US 2025/0000936 A1
(43) Pub. Date: Jan. 2, 2025

(54) TREATMENT OF GEOGRAPHIC ATROPHY

(71) Applicant: Apellis Pharmaceuticals, Inc., Waltham, MA (US)

(72) Inventors: Cedric Francois, Prospect, KY (US); Pascal Deschatelets, West Newton, MA (US); Federico Grossi, Needham, MA (US)

(21) Appl. No.: 18/690,607

(22) PCT Filed: Sep. 9, 2022
(86) PCT No.: PCT/US22/43127

§ 371 Date: Mar. 8, 2024

Related U.S. Application Data
(60) Provisional application No. 63/400,569, filed on Aug. 24, 2022.
(60) Provisional application No. 63/389,128, filed on Jul. 14, 2022.
(60) Provisional application No. 63/350,851, filed on Jun. 9, 2022.
(60) Provisional application No. 63/337,565, filed on May 2, 2022.
(60) Provisional application No. 63/320,460, filed on Mar. 16, 2022.
(60) Provisional application No. 63/254,348, filed on Oct. 11, 2021.
(60) Provisional application No. 63/250,800, filed on Sep. 30, 2021.
(60) Provisional application No. 63/242,475, filed on Sep. 9, 2021.

(51) Int. Cl. A61K 38/12; A61K 9/00; A61K 45/06; A61K 47/60; A61P 27/02

(52) U.S. Cl. CPC A61K 38/12; A61K 9/0048; A61K 45/06; A61K 47/60; A61P 27/02

(57) ABSTRACT

Methods and compositions for treatment of geographic atrophy are described.

### CROSS REFERENCE TO RELATED APPLICATIONS

The present application claims priority to U.S. Provisional Patent Application Nos. 63/242,475 filed Sep. 9, 2021, 63/250,800 filed Sep. 30, 2021, 63/254,348 filed Oct. 11, 2021, 63/320,460 filed Mar. 16, 2022, 63/337,565 filed May 2, 2022, 63/350,851 filed Jun. 9, 2022, 63/389,128 filed Jul. 14, 2022, and 63/400,569 filed Aug. 24, 2022, the entire contents of all of which are hereby incorporated by reference in their entirety.

### BACKGROUND

Geographic atrophy (GA) is an advanced form of age-related macular degeneration (AMD), a leading cause of blindness. GA lesions affect the central portion of the retina, known as the macula, which is responsible for central vision. GA is progressive and irreversible, leading to central visual impairment and permanent loss of vision. Based on published studies, approximately one million people have GA in the United States and five million people have GA globally (Rudnicka et al., Opththalmology 119:571-580 (2012); Wong et al., Lancet Glob Health 2: e106-116 (2014)). There are currently no approved treatments for GA.

### SUMMARY

In some aspects, the disclosure features a method of treating a subject suffering from or at risk of geographic atrophy (GA), e.g., GA secondary to age related macular degeneration (AMD), comprising intravitreally administering to an eye of the subject monthly (e.g., 30 days) or every other month (e.g., 60 days) a composition comprising pegcetacoplan, wherein: (a) the composition comprises about 15 mg/0.1 mL pegcetacoplan, and (b) following the start of the administering step (e.g., at about 12 months, at about 18 months, and/or at about 24 months, or after about 12 months, after about 18 months, and/or after about 24 months, following the start of the administering step), the composition reduces GA lesion growth in the eye by at least about 10% to at least about 25% (e.g., reduces GA lesion growth by at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%), relative to control.

In some embodiments, following the start of the administering step (e.g., at about 12 months or after about 12 months following the start of the administering step), the composition reduces GA lesion growth in the eye by at least about 16% to at least about 22%, relative to control. In some embodiments, following the start of the administering step (e.g., at about 12 months or after about 12 months following the start of the administering step), the composition reduces GA lesion growth in the eye by at least about 11% or at least about 12%, relative to control. In some embodiments, following the start of the administering step (e.g., at about 12 months or after about 12 months following the start of the administering step), the composition reduces GA lesion growth in the eye by at least about 14% to at least about 17%, relative to control. In some embodiments, following the start of the administering step (e.g., at about 12 months or after about 12 months following the start of the administering step), the composition reduces GA lesion growth in the eye by a statistically significant amount.

In some embodiments, following the start of the administering step (e.g., between about 18 months and about 24 months following the start of the administering step), the composition reduces GA lesion growth in the eye by at least about 25% to at least about 40%, relative to control. In some embodiments, following the start of the administering step (e.g., from about 18 months to about 24 months following the start of the administering step), the composition reduces GA lesion growth in the eye by at least about 29% or at least about 36%, relative to control. In some embodiments, following the start of the administering step (e.g., from about 18 months to about 24 months following the start of the administering step), the composition reduces GA lesion growth in the eye by at least about 29% to at least about 36%, relative to control. In some embodiments, following the start of the administering step (e.g., from about 18 months to about 24 months following the start of the administering step), the composition reduces GA lesion growth in the eye by a statistically significant amount. In some embodiments, following the start of the administering step (e.g., at between about 18 months and about 24 months following the start of the administering step), level of reduction in GA lesion growth in the eye is greater relative to level of reduction of GA legion growth in the eye at a time prior to about 18 months following the start of the administering step. In some embodiments, at a time between about 18 months and about 24 months following the start of the administering step, level of reduction in GA lesion growth in the eye is at least about 10%, 20%, 30%, 40%, 50%, or greater, relative to level of reduction of GA legion growth in the eye at a time prior to about 18 months following the start of the administering step. In some embodiments, rate of reduction in GA lesion growth in the eye between about 18 months and about 24 months following the start of the administering step is greater than (i) rate of reduction in GA lesion growth in the eye between the start of the administering step and about 6 months following the start of the administering step, and/or (ii) rate of reduction in GA lesion growth in the eye between the start of the administering step and about 12 months following the start of the administering step, and/or (iii) rate of reduction in GA lesion growth in the eye between the start of the administering step and about 18 months following the start of the administering step, and/or (iv) rate of reduction in GA lesion growth in the eye between about 6 months following the start of the administering step and about 12 months following the start of the administering step; and/or (v) rate of reduction in GA lesion growth in the eye between about 6 months following the start of the administering step and about 18 months following the start of the administering step; and/or (vi) rate of reduction in GA lesion growth in the eye between about 12 months following the start of the administering step and about 18 months following the start of the administering step.

In some embodiments, the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

In some embodiments, the composition comprises about 5% to about 6% trehalose.

In some embodiments, the composition is administered monthly (e.g., 30 days). In some embodiments, the composition is administered every other month (e.g., 60 days).

In some embodiments, the method further comprises administering an anti-VEGF agent to the subject (e.g., intravitreally administering an anti-VEGF agent to the same eye of the subject to which the composition was administered).

In some embodiments, the method further comprises diagnosing the subject as suffering from or at risk of geographic atrophy (GA), e.g., GA secondary to age related macular degeneration (AMD). In some embodiments, the diagnosing step comprises fundus autofluorescence (FAF) and/or optical coherence tomography (OCT). In some embodiments, the method further comprises selecting the subject for treatment if total GA area is ≥2.5 mm² (e.g., ≥2.5 mm² and ≤17.5 mm²).

In another aspect, the disclosure features a method of treating a subject suffering from or at risk of bilateral geographic atrophy (GA), e.g., GA secondary to age related macular degeneration (AMD), comprising intravitreally administering to a first eye (having or exhibiting GA) of the subject monthly (e.g., 30 days) or every other month (e.g., 60 days) a composition comprising pegcetacoplan, wherein: (a) the composition comprises about 15 mg/0.1 mL pegcetacoplan, and (b) following the start of the administering step (e.g., at about 12 months and/or at about 18 months, or after about 12 months and/or after about 18 months, following the start of the administering step), the composition reduces rate of GA lesion growth in the first eye by at least about 10% to at least about 20% (e.g., reduces rate of GA lesion growth by at least about 10%, at least about 11%, at least about 12%, at least about 13%, at least about 14%, at least about 15%, at least about 16%, at least about 17%, at least about 18%, at least about 19%, at least about 20%), relative to a second untreated eye (having or exhibiting GA) of the subject.

In some embodiments, following the start of the administering step (e.g., at about 12 months or after about 12 months following the start of the administering step), the composition reduces rate of GA lesion growth in the first eye by a statistically significant amount.

In some embodiments, the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

In some embodiments, the composition comprises about 5% to about 6% trehalose.

In some embodiments, the composition is administered monthly (e.g., 30 days). In some embodiments, the composition is administered every other month (e.g., 60 days).

In some embodiments, the method further comprises administering an anti-VEGF agent to the subject (e.g., intravitreally administering an anti-VEGF agent to the same eye of the subject to which the composition was administered).

In some embodiments, the method further comprises diagnosing the subject as suffering from or at risk of geographic atrophy (GA), e.g., GA secondary to age related macular degeneration (AMD). In some embodiments, the diagnosing step comprises fundus autofluorescence (FAF) and/or optical coherence tomography (OCT). In some embodiments, the method further comprises selecting the subject for treatment if total GA area is ≥2.5 mm² (e.g., ≥2.5 mm² and ≤17.5 mm²).

In another aspect, the disclosure features a method of treating a subject suffering from or at risk of geographic atrophy (GA), e.g., GA secondary to age related macular degeneration (AMD), and exhibiting extrafoveal lesions (e.g., solely extrafoveal lesions), the method comprising intravitreally administering to an eye of the subject (e.g., an eye exhibiting extrafoveal lesions, e.g., solely extrafoveal lesions) monthly or every other month a composition comprising pegcetacoplan, wherein: (a) the composition comprises about 15 mg/0.1 mL pegcetacoplan, and (b) following the start of the administering step (e.g., at about 12 months and/or at about 18 months, or after about 12 months and/or after about 18 months, following the start of the administering step), the composition reduces GA extrafoveal lesion growth in the eye by at least about 20% to at least about 30% (e.g., reduces GA extrafoveal lesion growth by at least about 20%, at least about 21%, at least about 22%, at least about 23%, at least about 24%, at least about 25%, at least about 26%, at least about 27%, at least about 28%, at least about 29%, or at least about 30%), relative to control.

In some embodiments, following the start of the administering step (e.g., at about 12 months or after about 12 months following the start of the administering step), the composition reduces GA extrafoveal lesion growth in the eye by a statistically significant amount.

In some embodiments, the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

In some embodiments, the composition comprises about 5% to about 6% trehalose.

In some embodiments, the composition is administered monthly (e.g., 30 days). In some embodiments, the composition is administered every other month (e.g., 60 days).

In some embodiments, the method further comprises administering an anti-VEGF agent to the subject (e.g., intravitreally administering an anti-VEGF agent to the same eye of the subject to which the composition was administered).

In some embodiments, the method further comprises diagnosing the subject as suffering from or at risk of geographic atrophy (GA), e.g., GA secondary to age related macular degeneration (AMD). In some embodiments, the diagnosing step comprises fundus autofluorescence (FAF) and/or optical coherence tomography (OCT). In some embodiments, the method further comprises selecting the subject for treatment if total GA area is ≥2.5 mm² (e.g., ≥2.5 mm² and ≤17.5 mm²).

In another aspect, the disclosure features a method of reducing GA lesion growth in an eye of a subject suffering from or at risk of geographic atrophy (GA), e.g., GA secondary to age related macular degeneration (AMD), to a target level, the method comprising intravitreally administering to the eye of the subject monthly (e.g., 30 days) or every other month (e.g., 60 days) a composition comprising pegcetacoplan, wherein: (a) the composition comprises about 15 mg/0.1 mL pegcetacoplan, and (b) the target level is a reduction in GA lesion growth in the eye by at least about 10% to at least about 25% (e.g., a reduction in GA lesion growth by at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%), relative to control, e.g., at about 12 months, at about 18 months, and/or at about 24 months, or after about 12 months, after about 18 months, and/or after about 24 months, following the start of the administering step.

In some embodiments, the target level is reduction of GA lesion growth in the eye by at least about 16% to at least about 22%, relative to control, e.g., at about 12 months following the start of the administering step. In some embodiments, the target level is reduction of GA lesion growth in the eye by at least about 11% or at least about 12%, relative to control, e.g., at about 12 months following the start of the administering step. In some embodiments, the target level is reduction of GA lesion growth in the eye by at least about 14% to at least about 17%, relative to control, e.g., at about 12 months or after about 12 months following the start of the administering step. In some embodiments, the target level is reduction of GA lesion growth in the eye by at least about 16% to at least about 19%, relative to control, e.g., at about 24 months or after about 24 months following the start of the administering step. In some embodiments, the target level is reduction of GA lesion growth in the eye by at least about 25% to at least about 40% (e.g., by at least about 29% to at least about 36%, e.g., by about 29%, e.g., by about 36%), relative to control, e.g., at a time from about 18 months to about 24 months following the start of the administering step. In some embodiments, the target level is a level of reduction of GA lesion growth at a time between about 18 months and about 24 months following the start of the administering step, which is at least about 10%, 20%, 30%, 40%, 50%, or greater, relative to level of reduction of GA legion growth in the eye at a time prior to about 18 months following the start of the administering step. In some embodiments, the target level is a level of reduction of GA lesion growth at a time between about 18 months and about 24 months following the start of the administering step, which is at least about 10%, 20%, 30%, 40%, 50%, or greater, relative to level of reduction of GA legion growth in the eye at 18 months following the start of the administering step. In some embodiments, the target level is a rate of reduction in GA lesion growth in the eye between about 18 months and about 24 months following the start of the administering step that is at least about 10%, 20%, 30%, 40%, 50%, or greater than (i) rate of reduction in GA lesion growth in the eye between the start of the administering step and about 6 months following the start of the administering step, and/or (ii) rate of reduction in GA lesion growth in the eye between the start of the administering step and about 12 months following the start of the administering step, and/or (iii) rate of reduction in GA lesion growth in the eye between the start of the administering step and about 18 months following the start of the administering step, and/or (iv) rate of reduction in GA lesion growth in the eye between about 6 months following the start of the administering step and about 12 months following the start of the administering step; and/or (v) rate of reduction in GA lesion growth in the eye between about 6 months following the start of the administering step and about 18 months following the start of the administering step; and/or (vi) rate of reduction in GA lesion growth in the eye between about 12 months following the start of the administering step and about 18 months following the start of the administering step.

In some embodiments, the target level is a statistically significant level of reduction of GA lesion growth in the eye, e.g., about 12 months, about 18 months, and/or about 24 months, or after about 12 months, after about 18 months, and/or after about 24 months, and/or between about 18 months and about 24 months, following the start of the administering step.

In some embodiments, the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

In some embodiments, the composition comprises about 5% to about 6% trehalose.

In some embodiments, the composition is administered monthly (e.g., 30 days). In some embodiments, the composition is administered every other month (e.g., 60 days).

In some embodiments, the method further comprises administering an anti-VEGF agent to the subject (e.g., intravitreally administering an anti-VEGF agent to the same eye of the subject to which the composition was administered).

In some embodiments, the method further comprises diagnosing the subject as suffering from or at risk of geographic atrophy (GA), e.g., GA secondary to age related macular degeneration (AMD). In some embodiments, the diagnosing step comprises fundus autofluorescence (FAF) and/or optical coherence tomography (OCT). In some embodiments, the method further comprises selecting the subject for treatment if total GA area is ≥2.5 mm² (e.g., ≥2.5 mm² and ≤17.5 mm²).

In another aspect, the disclosure features a method of reducing rate of GA lesion growth in a first eye of a subject suffering from or at risk of bilateral geographic atrophy (GA), e.g., GA secondary to age related macular degeneration (AMD), to a target level, the method comprising intravitreally administering to the first eye (having or exhibiting GA) of the subject monthly (e.g., 30 days) or every other month (e.g., 60 days) a composition comprising pegcetacoplan, wherein: (a) the composition comprises about 15 mg/0.1 mL pegcetacoplan, and (b) at about the target level is reduction of rate of GA lesion growth in the first eye by at least about 10% to at least about 20% (e.g., reduction of rate of GA lesion growth by at least about 10%, at least about 11%, at least about 12%, at least about 13%, at least about 14%, at least about 15%, at least about 16%, at least about 17%, at least about 18%, at least about 19%, at least about 20%), relative to a second untreated eye (having or exhibiting GA) of the subject, e.g., at about 12 months and/or at about 18 months, or after about 12 months and/or after about 18 months, following the start of the administering step.

In some embodiments, the target level is a statistically significant level of reduction of rate of GA lesion growth in the first eye, e.g., about 12 months or after about 12 months following the start of the administering step.

In some embodiments, the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

In some embodiments, the composition comprises about 5% to about 6% trehalose.

In some embodiments, the composition is administered monthly (e.g., 30 days). In some embodiments, the composition is administered every other month (e.g., 60 days).

In some embodiments, the method further comprises administering an anti-VEGF agent to the subject (e.g., intravitreally administering an anti-VEGF agent to the same eye of the subject to which the composition was administered).

In some embodiments, the method further comprises diagnosing the subject as suffering from or at risk of geographic atrophy (GA), e.g., GA secondary to age related macular degeneration (AMD). In some embodiments, the diagnosing step comprises fundus autofluorescence (FAF) and/or optical coherence tomography (OCT). In some embodiments, the method further comprises selecting the subject for treatment if total GA area is ≥2.5 mm² (e.g., ≥2.5 mm² and ≤17.5 mm²).

In another aspect, the disclosure features a method of reducing GA extrafoveal lesion growth in an eye (exhibiting extrafoveal lesions, e.g., solely extrafoveal lesions) of a subject suffering from or at risk of geographic atrophy (GA), e.g., GA secondary to age related macular degeneration (AMD), to a target level, the method comprising intravitreally administering to the eye of the subject monthly (e.g., 30 days) or every other month (e.g., 60 days) a composition comprising pegcetacoplan, wherein: (a) the composition comprises about 15 mg/0.1 mL pegcetacoplan, and (b) the target level is a reduction of GA extrafoveal lesion growth in the eye by at least about 20% to at least about 30% (e.g., reduction of GA extrafoveal lesion growth by at least about 20%, at least about 21%, at least about 22%, at least about 23%, at least about 24%, at least about 25%, at least about 26%, at least about 27%, at least about 28%, at least about 29%, or at least about 30%), relative to control, e.g., at about 12 months and/or at about 18 months, or after about 12 months and/or after about 18 months, following the start of the administering step. In some embodiments, the target level is (i) a reduction of GA extrafoveal lesion growth in the eye by at least about 25% to at least about 35% (e.g., reduction of GA extrafoveal lesion growth by at least about 25%, at least about 26%, at least about 27%, at least about 28%, at least about 29%, at least about 30%, at least about 31%, at least about 32%, at least about 33%, at least about 34%, or at least about 35%), relative to control, e.g., at about 24 months and/or after about 24 months, and/or between about 18 months and about 24 months, following the start of the administering step; and/or (ii) reduction of GA foveal lesion growth in the eye by at least about 25% to at least about 35% (e.g., reduction of GA foveal lesion growth by at least about 25%, at least about 26%, at least about 27%, at least about 28%, at least about 29%, at least about 30%, at least about 31%, at least about 32%, at least about 33%, at least about 34%, or at least about 35%), relative to control, e.g., at about 24 months and/or after about 24 months, and/or between about 18 months and about 24 months, following the start of the administering step.

In some embodiments, the target level is a statistically significant level of reduction of GA extrafoveal lesion growth in the eye, e.g., about 12 months or after about 12 months following the start of the administering step.

In some embodiments, the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

In some embodiments, the composition comprises about 5% to about 6% trehalose.

In some embodiments, the composition is administered monthly (e.g., 30 days). In some embodiments, the composition is administered every other month (e.g., 60 days).

In some embodiments, the method further comprises administering an anti-VEGF agent to the subject (e.g., intravitreally administering an anti-VEGF agent to the same eye of the subject to which the composition was administered).

In some embodiments, the method further comprises diagnosing the subject as suffering from or at risk of geographic atrophy (GA), e.g., GA secondary to age related macular degeneration (AMD). In some embodiments, the diagnosing step comprises fundus autofluorescence (FAF) and/or optical coherence tomography (OCT). In some embodiments, the method further comprises selecting the subject for treatment if total GA area is ≥2.5 mm² (e.g., ≥2.5 mm² and ≤17.5 mm²).

In another aspect, the disclosure features a composition (e.g., an aqueous composition) comprising about 15 mg/0.1 mL pegcetacoplan and about 5% to about 5.5% trehalose. In some embodiments, the composition comprises about 16-22 mM acetate buffer (e.g., about 16 mM, about 17 mM, about 18 mM, about 19 mM, about 20 mM, about 21 mM, or about 22 mM acetate buffer). In some embodiments, the composition has a pH of about 5.0. In some embodiments, the composition is sterile and preservative-free.

In another aspect, the disclosure features a composition (e.g., an aqueous composition) comprising about 15 mg/0.1 mL pegcetacoplan; about 5% to about 5.5% trehalose; about 16-22 mM acetate buffer (e.g., about 16 mM, about 17 mM, about 18 mM, about 19 mM, about 20 mM, about 21 mM, or about 22 mM acetate buffer); has a pH of about 5.0; is sterile and preservative-free.

In another aspect, the disclosure features a method of treating a subject suffering from or at risk of geographic atrophy (GA), e.g., GA secondary to age related macular degeneration (AMD), the method comprising intravitreally administering to an eye of the subject monthly (e.g., 30 days) or every other month (e.g., 60 days) a composition (e.g., an aqueous composition) comprising about 15 mg/0.1 mL pegcetacoplan; about 5% to about 5.5% trehalose; about 16-22 mM acetate buffer (e.g., about 16 mM, about 17 mM, about 18 mM, about 19 mM, about 20 mM, about 21 mM, or about 22 mM acetate buffer); has a pH of about 5.0; is sterile and preservative-free, thereby treating the GA.

### BRIEF DESCRIPTION OF THE DRAWING

FIG. 1 shows the structure of pegcetacoplan (“APL-2”), assuming n of about 800 to about 1100 and a PEG of about 40 kD.

FIG. 2 is a schematic of the DERBY and OAKS Phase 3 study design of intravitreal pegcetacoplan in patients with GA.

FIG. 3 shows LS mean change (±SE) from baseline in GA lesion (mm²) in the study eye over 12 months from the OAKS study. Least square (LS) means estimated from a mixed-effects model for repeated measures (MMRM). The mITT population was used for the analysis, defined as all randomized patients who received at least 1 injection of pegcetacoplan or sham and have baseline and at least one post-baseline value of GA lesion area in the study eye.

FIG. 4 shows LS mean change (±SE) from baseline in GA lesion (mm²) in the study eye over 12 months from the DERBY study. LS means estimated from a mixed-effects model for repeated measures. The mITT population was used for the analysis.

FIG. 5 shows LS mean change (±SE) from baseline in GA lesion (mm²) in the study eye over 12 months from the OAKS and DERBY studies combined. LS means estimated from a mixed-effects model for repeated measures. The mITT population was used for the analysis.

FIG. 6A shows LS mean change (±SE) from baseline in GA lesion (mm²) in the study eye over 12 months from the OAKS and DERBY studies from patients in United States sites. LS means estimated from a mixed-effects model for repeated measures. The mITT population was used for the analysis.

FIG. 6B shows LS mean change (±SE) from baseline in GA lesion (mm²) in the study eye over 12 months from the OAKS and DERBY studies from patients in sites from the rest of the world. LS means estimated from a mixed-effects model for repeated measures. The mITT population was used for the analysis.

FIG. 7A shows LS mean change (±SE) from baseline in GA lesion (mm²) in the study eye and in the fellow eye in sham pooled groups from the OAKS, DERBY, and FILLY studies. LS means estimated from a mixed-effects model for repeated measures. The mITT population was used for the analysis.

FIG. 7B shows mean change (±SE) from baseline in GA lesion (mm²) in the study eye and in the fellow eye from the OAKS, DERBY, and FILLY studies for patients treated every other month. LS means estimated from a mixed-effects model for repeated measures. The mITT population was used for the analysis.

FIG. 7C mean change (±SE) from baseline in GA lesion (mm²) in the study eye and in the fellow eye from the OAKS, DERBY, and FILLY studies for patients treated monthly. LS means estimated from a mixed-effects model for repeated measures. The mITT population was used for the analysis.

FIG. 7D shows LS mean change (±SE) from baseline in GA lesion (mm²) in the study eye and in the fellow eye from the OAKS and DERBY studies combined. Study eye vs. fellow eye comparison was prespecified; statistical modeling was performed post-hoc. LS means estimated from a mixed-effects model for repeated measures. The mITT population was used for the analysis. In addition, patients must have bilateral GA and a fellow eye that meets the following key characteristics at baseline: absence of CNV in the medical history; baseline GA lesion size between 2.5 and 17.5 mm² and have at least one study eye or fellow eye at measurement at Month 6 or Month 12.

FIG. 7E shows mean change from baseline in GA lesion (mm²) in the fellow eye over 12 months for the DERBY and the OAKS studies. Means obtained based on observed data. The mITT population was used for the analysis. In addition, patients must have bilateral GA and a fellow eye that meets the following key characteristics at baseline: absence of CNV in the medical history; baseline GA lesion size between 2.5 and 17.5 mm².

FIG. 8A shows LS mean change (±SE) from baseline over 12 months in GA lesion (mm²) in the study eye in patients with extrafoveal GA lesions from the OAKS and DERBY studies. LS means estimated from a mixed-effects model for repeated measures. The mITT population was used for the analysis.

FIG. 8B shows LS mean change (±SE) from baseline over 12 months in GA lesion (mm²) in the study eye in patients with extrafoveal GA lesions from the OAKS and DERBY studies combined. LS means estimated from a mixed-effects model for repeated measures. The mITT population was used for the analysis.

FIG. 8C shows LS mean change (±SE) from baseline over 12 months in GA lesion (mm²) in the study eye in patients with subfoveal GA lesions from the OAKS and DERBY studies. LS means estimated from a mixed-effects model for repeated measures. The mITT population was used for the analysis.

FIG. 8D shows LS mean change (±SE) from baseline over 12 months in GA lesion (mm²) in the study eye in patients with subfoveal GA lesions from the OAKS and DERBY studies combined. LS means estimated from a mixed-effects model for repeated measures. The mITT population was used for the analysis.

FIG. 9 shows LS mean change (±SE) from baseline in GA lesion (mm²) in the study eye over 12 months from the OAKS, DERBY, and FILLY studies from covariate-adjusted analyses. LS means estimated from a mixed-effects model for repeated measures. The mITT population was used for the analysis.

FIG. 10 shows safety profile from the OAKS and DERBY studies combined.

FIG. 11 shows LS mean change (±SE) from baseline in GA lesion (mm²) in the study eye over 18 months from the DERBY and OAKS studies. Least square (LS) means estimated from a mixed-effects model for repeated measures (MMRM). The mITT population was used for the analysis, defined as all randomized patients who received at least 1 injection of pegcetacoplan or sham and have baseline and at least one post-baseline value of GA lesion area in the study eye.

FIG. 12A shows lesion growth percent reduction vs. sham pooled for the DERBY and OAKS studies. Percent reduction vs. sham for Month 0 to Month 18 was estimated from a piecewise linear slope model with 6-months segments.

FIG. 12B shows lesion growth percent reduction vs. sham pooled from the OAKS and DERBY studies combined. Percent reduction vs. sham for Month 0 to Month 18 was estimated from a piecewise linear slope model with 6-months segments.

FIG. 13 shows LS mean change (±SE) from baseline in GA lesion (mm²) of the study eye with MMRM model assuming a piecewise linear trend in time with knots at month 6 and month 12 for the DERBY and OAKS studies.

FIG. 14 shows LS mean change (±SE) from baseline in GA lesion (mm²) in the study eye of patients with extrafoveal lesions over 18 months from the OAKS and DERBY studies. LS means estimated from a mixed-effects model for repeated measures. The mITT population was used for the analysis. Extrafoveal was defined as lesion with distance >0 to foveal center point.

FIG. 15 shows LS mean change (±SE) from baseline in GA lesion (mm²) in the study eye of patients with foveal lesions over 18 months from the OAKS and DERBY studies. LS means estimated from a mixed-effects model for repeated measures. The mITT population was used for the analysis. Foveal was defined as lesion edge within center point of the fovea.

FIG. 16 shows LS mean change (±SE) from baseline in GA lesion (mm²) in the study eye of patients with foveal or extrafoveal lesions from the OAKS and DERBY studies combined. LS means estimated from a mixed-effects model for repeated measures. The mITT population was used for the analysis. Foveal was defined as lesion edge within center point of the fovea.

FIG. 17 shows LS mean change (±SE) from baseline in GA lesion (mm²) in the study eye over 18 months from the OAKS and DERBY studies from covariate-adjusted analyses. LS means estimated from a mixed-effects model for repeated measures. The mITT population was used for the analysis.

FIG. 18 shows LS mean change (±SE) from baseline in GA lesion (mm²) in the study eye over 18 months from the OAKS and DERBY studies from covariate-adjusted analyses of patients with extrafoveal lesions. LS means estimated from a mixed-effects model for repeated measures. The mITT population was used for the analysis.

FIG. 19 shows LS mean change (±SE) from baseline in GA lesion (mm²) in the study eye over 18 months from the OAKS and DERBY studies from covariate-adjusted analyses of patients with foveal lesions. LS means estimated from a mixed-effects model for repeated measures. The mITT population was used for the analysis.

FIG. 20 shows LS mean change (±SE) from baseline in GA lesion (mm²) in the study eye and in the fellow eye from the DERBY study for patients treated monthly, every other month, or in sham pooled groups. LS means estimated from a mixed-effects model for repeated measures. The mITT population was used for the analysis. In addition, patients had bilateral GA with at least one post-baseline fundus autofluorsence assessment and with fellow eyes that met the following characteristics at baseline: absence of CNV in the medical history; baseline GA lesion size between 2.5 and 17.5 mm²; presence of any pattern of hyperautofluorescence in the junctional zone of GA; and GA not confluent with peripapillary atrophy.

FIG. 21 shows LS mean change (±SE) from baseline in GA lesion (mm²) in the study eye and in the fellow eye from the OAKS study for patients treated monthly, every other month, or in sham pooled groups. LS means estimated from a mixed-effects model for repeated measures. The mITT population was used for the analysis. In addition, patients had bilateral GA with at least one post-baseline fundus autofluorsence assessment and with fellow eyes that met the following characteristics at baseline: absence of CNV in the medical history; baseline GA lesion size between 2.5 and 17.5 mm²; presence of any pattern of hyperautofluorescence in the junctional zone of GA; and GA not confluent with peripapillary atrophy.

FIG. 22 is a schematic illustrating various subgroups assessed based on lesion distance from foveal center point.

FIG. 23A shows LS mean change (±SE) from baseline in GA lesion (mm²) in the study eye from the OAKS and DERBY studies in patients having a lesion distance to foveal center point in the study eye of >250 microns, and treated monthly, every other month, or in sham pooled groups. Foveal was defined as lesion edge within center point of the fovea. LS means estimated from a mixed-effects model for repeated measures. The modified intention-to-treat population was used for the analysis.

FIG. 23B shows LS mean change (±SE) from baseline in GA lesion (mm²) in the study eye from the OAKS and DERBY studies in patients having a lesion distance to foveal center point in the study eye of ≤250 microns, and treated monthly, every other month, or in sham pooled groups. Foveal was defined as lesion edge within center point of the fovea. LS means estimated from a mixed-effects model for repeated measures. The modified intention-to-treat population was used for the analysis.

FIG. 24A shows percentage reduction versus sham pooled in the study eye from OAKS in patients having a lesion distance to foveal center point of >50 microns, >100 microns, >150 microns, and >200 microns (as depicted in FIG. 22), and treated monthly or every other month.

FIG. 24B shows percentage reduction versus sham pooled in the study eye from DERBY in patients having a lesion distance to foveal center point of >50 microns, >100 microns, >150 microns, and >200 microns (as depicted in FIG. 22), and treated monthly or every other month.

FIG. 25A is a schematic representation of GA progression.

FIG. 25B describes methods and quartile definitions for post-hoc analysis of the DERBY and OAKS studies.

FIG. 26 shows distribution of patients from the DERBY and OAKS studies by study arm across quartiles.

FIG. 27 shows distribution of patients from the DERBY and OAKS studies by study arm across Q1 and Q4 quartiles.

FIG. 28 shows LS mean change (±SE) from baseline in GA lesion (mm²) in the study eye from baseline to month 24 from the DERBY and OAKS studies. Least square (LS) means estimated from a mixed-effects model for repeated measures (MMRM). The mITT population was used for the analysis, defined as all randomized patients who received at least 1 injection of pegcetacoplan or sham and have baseline and at least one post-baseline value of GA lesion area in the study eye.

FIG. 29 shows lesion growth percent reduction vs. sham pooled from the DERBY and the OAKS studies. Percent reduction vs. pooled sham for Month 0 to Month 24 was estimated from a piecewise linear slope model with 6-months segments. Point estimates for the month 0 to month 18 segments varied marginally from results described in Example 2 due to inclusion of month 20 to month 24 data in the statistical model.

FIG. 30 shows lesion growth percent reduction vs. sham pooled from the DERBY and OAKS studies combined. Percent reduction vs. pooled sham for Month 0 to Month 24 was estimated from a piecewise linear slope model with 6-months segments using the combined patient-level data, not a simple average of results, from the two studies. Point estimates for the month 0 to month 18 segments varied marginally from results described in Example 2 due to inclusion of month 20 to month 24 data in the statistical model.

FIG. 31 shows LS mean change (±SE) from baseline NL-BCVA Score (ETDRS Letters) from the DERBY and OAKS studies combined. Least square (LS) means estimated from a mixed effects model for repeated measures (MMRM). The mITT population was used for the analysis, defined as all randomized patients who received at least 1 injection of pegcetacoplan or sham and have baseline and at least one post baseline value of GA lesion area in the study eye.

FIG. 32 shows safety profile from the DERBY and OAKS studies combined.

### DETAILED DESCRIPTION OF CERTAIN EMBODIMENTS

### I. Definitions

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Antibody: As used herein, the term “antibody” refers to an immunoglobulin or a derivative thereof containing an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the disclosure the antibody is a fragment such as a Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16:535-539. The antibody may be partially or completely humanized. An antibody may be polyclonal or monoclonal, though for purposes of the present disclosure monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). In some embodiments, the term “about X” includes the number “X” and numbers that fall within a range of 5%, 10%, 15%, or 20% in either direction (greater than or less than) of the number X.

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered followed by one or more additional agents. In the separate administration protocol, two or more agents may be administered a few minutes apart, or a few hours apart, or a few days apart, or a few weeks apart. In some embodiments, two or more agents may be administered 1-2 weeks apart. In some embodiments, if two or more agents useful for treating the same disease are administered in combination, each of the two or more agents may be administered using a dosing regimen that would be used if such agent were being used as the sole agent for treating the disease.

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the disclosure the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual or subject who is “suffering from” a disease, disorder, and/or condition has been diagnosed with and/or displays one or more symptoms of a disease, disorder, and/or condition.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent can be an agent that, when administered to a subject, can prevent an undesired side effect, such as an immune response to a viral vector described herein. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., GA, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered prophylactically, i.e., before development of any symptom or manifestation of the condition. Typically in this case the subject will be at risk of developing the condition.

### II. Complement System

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. It will be understood that the names “C2a” and “C2b” were subsequently reversed in the scientific literature.

The alternative pathway is initiated by and amplified at, e.g., microbial surfaces and various complex polysaccharides. In this pathway, hydrolysis of C3 to C3 (H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b.

C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above. Further details are found, e.g., in Kuby Immunology, 6th ed., 2006; Paul, W. E., Fundamental Immunology, Lippincott Williams & Wilkins; 6th ed., 2008; and Walport M J., Complement. First of two parts. N Engl J Med., 344 (14): 1058-66, 2001.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b: C4b receptor), complement receptor type 2 (CR2), membrane cofactor protein (MCP; CD46), decay-accelerating factor (DAF, also known as CD55), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders.

### III. Compstatin Analogs

Methods of the disclosure include treatment of GA using compstatin analogs. Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7 (3): 619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32 (Pt 1): 28-32, 2004, Mallik, B., et al., J. Med. Chem., 274-286, 2005; Katragadda, M., et al. J. Med. Chem., 49:4616-4622, 2006; WO2007062249 (PCT/US2006/045539); WO2007044668 (PCT/US2006/039397), WO/2009/046198 (PCT/US2008/078593); WO/2010/127336 (PCT/US2010/033345) and discussion below.

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Table 1 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 1 are cyclized via a disulfide bond between the two Cys residues when used in the therapeutic compositions and methods of the disclosure. Alternate means for cyclizing the peptides are also within the scope of the disclosure.

TABLE 1

| | | SEQ | Activity |
|---|---|---|---|
| | | ID | over |
| Peptide | Sequence | NO: | compstatin |
| Compstatin | H- | 8 | * |
| | ICVVQDWGHHRCT- | | |
| | CONH2 | | |
| Ac- | Ac- | 9 | 3xmore |
| compstatin | ICVVQDWGHHRCT- | | |
| | CONH2 | | |
| Ac-V4Y/H9A | Ac- | 10 | 14xmore |
| | ICVYQDWGAHRCT- | | |
| | CONH2 | | |
| Ac-V4W/ | Ac- | 11 | 27xmore |
| H9A-OH | ICVWQDWGAHRCT- | | |
| | COOH | | |
| Ac-V4W/H9A | Ac- | 12 | 45xmore |
| | ICVWQDWGAHRCT- | | |
| | CONH2 | | |
| Ac-V4W/ | Ac- | 13 | 55xmore |
| H9A/T13dT- | ICVWQDWGAHRCdT- | | |
| OH | COOH | | |
| Ac-V4(2- | Ac-ICV(2-Nal) | 14 | 99xmore |
| Nal)/H9A | QDWGAHRCT-CONH2 | | |
| Ac V4(2- | Ac-ICV(2-Nal) | 15 | 38xmore |
| Nal)/H9A- | QDWGAHRCT-COOH | | |
| OH | | | |
| Ac V4(1- | Ac-ICV(1-Nal) | 16 | 30xmore |
| Nal)/H9A- | QDWGAHRCT-COOH | | |
| OH | | | |
| Ac- | Ac-ICV(2-Igl) | 17 | 39xmore |
| V42Igl/ | QDWGAHRCT- | | |
| H9A | CONH2 | | |
| Ac- | Ac-ICV(2-Igl) | 18 | 37xmore |
| V42Igl/ | QDWGAHRCT- | | |
| H9A-OH | COOH | | |
| Ac-V4Dht/ | Ac- | 19 | 5xmore |
| H9A-OH | ICVDhtQDWGA | | |
| | HRCT-COOH | | |
| Ac-V4 | Ac-ICV(Bpa) | 20 | 49xmore |
| (Bpa)/ | QDWGAHRCT- | | |
| H9A-OH | COOH | | |
| Ac-V4 | Ac-ICV(Bpa) | 21 | 86xmore |
| (Bpa)/H9A | QDWGAHRCT- | | |
| | CONH2 | | |
| Ac-V4 | Ac-ICV(Bta) | 22 | 65xmore |
| (Bta)/ | QDWGAHRCT- | | |
| H9A-OH | COOH | | |
| Ac-V4 | Ac-ICV(Bta) | 23 | 64xmore |
| (Bta)/H9A | QDWGAHRCT- | | |
| | CONH2 | | |
| Ac-V4W/ | Ac-ICVWQDWG | 24 | 64xmore |
| H9(2-Abu) | (2-Abu)HRCT- | | |
| | CONH2 | | |
| +G/V4W/H9A | H- | 25 | 38xmore |
| +AN-OH | GICVWQDWGAH | | |
| | RCTAN-COOH | | |
| Ac-V4 | Ac-ICV(5fW) | 26 | 31xmore |
| (5fW)/H9A | QDWGAHRCT- | | |
| | CONH₂ | | |
| Ac-V4(5- | Ac-ICV(5- | 27 | 67xmore |
| MeW)/H9A | methyl-W) | | |
| | QDWGAHRCT- | | |
| | CONH₂ | | |
| Ac-V4(1- | Ac-ICV(1- | 28 | 264xmore |
| MeW)/H9A | methyl-W) | | |
| | QDWGAHRCT- | | |
| | CONH₂ | | |
| Ac-V4W/W7 | Ac-ICVWQD | 29 | 121xmore |
| (5fW)/H9A | (5fW) | | |
| | GAHRCT- | | |
| | CONH₂ | | |
| Ac-V4 | Ac-ICV(5fW) | 30 | NA |
| (5fW)/W7 | QD(5fW) | | |
| (5fW)/H9A | GAHRCT- | | |
| | CONH₂ | | |
| Ac-V4(5- | Ac-ICV(5- | 31 | NA |
| MeW)/W7 | methyl-W)QD | | |
| (5fWV)H9A | (5fW)GAHRCT- | | |
| | CONH₂ | | |
| Ac-V4 | Ac-ICV(1- | 32 | 264xmore |
| (1MeW)/ | methyl-W)QD | | |
| W7(5fW)/ | (5fW)GAHRCT- | | |
| H9A | CONH₂ | | |
| +G/V4 | H-GICV(6fW) | 33 | 126xmore |
| (6fW)/W7 | QD(6fW) | | |
| (6fW)H9A | GAHRCTN- | | |
| +N-OH | COOH | | |
| Ac-V4(1- | Ac-ICV(1- | 34 | 264xmore |
| formyl- | formyl-W) | | |
| W)/H9A | QDWGAHRCT- | | |
| | CONH₂ | | |
| Ac-V4(5- | Ac-ICV(1- | 35 | 76xmore |
| methoxy- | methyoxy-W) | | |
| W)/H9A | QDWGAHRCT- | | |
| | CONH₂ | | |
| G/V4(5f- | H-GICV(5fW) | 36 | 112xmore |
| W)/W7 | QD(5fW) | | |
| (5fW)/H9A | GAHRCTN- | | |
| +N-OH | COOH | | |

NA = not available

In certain embodiments of the compositions and methods of the disclosure, the compstatin analog has a sequence selected from sequences 9-36. In some embodiments, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g., to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 9-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo (ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In some embodiments, the polymeric backbone or scaffold comprises multiple reactive functional groups, such as carboxylic acids, anhydride, or succinimide groups. The polymeric backbone or scaffold is reacted with the compstatin analogs. In some embodiments, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In some embodiments, short chains are prepolymerized, functionalized, and then a mixture of short chains of different composition are assembled into longer polymers.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In general and for compounds depicted herein, a polyethylene glycol moiety is drawn with the oxygen atom on the right side of the repeating unit or the left side of the repeating unit.

In cases where only one orientation is drawn, the present disclosure encompasses both orientations (i.e., (CH₂CH₂O)ₙ and (OCH₂CH₂)ₙ) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

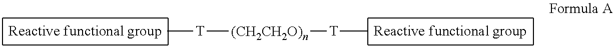

wherein each T and “Reactive functional group” is independently as defined below, and described in classes and subclasses herein, and n is as defined above and described in classes and subclasses herein.

Each T is independently a covalent bond or a C1-12 straight or branched, hydrocarbon chain wherein one or more carbon units of T are optionally and independently replaced by —O—, —S—, —N(R^{x})—, —C(O)—, —C(O)O—, —OC(O)—, —N(R^{x}) C(O)—, —C(O)N(R^{x})—, —S(O)—, —S(O)₂—, —N(R^{x})SO₂—, or —SO₂N(R^{x})—; and
each R^{x} is independently hydrogen or C1-6 aliphatic.

The Reactive functional group has the structure —COO—NHS.

Exemplary bifunctional PEGs of formula A include:

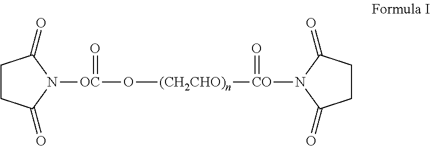

In some embodiments, a functional group (for example, an amine, hydroxyl, or thiol group) on a compstatin analog is reacted with a PEG-containing compound having a “reactive functional group” as described herein, to generate such conjugates. By way of example, Formula I can form compstatin analog conjugates having the structure:

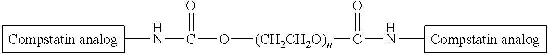

wherein,

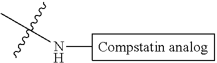 [SMILES: *NC]

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

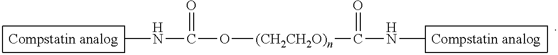

In some embodiments of formulae and embodiments described herein,

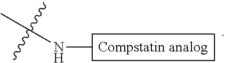 [SMILES: *NC]

represents point of attachment of a lysine side chain group in a compstatin analog having the structure:

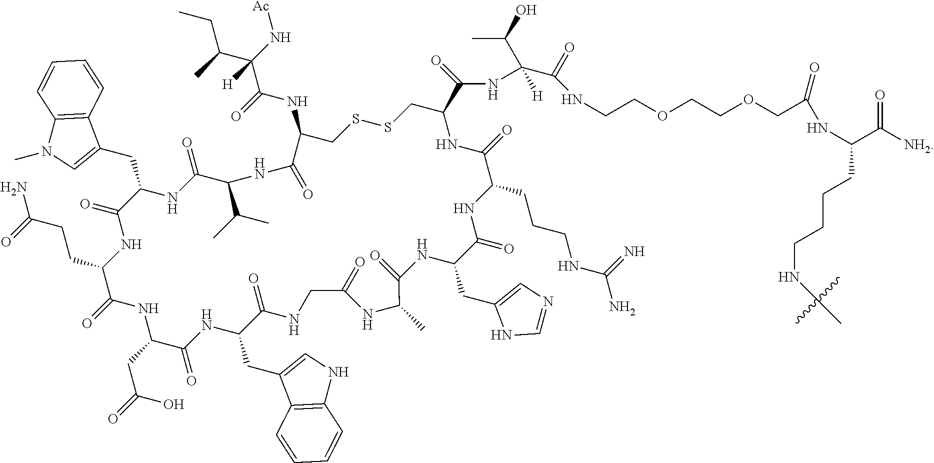 [SMILES: *NCCCC[C@H](NC(=O)COCCOCCNC(=O)[C@@H](NC(=O)[C@@H]1CSSC[C@H](NC(=O)[C@@H](NC(C)=O)[C@@H](C)CC)C(=O)N[C@@H](C(C)C)C(=O)N[C@@H](Cc2cn(C)c3ccccc23)C(=O)N[C@@H](CCC(N)=O)C(=O)N[C@@H](CC(=O)O)C(=O)N[C@@H](Cc2c[nH]c3ccccc23)C(=O)NCC(=O)N[C@@H](C)C(=O)N[C@@H](Cc2cnc[nH]2)C(=O)N[C@@H](CCCNC(=N)N)C(=O)N1)[C@@H](C)O)C(N)=O]

wherein the symbol “ [symbol] ” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)nCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the α-amino group of an amino acid residue. In some embodiments, an amine group is the amine group of the lysine side chain. In some embodiments, a compstatin analog is conjugated to a PEG moiety through the amino group of a lysine side chain (ε-amino group) via a linker having the structure of NH₂(CH₂CH₂O)nCH₂C(═O) OH, wherein n is 1 to 1000. In some embodiments, a compstatin analog is conjugated to the PEG moiety through the amino group of a lysine side chain via an AEEAc linker. In some embodiments, the NH₂(CH₂CH₂O)nCH₂C(═O) OH linker introduces a —NH(CH₂CH₂O)nCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the α-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the s-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

In certain embodiments a compstatin analog may be represented as M-AEEAc-Lys-B₂, wherein B₂ is a blocking moiety, e.g., NH₂, M represents any of SEQ ID NOs: 9-36, with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

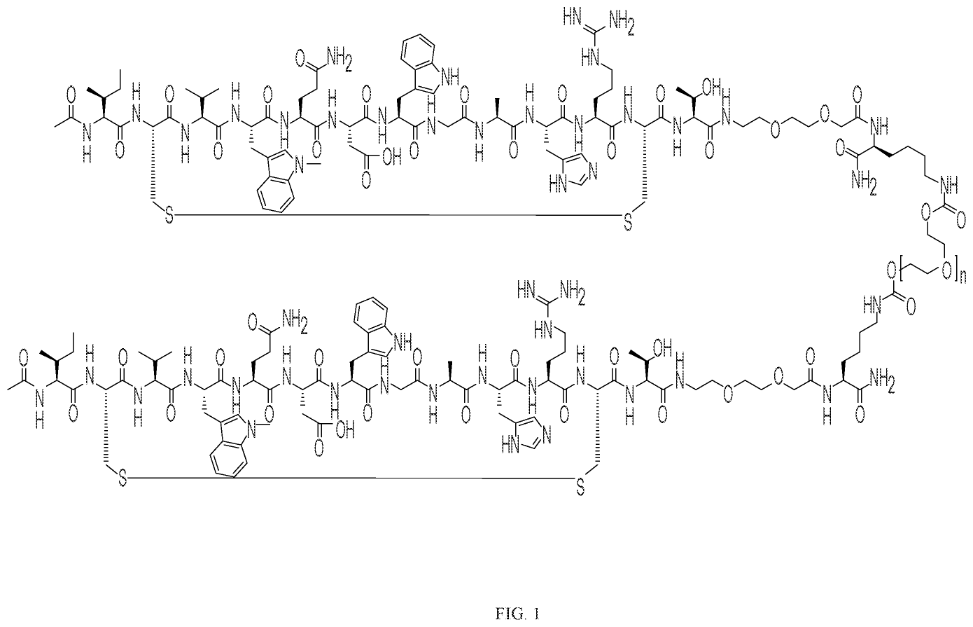

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted in FIG. 1. It will be understood that the PEG moiety may have a variety of different molecular weights or average molecular weights in various embodiments, as described herein. In certain embodiments, a compstatin analog is pegcetacoplan (“APL-2”), having the structure of the compound of FIG. 1 with n of about 800 to about 1100 and a PEG having an average molecular weight of about 40 kD. Pegcetacoplan is also referred to as Poly(oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy) ethoxy]acetyl-N⁶-carboxy-L-lysinamide cyclic (2->12)-(disulfide); or O,O′-bis[(S²,S¹²-cyclo{N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy) ethoxy]acetyl-L-lysinamide})-N^{6,15}-carbonyl]polyethylene glycol (n=800-1100). Additional compstatin analogs are described in, e.g., WO 2012/155107 and WO 2014/078731.

### IV. Complement-Mediated Eye Disorders

In some embodiments, a compstatin analog described herein is introduced into the eye of a subject for treatment of an eye disorder such as age-related macular degeneration (AMD). For example, a compstatin analog may be introduced into the vitreous cavity (e.g., by intravitreal injection), for treatment of a subject suffering from or at risk of AMD. In some embodiments the AMD is neovascular (wet or exudative) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with a compstatin analog as compared with control (e.g., patients given a sham injection). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD. In some embodiments, a subject has one eye with GA and one eye without GA. In some embodiments, a subject has one eye with GA and one eye without wet AMD. In some embodiments, a subject has one eye with GA and one eye with wet AMD. In some embodiments, a subject has one eye having both GA and neovascular AMD and one eye without wet AMD. In some embodiments, a subject has one eye having both GA and neovascular AMD and one eye with wet AMD. In some embodiments, both eyes of a subject have both GA and neovascular AMD.

Geographic atrophy (GA) is a late-stage disease manifestation in nonneovascular age-related macular degeneration (AMD) that progresses to severe central vision loss. Geographic atrophy has traditionally been defined on color fundus photography (CFP) as a sharply delineated circular or oval area of hypopigmentation or depigmentation in which choroidal vessels are visible. The size requirement for GA varies with the different studies, ranging from one eighth to one fourth of a disc area (corresponding roughly to 175 mm and 430 mm in diameter, respectively) on CFP (see, e.g., Bird et al., Surv. Ophthalmol. 39:367-374 (1995) and Schmitz-Valckenberg, Ophthalmologica 237:11-20 (2017)). Geographic atrophy was retained as a term for a late stage of AMD in the 2013 Beckman CFP classification of AMD (see Ferris et al., Ophthalmology 120:844-851 (2013)). As a clinical trial end point, the ability to slow GA expansion using a novel therapy has been approved by regulatory authorities, in which atrophy can be defined and quantified by fundus autofluorescence (FAF) (Csaky et al., Invest. Ophthalmol. Vis. Sci. 58:3456-3463 (2017); Holz et al., Ophthalmology 121:1079-1091 (2014); Schmitz-Valckenberg et al., Ophthalmology 123:361-368 (2016)). Recent trials aiming to slow progression of atrophy were required to enroll eyes with established regions of GA that could be measured reliably by a reading center, usually measuring at least 0.5 to 1.0 disc areas (1.25-2.5 mm²) (Cheng et al., Ophthalmol. Retina 2:518-525 (2018); Holz et al., JAMA Ophthalmol. 136:666-677 (2018); Rosenfeld et al., Ophthalmology 125:1556-1567 (2018)). In some embodiments, GA is assessed using FAF. In some embodiments, GA is assessed using optical coherence tomography (OCT).

Drusen are localized extracellular deposits of lipoproteinaceous material that accumulate between the retinal pigment epithelium (RPE) and the capillary network in the choroid (choriocapillaris), typically between the RPE and Bruch's membrane (a multilayered extracellular matrix complex that separates the RPE from the choriocapillaris). It will be appreciated that the term “druse” is sometimes used in the art to refer to a single such deposit (i.e., as a singular referent) while “drusen” is sometimes used in the art to refer to multiple such deposits (i.e., as a plural referent). As used herein, the term “drusen” should be understood to encompass a single “druse” or multiple “drusen” in various embodiments unless indicated to the contrary or clearly evident from the context. Also, reference to “a druse” should be understood to encompass reference to a single “druse” or multiple “drusen” in various embodiments unless indicated to the contrary or clearly evident from the context. Drusen are a clinical hallmark of AMD and are typically the earliest clinical finding in AMD. The presence, location, size, and number of drusen are factors used in the art for classifying AMD into stages and monitoring its progression. However, a few small drusen are commonly observed in the eyes of people over 40 years of age, most of whom do not go on to develop AMD. Drusen can be detected, assessed, and/or classified according to known methods, e.g., imaging, scanning laser ophthalmoscopy, and optical coherence tomography (OCT) (e.g., spectral domain optical coherence tomography (SD-OCT)). Additional methods are described in, e.g., WO2014/028861.

In some embodiments early, intermediate, or advanced AMD are defined in accordance with the classification scheme used in the Age-Related Eye Diseases Study (AREDS) (The Age-Related Eye Disease Study Research Group. A randomized, placebo-controlled, clinical trial of high-dose supplementation with vitamins C and E, beta carotene, and zinc for age-related macular degeneration and vision loss: AREDS report number 8. Arch Ophthalmol 2001; 119:1417-36). The classification of AMD from the AREDS is summarized as follows:

No AMD (AREDS category 1) is characterized by no or few small drusen (<63 microns in diameter).

Early AMD (AREDS category 2) is characterized by presence of a combination of multiple small drusen, few intermediate drusen (63 to 124 microns in diameter), or RPE abnormalities.

Intermediate AMD (AREDS category 3) is characterized by presence of extensive intermediate drusen, at least one large druse (at least 125 microns in diameter), or geographic atrophy not involving the center of the macula.

Advanced AMD (AREDS category 4) is characterized by geographic atrophy involving the center of the macula and/or neovascular macular degeneration. Neovascular macular degeneration is typically associated with manifestations of CNV and/or retinal or RPE detachment associated with subretinal serous fluid, exudates, and/or blood. Other manifestations of neovascular AMD may include retinal hard exudates, subretinal and sub-RPE fibrovascular proliferation, and/or disciform scar.

Other classification schemes, or modified forms of the AREDS scheme, may be used, such as the ICD10 classification system. The terms “extrafoveal” and “nonsubfoveal” lesions are used interchangeably herein, and refer to lesions having a distance from the atrophy junction to the center of the fovea of greater than zero. The terms “subfoveal” and “foveal” lesions are used interchangeably herein, and refer to lesions having a distance from the atrophy junction to the center of the fovea that is not greater than zero. In some embodiments, such distance is assessed using autofluorescence.

In some embodiments, age-related macular degeneration can be divided into 3 main clinical stages (early, intermediate, and late) based on overall disease severity (Ferris et al., Ophthalmology. 2013; 120:844-851), with decreased quality of life and significant visual impairment occurring during the late stage. Late AMD can be subdivided into the exudative form, which is characterized by the presence of macular neovascularization (MNV), and the nonexudative form, known as geographic atrophy (GA), which is characterized by progressive loss of macular photoreceptors, retinal pigment epithelium (RPE), and choriocapillaris (Holz et al., Ophthalmology. 2014; 121:1079-1091; Barbazetto et al., Arch Ophthalmol. 2003; 121:1253-1268). Geographic atrophy is also referred to as complete RPE and outer retinal atrophy (Holz et al., JAMA Ophthalmol. 2018; 136:666-677). The exudative form of AMD (eAMD) can be treated with intravitreal injections of vascular endothelial growth factor (VEGF) A inhibitors (Finger et al., BMC Ophthalmol. 2020; 20:294). Although anti-VEGF therapy has been shown to improve vision and reduce the risk of severe vision loss over several years in eyes with eAMD (Heier et al., Ophthalmology. 2012; 119:2537-2548; Rosenfeld et al., N Engl J Med. 2006; 355:1419-1431; Gillies et al., Ophthalmology. 2015; 122:1837-1845), the nonexudative form of AMD can continue to progress to macular atrophy, independent of the exudative process (Gune et al., Ophthalmology. 2020; 127:523-532). For most patients with late AMD, macular atrophy is the characteristic end stage of the disease process, resulting in progressive and irreversible loss of visual function as the area of macular atrophy enlarges. See also Wykoff et al., Ophthalmology 128:1325-1336, 2021.

### V. C5 Inhibitors

In some embodiments, a subject treated with a compstatin analog described herein, e.g., pegcetacoplan, has received one or more C5 inhibitors before treatment with the compstatin analog, receives one or more C5 inhibitors in combination with at least one dose of the compstatin analog, and/or continues to receive one or more C5 inhibitors during the entire treatment with the compstatin analog.

C5 inhibitors are known and/or commercially available. Non-limiting examples of C5 inhibitors include, e.g., eculizumab, ALXN1210 (ravulizumab), SKY59 (crovalimab), LFG316, REGN3918, ABP959, RA101495, Coversin, and ALNCC5 (described in, e.g., Risitano et al., Frontiers Immunology 10:1157 (2019)). Additional C5-targeting agents are described in, e.g., U.S. Pat. Nos. 9,718,880 and 9,079,949; and PCT Publs. WO2004106369; WO2010015608; WO2013093762; WO/2014/160129; WO2015134894; WO2015191951; WO/2016/040589; WO/2016/044419; WO2016098356; WO2016117346; WO2016123371; WO/2016/201301; WO2017104779; WO2017105939; WO2017212375; WO2017212391; WO/2017/214518; WO2017/217524; WO2017218515; WO2018106859; WO2018143266; WO2018165062; WO2018183449; WO2019014360; WO2019023564; WO2019084438; WO2019112984; WO2019118556; and WO2020006266.

In some embodiments, the C5 inhibitor is an anti-C5 antibody, e.g., an anti-C5 monoclonal antibody. In some embodiments, a C5 inhibitor is eculizumab or ravulizumab. In some embodiments, a C5 inhibitor is an antibody that binds to the same epitope as eculizumab or ravulizumab. In some embodiments, a C5 inhibitor is an antibody that competes for binding to C5 with eculizumab or ravulizumab. In some embodiments, a C5 inhibitor includes the same or substantially the same amino acid sequence as eculizumab or ravulizumab, or an antigen binding portion thereof.

### VI. Pharmaceutical Compositions and Administration

The disclosure provides and/or utilizes a variety of compositions comprising a compstatin analog. In various embodiments, a composition can have any feature or combination of features discussed herein so long as they are not mutually exclusive. The invention provides embodiments of such compositions, and methods of use thereof, in which the compstatin analog is any compstatin analog, e.g., an analog described herein.

Suitable preparations, e.g., substantially pure preparations of a compstatin analog or other active agent, may be combined with pharmaceutically acceptable carriers or vehicles, etc., to produce an appropriate pharmaceutical composition. The term “pharmaceutically acceptable carrier or vehicle” refers to a non-toxic carrier or vehicle that does not destroy the pharmacological activity of the compound with which it is formulated. One of skill in the art will understand that a carrier or vehicle is “non-toxic” if it is compatible with administration to a subject in an amount appropriate to deliver the compound without causing undue toxicity. Pharmaceutically acceptable carriers or vehicles that may be used in the compositions of this invention include, but are not limited to, water, physiological saline, Ringer's solution, sodium acetate or potassium acetate solution, 5% dextrose, and the like. The composition may include other components as appropriate for the formulation desired, e.g., as discussed herein. Supplementary active compounds, e.g., compounds independently useful for treating a subject suffering from a complement-mediated disorder, can also be incorporated into the compositions. The invention provides such pharmaceutical compositions comprising a compstatin analog and, optionally, a second active agent useful for treating a subject suffering from AMD.

In some embodiments, the invention provides a pharmaceutically acceptable composition suitable for administration to humans, packaged together with a label approved by a government agency responsible for regulating pharmaceutical agents, e.g., the U.S. Food & Drug Administration. In some embodiments, the invention provides a pharmaceutical kit or pack for use in performing an intravitreal injection of a composition, e.g., pegcetacoplan, as described herein. In some embodiments, a pharmaceutical kit comprises one or more sterile needles. In some embodiments, a pharmaceutical kit comprises: (a) a transfer needle (e.g., a filtered transfer needle) and/or (b) a needle for injection. In some embodiments, the invention provides a pharmaceutical kit or pack comprising: (a) a single use vial comprising a pharmaceutical composition comprising pegcetacoplan, as described herein, (b) a transfer needle (e.g., a filtered transfer needle), and/or (c) a needle for injection. In some embodiments, a transfer needle and/or needle for injection are sterile needles. In some embodiments, a transfer needle and/or needle for injection are for administration of a single dose. In some embodiments, a transfer needle is a sterile 5-micron filter needle. In some embodiments, a needle for injection is a ½ inch, 29-gauge thin-wall injection needle. In some embodiments, a needle for injection is a ½ inch, 29-gauge thin-wall injection needle with a Luer-lock hub. In some embodiments, a transfer needle and/or needle for injection are contained in a sealed plastic tray comprising suitably sized compartments to house the transfer needle and/or needle for injection. In some embodiments, a pharmaceutical kit or pack further comprises a syringe for administration of a composition, e.g., pegcetacoplan, as described herein. In some embodiments, a syringe is a sterile 1-ml Luer-lock syringe. In some embodiments, a sterile 1-ml Luer-lock syringe has a 0.1 ml dose mark. In some embodiments instructions for administration are provided.

A pharmaceutical composition can be administered to a subject by any suitable route of administration including, but not limited to, intravitreally, intravenous, intramuscular, subcutaneously, by inhalation, by nasal delivery, intrathecally, intracranially, intraarterially, orally, rectally, transdermally, intradermally, subdermally, etc. It will be understood that “administration” encompasses directly administering a compound or composition to a subject, instructing a third party to administer a compound or composition to a subject, prescribing or suggesting a compound or composition to a subject, and, as appropriate, other means of making a compound or composition available to a subject.

Pharmaceutical compositions suitable for injectable use (e.g., intravitreal administration) typically include sterile aqueous solutions (where water soluble) or dispersions and sterile powders for the extemporaneous preparation of sterile injectable solutions or dispersion. Sterile solutions can be prepared by incorporating the compound in the required amount in an appropriate solvent, optionally with one or a combination of ingredients such as buffers such as acetates, citrates, lactates or phosphates; agents for the adjustment of tonicity; antibacterial agents such as benzyl alcohol or methyl parabens; antioxidants such as ascorbic acid, glutathione, or sodium bisulfite; chelating agents such as ethylenediaminetetraacetic acid; and other suitable ingredients etc., as desired, followed by filter-based sterilization. One of skill in the art will be aware of numerous physiologically acceptable compounds that may be included in a pharmaceutical composition. pH can be adjusted with acids or bases, such as hydrochloric acid or sodium hydroxide, if desired. Preferably solutions for injection are sterile and acceptably free of endotoxin. In some embodiments, solutions for injection are substantially free of preservative (e.g., are preservative free solutions). A composition is “substantially free” of a particular substance or substances if the composition contains no more than 0.1% weight/volume (w/v) of that substance or substances, e.g., no more than 0.01% w/v, e.g., no more than 0.001% w/v, of that substance or substances.

In certain embodiments a composition comprising a compstatin analog, or a composition to which a compstatin analog is to be added, comprises one or more non-reducing sugars or sugar alcohols. In certain embodiments a composition comprising a compstatin analog, or a composition to which a compstatin analog is to be added, comprises one or more reducing sugars, e.g., dextrose. One of ordinary skill in the art appreciates that a reducing sugar is a sugar that is capable of acting as a reducing agent, typically because it has a free aldehyde group or a free ketone group, e.g., when it assumes an open-chain form.

In some embodiments, compositions provided by the present disclosure include a compstatin analog and do not include a reducing sugar, e.g., dextrose, as a component. In some embodiments a composition comprising a compstatin analog, or to which a compstatin analog is to be added, is free or substantially free of one or more reducing sugars, e.g., dextrose. In certain embodiments a composition comprising a compstatin analog, or to which a compstatin analog is to be added, is free or substantially free of reducing sugars.

In some embodiments of any of the aspects described herein, a composition described herein comprises a compstatin analog described herein and water. In some embodiments of any of the aspects described herein, a composition described herein comprises a compstatin analog described herein, water, and one or both of a tonicity adjusting agent and a buffer substance. In some embodiments of any of the aspects described herein, a composition described herein consists essentially of or consists of a compstatin analog described herein and one or more specified components, e.g., a buffer substance and/or a tonicity adjusting agent. In some embodiments, such a composition consists of such substances in water as a pharmaceutically acceptable carrier. In some embodiments of any of the aspects described herein, a composition comprising a compstatin analog described herein has a pH as described herein.

In certain embodiments the composition comprises one or more excipients in an amount such that the composition is approximately isotonic with respect to normal human plasma. In some aspects, a composition is considered “isotonic with respect to normal human plasma” (also referred to simply as “isotonic”) if the concentration of solutes that cannot freely diffuse across a plasma membrane of a normal human cell, e.g., a normal human red blood cell or an epithelial cell, is between 260 mOsm/kg and 320 mOsm/kg, e.g., between 280 mOsm/kg and 300 mOsm/kg, e.g., 285 mOsm/kg and 295 mOsm/kg. In some embodiments a composition comprising a compstatin analog described herein and a pharmaceutically acceptable carrier has a pH of between 6.5 and 7.5, e.g., between 6.8 and 7.2, e.g., 7.0. In some embodiments, a composition comprising a compstatin analog and a pharmaceutically acceptable carrier has a pH of between 6.0 and 6.5, between 5.5 and 6.0, or between 5.0 and 5.5. In certain embodiments a yet lower pH, e.g., between 4.5 and 5.0 may be used. In particular embodiments, for example, a composition has a pH between 4.6 and 5.4, e.g., between 4.8 and 5.2, between 4.9 and 5.1, e.g., 5.0. In some embodiments the composition further comprises one or more pharmaceutically acceptable buffer substances appropriate to maintain the pH within a selected range (e.g., any of the afore-mentioned ranges). Suitable buffer substances are described herein (e.g., acetates, citrates, lactates or phosphates). In some embodiments the composition additionally or alternately comprises a salt, e.g., any of the pharmaceutically acceptable salts described herein.

In certain embodiments the buffer substance comprises sodium acetate. In certain embodiments the buffer substance, e.g., sodium acetate, is present at a concentration between 10 mM and 50 mM, e.g., 10 mM-15 mM, 15 mM-20 mM, 20 mM-25 mM, 25 mM-30 mM, 30 mM-35 mM, 35 mM-40 mM, 40 mM-50 mM. In particular embodiments the buffer substance is present at a concentration of about 15 mM, about 17.5 mM, about 20 mM, about 22.5 mM, about 25 mM, about 27.5 mM, or about 30 mM. In certain embodiments the sodium acetate may be provided by including acetic acid and sodium acetate trihydrate in the solution. In certain particular embodiments, the composition comprises a phosphate.

It will be appreciated that the compstatin analog and/or additional active agent(s) can be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Also, pharmaceutically-acceptable salts can be prepared as alkaline metal or alkaline earth salts, such as sodium, potassium or calcium salts, if appropriate depending on the identity of the active agent.

It will be understood that the pharmaceutically acceptable carriers, compounds, and preparation methods mentioned herein are exemplary and non-limiting. See, e.g., Remington: The Science and Practice of Pharmacy. 21st Edition. Philadelphia, PA. Lippincott Williams & Wilkins, 2005, for additional discussion of pharmaceutically acceptable compounds and methods of preparing pharmaceutical compositions of various types.

In some aspects, described herein are various doses, dosing regimens, compositions, and methods useful for treating patients by IVT injection, e.g., in a manner that is acceptable to physicians and patients in terms of the time and pressure required to deliver a given volume of the composition through a needle of a given inner diameter or gauge number. Gauge number describes the outer diameter of a hollow needle, with a higher gauge number indicating a smaller outer diameter. Generally, needles having a higher gauge number may be preferred by patients and physicians as they may be associated with (or may be perceived to be associated with) less pain and/or tissue damage as compared with needles having a lower gauge number. Inner diameter depends on both outer diameter and wall thickness. For standard needles, the higher the gauge number, the smaller the inner diameter (e.g., a 27 gauge needle has a larger inner diameter than a 29 gauge needle, which in turn has a larger inner diameter than a 30 gauge needle).

In some aspects, the present disclosure teaches particular utility of certain thin-walled needles for administration of a compstatin analog in accordance with the present invention. For example, in some embodiments a thin wall needle is used for intraocular (e.g., intravitreal) injection of a compstatin analog. Thin wall needles have identical outer diameters to standard needles but larger inner diameters for a given gauge. For example, a thin wall needle may have an internal diameter size that is the same as that of a standard needle of a gauge one to two numbers lower (e.g., a 29 gauge thin wall needle may have an internal diameter that is the same as that of a 27 gauge or 28 gauge standard needle but an outer diameter that is the same as that of a standard 29 gauge needle). An increase in internal diameter can result in a considerable increase in fluid flow for a given pressure and/or a considerable reduction in pressure needed to maintain a given flow. Lower pressure means that less injection force is needed to administer a composition of a given viscosity. In general, low injection force facilitates administration and is therefore typically a desirable feature. In some embodiments a thin wall needle has a given internal diameter that is uniform along the length of the needle. In some embodiments a thin wall needle has an internal diameter that varies along the length of the needle. For example, the diameter may be the same as that of a standard 29 gauge needle at one end of the needle and progress to the diameter of a standard 27 gauge needle at the other end. In some embodiments a microtapered needle may be used. In some embodiments a needle with a scalpel-like tip may be used. The length of the needle may vary. In some embodiments a short needle such as a 5 mm or 6 mm needle may be used. In some embodiments a needle having a length between 6 mm and 8 mm, or between 8 mm and 12 mm may be used. Suitable needles and syringes are available commercially, e.g., from Becton Dickinson and Company (BD), Terumo Corp., etc.

In some embodiments a composition having a given viscosity and/or concentration may be administered using a thin wall needle having a gauge that is one or two numbers higher than the gauge size that is preferably used when a standard needle is used to administer a composition of the same viscosity and/or concentration at a selected flow rate and/or with a selected injection force. For example, in some embodiments a composition that is preferably administered using a 25 gauge standard needle in order to attain a desired flow rate and/or injection force can be administered with such a flow rate and/or injection force using a 26 or 27 gauge thin wall needle. In some embodiments a composition that is preferably administered using a 27 gauge standard needle in order to attain a desired flow rate and/or injection force can be administered with such a flow rate and/or injection force using a 28 or 29 gauge thin wall needle. In some embodiments a composition that is preferably administered using a 29 gauge standard needle in order to attain a desired flow rate and/or injection force can be administered with such a flow rate and/or injection force using a 30 or 31 gauge thin wall needle.

In some embodiments a composition is administered using a syringe with one or more design features that reduce friction and/or required injection force, such as a relatively short barrel and/or relatively large plunger size.

In some embodiments, a composition, e.g., pegcetcoplan, is administered using a sterile 1-ml Luer-lock syringe. In some embodiments, a sterile 1-ml Luer-lock syringe has a 0.1 ml dose mark. In some embodiments, such a syringe is provided in a kit comprising a transfer needle (e.g., a filtered transfer needle) and/or needle for injection as described herein.

In some aspects, described herein are various doses, dosing regimens, compositions and methods of use for treating a patient in need of treatment for a complement-mediated eye disorder that affects the posterior segment of the eye, e.g., the retina, by intravitreal (IVT) administration of a compstatin analog. In some embodiments, the eye disorder is AMD, e.g., advanced AMD (geographic atrophy (GA) or neovascular AMD). In some embodiments a composition, e.g., a pharmaceutical composition, comprises a compstatin analog comprising a PEG having a molecular weight of about 40 kD (e.g., pegcetacoplan) at a concentration of about 150 mg/ml.

In some embodiments a dose for intravitreal injection of a compstatin analog (e.g., comprising two compstatin analog moieties and a PEG having a molecular weight of about 40 kD) is 5 mg-20 mg. In some embodiments the dose is about 10 mg. In some embodiments the dose is about 10 mg, about 11 mg, about 12 mg, about 13 mg, about 14 mg, about 15 mg, about 16 mg, about 17 mg, about 18 mg, about 19 mg, or about 20 mg. In certain particular embodiments the dose is about 15 mg. In some embodiments, any of the afore-mentioned doses is administered by intravitreal injection in a volume of between about 90 and about 110 microliters, e.g., in a volume of about 100 microliters.

In certain embodiments a composition, e.g., a composition that may be used for IVT administration, e.g., for treatment of AMD, comprises about 150 mg/ml compstatin analog (e.g., comprising a PEG of about 40 kD, e.g., pegcetacoplan), and one or more excipients selected from the group consisting of NaCl, trehalose, and sorbitol. In certain embodiments any of the compositions comprising about 150 mg/ml compstatin analog has a concentration between 140 mg/ml and 150 mg/ml, e.g., between 145 mg/ml and 155 mg/ml, e.g., between 148 mg/ml and 152 mg/ml, e.g., 150 mg/ml compstatin analog. In certain embodiments any of the compositions comprises about 20 mM sodium acetate. In certain embodiments any of the compositions has a pH between 4.8 and 5.2, e.g., about 5.0.

In some embodiments the composition comprises about 150 mg/ml compstatin analog and between 0.45% and 0.60% w/v NaCl, e.g., between 0.45% and 0.50%, between 0.50% and 0.55%, or between 0.55% and 0.60% NaCl. In certain embodiments the composition comprises between 0.51% NaCl and 0.54% NaCl. In certain embodiments the composition comprises between 0.52% NaCl and 0.53% NaCl. In particular embodiments the composition comprises 0.500% NaCl. In particular embodiments the composition comprises 0.505% NaCl. 0.510% NaCl. In particular embodiments the composition comprises 0.515% NaCl. In particular embodiments the composition comprises 0.520% NaCl. In particular embodiments the composition comprises 0.525% NaCl. In particular embodiments the composition comprises s 0.530% NaCl. In particular embodiments the composition comprises 0.535% NaCl. In particular embodiments the composition contains 0.540% NaCl. In particular embodiments the composition comprises 0.545% NaCl. In particular embodiments the composition comprises 0.550% NaCl.

In certain embodiments the composition comprises about 150 mg/ml compstatin analog and between 2.5% and 4.5% w/v sorbitol w/v, e.g., between 2.5% and 3.0%, between 3.0% and 3.5%, between 3.5% and 4.0%, or between 4.0% and 4.5% sorbitol. In particular embodiments the composition comprises 3.0% sorbitol. In particular embodiments the composition comprises 3.1% sorbitol. In particular embodiments the composition comprises 3.2% sorbitol. In particular embodiments the composition comprises 3.3% sorbitol. In particular embodiments the composition comprises 3.4% sorbitol. In particular embodiments the composition comprises 3.5% sorbitol.

In certain embodiments the composition comprises about 150 mg/ml compstatin analog and between 6.0% and 8.0% w/v trehalose, e.g., between 6.0% and 6.5%, between 6.5% and 7.0%, between 7.0% and 7.5%, or between 7.5% and 8.0% trehalose. In particular embodiments the composition comprises 6.5% trehalose. In particular embodiments the composition comprises 6.6% trehalose. In particular embodiments the composition comprises 6.7% trehalose. In particular embodiments the composition comprises 6.8% trehalose. In particular embodiments the composition comprises 6.9% trehalose. In particular embodiments the composition comprises 7.0% trehalose. In some embodiments, the composition comprises about 5.0% to about 6.0% trehalose, about 5.0% to about 5.5% trehalose, about 5.5% to about 6.0% trehalose, about 5.0% to about 5.3% trehalose, or about 5.2% to about 5.5% trehalose. In particular embodiments, the composition comprises about 5.3% to about 5.4% (e.g., about 5.38%) trehalose. In certain embodiments, the trehalose may be provided by including trehalose dehydrate in the solution.

In some embodiments, the osmolality of a solution comprising a compstatin analog may be measured at a dilution such that the concentration of compstatin analog is about 75 mg/ml or less. A solution comprising a compstatin analog and, optionally, an osmolality modifier, may be diluted with water by an appropriate dilution factor such that the concentration of the compstatin analog is 75 mg/ml or less, e.g., between 25 mg/ml and 40 mg/ml or between 40 mg/ml and 75 mg/ml. The osmolality of the resulting solution is measured and then multiplied by the dilution factor to correct for the dilution. For example, a solution having a compstatin analog concentration of 100 mg/ml may be diluted by a factor of 2, i.e., to a concentration of 50 mg/ml. The osmolality of the resulting solution is measured and multiplied by 2 to yield the osmolality of the original 100 mg/ml solution. As another example, a solution having a compstatin analog concentration of 150 mg/ml may be diluted by a factor of 2, i.e., to a concentration of 75 mg/ml. The osmolality of the resulting solution is measured and multiplied by 2 to yield the osmolality of the original 100 mg/ml solution.

In some embodiments a composition comprising a compstatin analog may have a selected osmolality. In some embodiments a composition comprising a compstatin analog, e.g., a composition for ocular, e.g., intravitreal, administration, has an osmolality of between 150 milliosmoles/kilogram (mOsm/kg) and 600 mOsm/kg, e.g., between 180 mOsm/kg and 500 mOsm/kg, e.g., between 200 mOsm/kg and 400 mOsm/kg. In certain embodiments a composition comprising a compstatin analog has an osmolality between 250 mOsm/kg and 380 mOsm/kg, e.g., between 275 mOsm/kg and 350 mOsm/kg. In certain embodiments a composition comprising a compstatin analog has an osmolality of between 275 mOsm/kg and 285 mOsm/kg, between 285 mOsm/kg and 295 mOsm/kg, between 295 mOsm/kg and 305 mOsm/kg, or between 305 mOsm/kg and 315 mOsm/kg. In particular embodiments the osmolality of the composition is 300 mOsm/kg. In some embodiments the osmolality of a composition may be measured using a vapor pressure depression osmometer. In some embodiments the osmolality of a composition may be measured using a membrane osmometer. In some embodiments the osmolality of a composition may be measured using a freezing point depression osmometer.

In some embodiments a dose of a composition comprising a compstatin analog is administered by ocular administration (e.g., IVT injection) once a month (e.g., 30 days), every 6 weeks, or every 2 months (i.e., every other month, (e.g., 60 days)). In some embodiments a dose of a composition comprising a compstatin analog is administered by IVT injection every 3 months, every 4 months, every 5 months, or every 6 months, or less frequently, e.g., every 9 months, every year). Thus in some embodiments a patient may receive between 1 and 6 injections per year, typically at approximately equal intervals. In some embodiments, a patient may receive between 6 and 12 injections per year, typically at approximately equal intervals. In some embodiments a patient is initially treated with monthly injections (e.g., for the first 3-6 months or the first 6-12 months), followed by less frequent administration (e.g., every 2, 3, 4, 5, or 6 months, or less frequently, e.g., every 9 months, every year).

In some embodiments, a pharmaceutical composition described herein includes pegcetacoplan at 15 mg/0.1 mL (150 mg/mL), as a sterile, aqueous, acetate-buffered trehalose solution for intravitreal administration. In some embodiments, such pharmaceutical composition is included in a single-use 2R vial containing 60 mg of pegcetacoplan in 0.4 mL solution, 75 mg of pegcetacoplan in 0.5 mL solution, 150 mg of pegcetacoplan in 1.0 mL solution, 45 mg of pegcetacoplan in 0.3 mL solution, or 30 mg of pegcetacoplan in 0.2 mL solution. In some embodiments, each vial is designed to deliver a single dose of 0.1 mL solution containing 15 mg of pegcetacoplan. Those skilled in the art, reading the present disclosure, will appreciate that, in accordance with standard practice in the field, a container containing a particular volume, as described herein my include an additional volume sufficient to permit the designated particular volume (e.g., unit dose) to be withdrawn from the container for administration.

In some embodiments, a pharmaceutical composition includes 15 mg/0.1 mL pegcetacoplan, 16-22 mM acetate buffer (e.g., about 16 mM, about 17 mM, about 18 mM, about 19 mM, about 20 mM, about 21 mM, or about 22 mM acetate buffer), 5% to 6% trehalose (e.g., about 5%, about 5.1%, about 5.2%, about 5.3%, about 5.4%, about 5.5%, about 5.6%, about 5.7%, about 5.8%, about 5.9%, or about 6.0% trehalose, e.g., about 5.38% trehalose), with a pH of about 4.8 to about 5.2 (e.g., about 5.0).

In some embodiments, a pharmaceutical composition includes the following, in a 0.1 mL volume: 15 mg Pegcetacoplan, about 5.5 mg to about 6.5 mg trehalose dihydrate (e.g., about 5.5 mg, about 5.6 mg, about 5.7 mg, about 5.8 mg, about 5.9 mg, about 6 mg, about 6.1 mg, about 6.2 mg, about 6.3 mg, about 6.4 mg, or about 6.5 mg trehalose dihydrate (e.g., about 5.95 mg trehalose dihydrate)), about 0.085 mg to about 0.095 mg glacial acetic acid (e.g., about 0.085 mg, about 0.0855 mg, about 0.086 mg, about 0.0865 mg, about 0.087 mg, about 0.0875 mg, about 0.088 mg, about 0.0885 mg, about 0.089 mg, about 0.0895 mg, about 0.09 mg, about 0.0905 mg, about 0.091 mg, about 0.0915 mg, about 0.092 mg, about 0.0925 mg, about 0.093 mg, about 0.0935 mg, about 0.094 mg, about 0.0945 mg, or about 0.095 mg glacial acetic acid (e.g., about 0.0895 mg glacial acetic acid)), about 0.03 mg to about 0.04 mg sodium acetate trihydrate (e.g., about 0.031 mg, about 0.032 mg, about 0.033 mg, about 0.034 mg, about 0.035 mg, about 0.036 mg, about 0.037 mg, about 0.038 mg, about 0.039 mg, or about 0.04 mg sodium acetate trihydrate (e.g., about 0.0353 mg sodium acetate trihydrate)), water for injection, and optionally sodium hydroxide and/or additional glacial acetic acid in an amount sufficient to adjust the pH of the pharmaceutical composition to about 4.8 to about 5.2 (e.g., about 5.0).

### VII. Treatment Methods

In some embodiments, a compstatin analog described herein, e.g., pegcetacoplan, is used to treat geographic atrophy (GA). In some embodiments, a compstatin analog described herein, e.g., pegcetacoplan, is administered to an eye of a subject having or suffering from GA. In some embodiments, one eye of the subject has GA, and pegcetacoplan is administered to that eye. In some embodiments, both eyes of the subject have GA, and pegcetacoplan is administered to both eyes. In some embodiments, one eye of the subject has GA and one eye has wet AMD, and pegcetacoplan is administered to the eye having GA. In some embodiments, one eye of the subject has GA, and a second eye of the subject has wet AMD but not GA, and pegcetacoplan is administered to the first eye. In some embodiments, one eye of the subject has GA, and a second eye of the subject has wet AMD and has GA, and pegcetacoplan is administered to the first eye. In some embodiments, one eye of the subject has GA, and a second eye of the subject has wet AMD and has GA, and pegcetacoplan is administered to the second eye. In some embodiments, one eye of the subject has GA, and a second eye of the subject has wet AMD and has GA, and pegcetacoplan is administered to the first eye and to the second eye.

In some embodiments, a compstatin analog described herein, e.g., pegcetacoplan, is administered, e.g., intravitreally, to the eye of the subject as a pharmaceutical composition that includes pegcetacoplan at 15 mg/0.1 mL (150 mg/mL). In some embodiments, the composition is administered to the eye of the subject once a month (e.g., 30 days), e.g., for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer. In some embodiments, the composition is administered to the eye of the subject every other month (e.g., 60 days), e.g., for about 8 weeks, about 16 weeks, about 24 weeks, about 32 weeks, about 40 weeks, about 48 weeks, about 1.2 years, 1.4 years, 1.6 years, 1.8 years, 2 years, 3 years, 4 years, 5 years, or longer. In some embodiments, a specific improvement (e.g., a statistically significant or clinically significant improvement) of one or more GA symptoms or parameters is achieved in the subject, e.g., one or more target levels described herein is achieved.

In some embodiments, a subject may be monitored and/or evaluated for elevated intraocular pressure (IOP) prior to administration, e.g., intravitreal injection, of a composition described herein. In some embodiments, IOP is measured using tonometry. In some embodiments, a subject may be administered ocular hypotensive medication to lower IOP. In some embodiments, prior to administration, e.g., intravitreal injection of a composition described herein, IOP is about ≤21 mmHg. In some embodiments, an eye of the subject may be softened and/or decompressed prior to administration, e.g. intravitreal injection, of a composition described herein. In some embodiments, an eye of the subject may be softened and/or decompressed using a cotton-tipped applicator. In some embodiments, a cotton-tipped applicator may be saturated with sterile topical anesthetic drops. In some embodiments, an eye of the subject may be softened and/or decompressed by pushing against the globe with the cotton-tipped applicator at the planned injection site for between about 30 to about 60 seconds. In some embodiments, softening and/or decompressing an eye of a subject prior to administration, e.g., intravitreal injection, of a composition described herein may prevent and/or minimize an increase in IOP.

In some embodiments, methods of the disclosure include diagnosing the subject as suffering from or at risk of geographic atrophy (GA), e.g., GA secondary to age related macular degeneration (AMD), and/or selecting the subject for treatment with pegcetacoplan. In some embodiments, diagnosing may comprise performing fundus autofluorescence (FAF) and/or optical coherence tomography (OCT). In some embodiments, the subject is determined to have foveal GA (e.g., in one or both eyes) and is selected for treatment. In some embodiments, the subject is determined to have extrafoveal GA (e.g., solely extrafoveal GA) (e.g., in one or both eyes) and is selected for treatment. In some embodiments, the subject is selected for treatment if total GA area is ≥2.5 mm² (e.g., ≥2.5 mm² and ≤17.5 mm²).

In some embodiments an eye treated with pegcetacoplan by monthly IVT injection has a risk of about 6% or less per year for developing exudation. In some embodiments an eye treated with pegcetacoplan by every other month IVT injection has a risk of less than about 4% per year for developing exudation. In some embodiments the risk of developing exudation for an eye treated with pegcetacoplan by monthly IVT injection is about 2.5 (or less) times that of a control eye not treated with pegcetacoplan, e.g., between about 2.0 and about 2.5 times that of a control eye not treated with pegcetacoplan, e.g., over a specified time period such as a year. In some embodiments the risk of developing exudation for an eye treated with pegcetacoplan by monthly IVT injection is about 1.7 (or less) times that of a control eye not treated with pegcetacoplan, e.g., between about 1.5 and about 1.7 times that of a control eye not treated with pegcetacoplan, e.g., over a specified time period such as a year.

Methods described herein can include preparing and/or providing a report, such as in electronic, web-based, or paper form. The report can include one or more outputs from a method described herein, e.g., a subject's response to a treatment described herein. In some embodiments, a report is generated, such as in paper or electronic form, which identifies one or more endpoints described herein for a subject, and optionally, a recommended course of therapy. In some embodiments, the report includes an identifier for the subject. In some embodiments, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a subject, e.g., identified in the report. For example, the report can include information, or a recommendation, on the administration of a compstatin analog described herein, e.g., pegcetacoplan, to the subject. The report can be delivered, e.g., to an entity described herein, within 7, 14, 21, 30, or 45 days from performing a method described herein.

In some embodiments, a report is generated to memorialize each time a subject is assessed using a method described herein. The subject can be reevaluated at intervals, such as every month, every two months, every six months or every year, or more or less frequently, to monitor the subject for responsiveness to compstatin analog, e.g., pegcetacoplan, and/or for an improvement in one or more GA symptoms, e.g., described herein. In some embodiments, the report can record at least the treatment history of the subject.

In some embodiments, the method further includes providing a report to another party. The other party can be, for example, the subject, a caregiver, a physician, an oncologist, a hospital, clinic, third-party payor, insurance company or a government office.

The disclosure encompasses administration of a compstatin analog in combination with additional therapy. Such additional therapy may include administration of any agent(s) used in the art or potentially useful for treating a subject suffering from the disease. For example, in some embodiments a compstatin analog is administered in combination with a C5 inhibitor (e.g., eculizumab or any of the other C5 inhibitors mentioned herein or known in the art) to a patient, e.g., a patient with AMD. In some embodiments a compstatin analog is administered in combination with an anti-vascular endothelial growth factor (VEGF) agent to a subject, e.g., a subject with wet AMD. Anti-VEGF agents include antibodies that bind to VEGF such as ranibizumab (Lucentis). bevacizumab (Avastin), brolucizumab (Novartis), faricimab (Roche), and polypeptides comprising a soluble portion of VEGF receptor such as aflibercept (Eylea, also known as VEGF-Trap) and conbercept (Chengdu Kanghong Biotech). In particular embodiments the anti-VEGF agent is ranibizumab or aflibercept.

In some embodiments a subject treated with a compstatin analog, e.g., pegcetacoplan, may develop exudation in the treated eye. In some embodiments a subject who exhibits evidence of exudation in an eye being treated with pegcetacoplan may be started on therapy with an anti-VEGF agent administered to said eye. In some embodiments a method of treatment comprises administering pegcetacoplan to an eye suffering from GA and evaluating said eye for development of exudation (e.g., related to active CNV). Evaluating may comprise performing fundus examination and/or optical coherence tomography (OCT). In some embodiments, a subject may be determined to have exudation based on detection of, e.g., subretinal fluid, intraretinal fluid, cystoid macular edema, and/or serous pigment epithelial detachment. In some embodiments evaluating for presence of exudation may comprise performing fluorescein angiography (FA) and/or optical coherence tomography angiography (OCT-A). In some embodiments a subject may be confirmed to have exudation by FA and/or OCT-A. In some embodiments an evaluation procedure may be performed when the subject is visiting a health care provider, e.g., an eye care professional, for administration of pegcetacoplan. In some embodiments, a subject may be instructed to report to a health care provider, e.g., an eye care professional, any symptoms of exudation. In some embodiments a subject determined to exhibit exudation (e.g., onset of exudation) in an eye treated with pegcetacoplan may be started on treatment of said eye with an anti-VEGF agent. In some embodiments a method of treatment comprises determining that an eye being treated with pegcetacoplan exhibits evidence of exudation and administering an anti-VEGF agent to said eye. In some embodiments a method of treatment comprises determining that an eye having wet AMD exhibits GA and administering pegcetacoplan and an anti-VEGF agent to said eye. In some embodiments a method of treatment comprises determining that an eye has wet AMD and GA and administering pegcetacoplan and an anti-VEGF agent to said eye. In some embodiments an anti-VEGF agent may be an agent that is approved for treatment of neovascular AMD and is administered according to its approved dosing regimen for treatment of neovascular AMD as described in the prescribing information for such anti-VEGF agent. In some embodiments an eye may be treated by IVT injection with pegcetacoplan and an anti-VEGF agent on the same day. In some embodiments an eye may be treated by IVT injection with pegcetacoplan and an anti-VEGF agent on different days. In embodiments wherein an eye is treated with pegcetacoplan and an anti-VEGF agent on the same day, the anti-VEGF agent may be administered before administration of pegcetacoplan. In some embodiments administration of the anti-VEGF agent and pegcetacoplan are administered at least 30 minutes apart on the same day. In some embodiments, administration of the anti-VEGF agent is administered at least 30 minutes before administration of pegcetacoplan. In some embodiments, wherein an eye is treated with pegcetacoplan and an anti-VEGF agent on the same day, the anti-VEGF agent may be administered after administration of pegcetacoplan. In some embodiments, administration of the anti-VEGF agent and pegcetacoplan are administered at least 30 minutes apart on the same day. In some embodiments, administration of the anti-VEGF agent is administered at least 30 minutes after administration of pegcetacoplan.

When two or more therapies (e.g., compounds or compositions) are used or administered “in combination” with each other, they may be given at the same time, within overlapping time periods, or sequentially (e.g., separated by up to 2 weeks in time, or more, e.g., separated by up to about 4, 6, 8, or 12 weeks in time), in various embodiments of the invention. They may be administered via the same route or different routes. In some embodiments, the compounds or compositions are administered within 48 hours of each other. In some embodiments, a compstatin analog can be given prior to or after administration of the additional compound(s), e.g., sufficiently close in time that the compstatin analog and additional compound(s) are present at useful levels within the body at least once. In some embodiments, the compounds or compositions are administered sufficiently close together in time such that no more than 90% of the earlier administered composition has been metabolized to inactive metabolites or eliminated, e.g., excreted, from the body, at the time the second compound or composition is administered.

In some embodiments, a composition that includes both a compstatin analog and additional compound(s) is administered.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. The example is provided for illustrative purposes only. It is not to be construed as limiting the scope or content of the disclosure in any way.

### EXEMPLIFICATION

### Example 1-Phase III, Multi-Center, Randomized, Double-Masked, Sham-Controlled Studies to Compare the Efficacy and Safety of Intravitreal APL-2 Therapy with Sham Injections in Patients with Geographic Atrophy (GA) Secondary to Age-Related Macular Degeneration (AMD)

This Example describes 12-month results from two Phase 3 multi-center, randomized, double-masked, sham-controlled studies (“DERBY” and “OAKS”) to compare the efficacy and safety of intravitreal APL-2 (pegcetacoplan, also referred to as “Study Drug” in this Example) therapy with sham injections in patients with geographic atrophy (GA) secondary to age-related macular degeneration (AMD).

### Methods

### Patient Selection

To participate in the studies, subjects must have been diagnosed with GA of the macula secondary to AMD in the study eye. At screening, male and female subjects were required to fulfill all of the following inclusion criteria to be eligible for participation in the study. If both eyes met the inclusion criteria, the eye with the worst normal luminance visual acuity at the screening visit was designated as the study eye. If both eyes had the same visual acuity, the right eye was selected as the study eye. Ocular-specific inclusion criteria applied to the study eye only, unless otherwise specified.

### Inclusion Criteria

1. Age ≥60 years.
2. Normal Luminance best corrected visual acuity of 24 letters or better using Early Treatment Diabetic Retinopathy Study (ETDRS) charts (approximately 20/320 Snellen equivalent).
3. Clinical diagnosis of GA of the macula secondary to AMD as determined by the investigator and confirmed by the reading center.
4. The GA lesion must have met the following criteria as determined by the central reading center's assessment of FAF imaging at screening:
   a. Total GA area must be >2.5 and ≤17.5 mm² (1 and 7 disk areas [DA] respectively).
   b. If GA was multifocal, at least 1 focal lesion must be ≥1.25 mm² (0.5 DA), with the overall aggregate area of GA as specified above in 4a.
   c. The entire GA lesion must be completely visualized on the macula centered image and must be able to be imaged in its entirety and not contiguous with any areas of peripapillary atrophy.
   d. Presence of any pattern of hyperautofluorescence in the junctional zone of GA. Absence of hyperautofluorescence (ie, pattern=none) was exclusionary.
5. Adequate clarity of ocular media, adequate pupillary dilation, and fixation to permitted the collection of good quality images as determined by the investigator.
6. Met the following criteria related to microperimetry:
   a. Able to detect fixation target.
   b. Total elapsed time to complete the 10-2 68-point exam was ≤30 minutes in duration.
   c. Fixation losses must be ≤20%.
   d. Subject was willing and able to undertake microperimetry assessment in the opinion of the investigator.
7. Female subjects must have been:
   a. Women of non-childbearing potential (WONCBP), or
   b. Women of childbearing potential (WOCBP) with a negative serum pregnancy test at screening and agreed to use protocol defined methods of contraception for the duration of the study and refrain from breastfeeding for the duration of the study.
8. Males with female partners of childbearing potential must agree to use protocol defined methods of contraception and agree to refrain from donating sperm for the duration of the study.
9. Willing and able to give informed consent and to comply with the study procedures and assessments.

Subjects were excluded from the studies based on the following exclusion criteria. Ocular specific exclusion criteria applied to the study eye only, unless otherwise specified.

### Exclusion Criteria

1. GA secondary to a condition other than AMD such as Stargardt disease, cone rod dystrophy, or toxic maculopathies like plaquenil maculopathy in either eye.
2. Spherical equivalent of the refractive error demonstrating >6 diopters of myopia or an axial length >26 mm.
3. Any history or active CNV in the study eye, associated with AMD or any other cause, including any evidence of retinal pigment epithelium rips or evidence of neovascularization anywhere based on SD-OCT imaging and/or fluorescein angiography as assessed by the reading center.
4. Presence of an active ocular disease that in the opinion of the investigator compromised or confounded visual function, including but not limited to, uveitis, other macular diseases (eg, clinically significant epiretinal membrane [ERM], full thickness macular hole) or uncontrolled glaucoma/ocular hypertension). Benign conditions in the opinion of the investigator such as peripheral retina dystrophy were not exclusionary.
5. Intraocular surgery (including lens replacement surgery) within 3 months prior to randomization.
6. History of laser therapy in the macular region.
7. Aphakia or absence of the posterior capsule. Note: YAG laser posterior capsulotomy for posterior capsule opacification done at least 60 days prior to screening was not exclusionary.
8. Any ocular condition other than GA secondary to AMD that may have required surgery or medical intervention during the study period or, in the opinion of the investigator, could have compromised visual function during the study period.
9. Any contraindication to IVT injection including current ocular or periocular infection.
10. History of prior intravitreal injection.
11. Unable to perform microperimetry reliably in the opinion of the investigator.
12. Prior participation in another interventional clinical study for intravitreal therapies in either eye (including subjects receiving sham).
13. Prior participation in another interventional clinical study for geographic atrophy in either eye including investigational oral medication and placebo.
14. Participation in any systemic experimental treatment or any other systemic investigational new drug including within 6 weeks or 5 half-lives of the active ingredient (whichever was longer) prior to the start of study treatment. Note: clinical trials solely involving observation, over-the-counter vitamins, supplements, or diets were not exclusionary.
15. Medical or psychiatric conditions that, in the opinion of the investigator, would make consistent follow-up over the 24-month treatment period unlikely, or would make the subject an unsafe study candidate.
16. Any screening laboratory value (hematology, serum chemistry or urinalysis) that in the opinion of the investigator was clinically significant and not suitable for study participation.
17. Known hypersensitivity to fluorescein sodium for injection or hypersensitivity to pegcetacoplan or any of the excipients in pegcetacoplan solution.

### Study Design

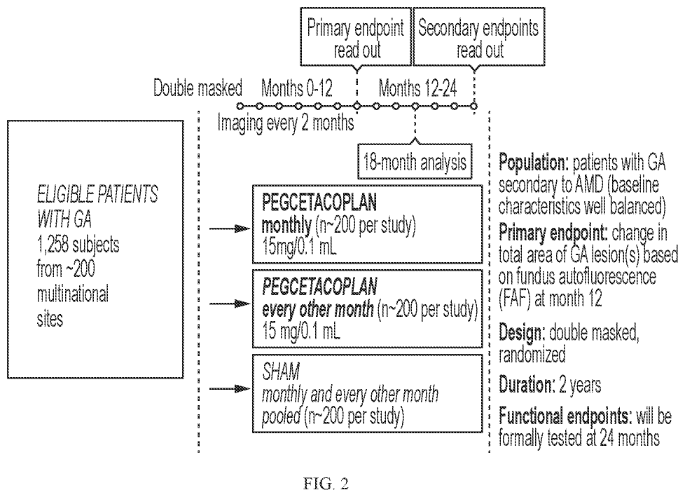

The design of the DERBY and OAKS studies are schematically depicted in FIG. 2. Each subject was assigned a unique screening number after signing the informed consent. Subjects who completed the study screening assessments and met all the eligibility criteria entered the study and were randomized on Day 1. As part of the screening process, the reading center evaluated FAF, OCT, Digital Color Fundus Photograph (DCFP), Near Infrared Reflectance (NIR), and fluorescein angiography (FA) to provide an objective assessment of subject eligibility. Subjects were randomized 2:2:1:1 to receive treatment with Study Drug monthly (PM), Study Drug every other month (PEOM), sham injection monthly (SM) or sham injection every other month (SEOM), respectively.

All randomized subjects returned every month to the clinical site for assessments and additional pegcetacoplan or sham injections according to their randomization scheme until Month 12. From Month 12 onwards, subjects return to the clinical site based on their randomized treatment schedule (monthly or EOM) and follow the treatment regimen and assessments outlined in the Visit Schedule until Month 24. Safety was assessed throughout the study by a number of evaluations including: monitoring of AEs, preinjection and postinjection monitoring, blood and urine samples collected, physical examination, vital signs, and follow-up phone calls. Blood samples were also be collected for anti-therapeutic antibodies, genotyping, and clinical repository (if the subject consented to this portion).

### Study Treatments

The Study Drug was pegcetacoplan (also referred to as “APL-2”) (see FIG. 1), which was provided for intravitreal injection at 15 mg/0.1 mL (150 mg/mL) as a sterile, isotonic solution of pegcetacoplan in acetate-buffer, pH 5.0, containing trehalose, supplied in stoppered glass vials.

Screening Period—within 28 Days Prior to Randomization/Treatment (Day −28 to −1)

Visit 1—All Subjects

Note: All ophthalmic procedures (including imaging) were performed on both eyes, except where specified. Demographic information, significant medical/surgical history within the previous 5 years, invasive ocular procedures within the previous 5 years, and concomitant medications used within 30 days prior to screening were collected (including vitamins and all over-the-counter as well as prescription medications). Complete smoking/tobacco history was also collected.

Subject eligibility was then be determined by reviewing the inclusion/exclusion criteria and the study eye was selected. Prior to the administration of fluorescein, blood and urine was collected for safety labs (including blood for human chorionic gonadotropin/follicle-stimulating hormone/luteinizing hormone [HCG/FSH/LH], if applicable) and vital signs along with a physical examination including weight and height was performed.

A complete ophthalmic exam including slitlamp exam of the cornea, iris, anterior chamber, aqueous reaction (cells and flare), dilated fundus exam of the vitreous and retina, and intraocular pressure (IOP) measurement. Normal luminance BCVA was performed prior to dilating the eyes. Images were captured as outlined in the Visit Schedule (FAF, NIR, DCFP, FFA) and forwarded to the reading center for determination of eligibility if applicable. Mesopic microperimetry was performed on both eyes post dilation of the eyes and forwarded to the reading center.

Randomization/Initial Treatment—Day 1—within 28 Days of Screening

Visit 2—All Groups

At this visit, all inclusion/exclusion criteria was reviewed prior to randomization and dosing, including the determination of eligibility by the reading center. Subjects were randomized using the Interactive Web Response (IWR) System. A complete ophthalmic exam including slitlamp exam of the cornea, iris, anterior chamber, lens, and aqueous reaction (cells and flare), dilated fundus exam of the vitreous and retina, and IOP measurement were performed and imaging collected as per the Visit Schedule.

All assessments were performed on the same day. All study visits were scheduled and projected based on the Day 1 visit date.

Blood was drawn for anti-pegcetacoplan antibodies.

Prior to dilating the eyes, all functional tests were performed (NL-BCVA, LL-BCVA, and MNREAD or Radner Reading Charts [in select sites]). Subjects at select sites were trained on how to use the home-based digital applications for visual function and reading speed if the subject decided to participate in this portion. This training occurred after completion of all functional tests (NL-BCVA, LL-BCVA). Tests completed using the digital application at select sites (optional) were completed prior to dilating the eyes but after completion of all functional tests and quality of life measures (NL-BCVA, LL-BCVA, MNREAD or Radner Reading Charts [in select sites], NEI VFQ-25, and FRI). The subject was instructed to take the electronic device home and to complete the digital application weekly on the same day each week, if possible. The quality of life measures (NEI VFQ-25 and FRI) was administered by the masked site staff.

Imaging performed included FAF, SD-OCT, OCT-A (select sites), endothelial cell count (select sites) and NIR and sent to the reading center for evaluation.

Study drug or sham injection was performed by the unmasked physician as described herein and the study eye was monitored post injection. Subjects received a single dose of 15 mg pegcetacoplan/0.1 mL or sham injection intravitreally. A follow-up phone call was scheduled with the subject 4±2 days after randomization Day 1 to assess for any AEs.

### Treatment Phase—24 Months

Months 1-12

### Visits 3-14 (Monthly and Every-Other Month Group)

During this phase, there were clinic visits every month. Dosing and assessments occurred monthly in the monthly pegcetacoplan and sham injection treatment arms. Dosing occurred every other month in the EOM pegcetacoplan and sham injection treatment arms, however, the subjects returned monthly for assessments (with no dose given).

A complete ophthalmic exam including slitlamp exam of the cornea, iris, anterior chamber, lens, and aqueous reaction (cells and flare), dilated fundus exam of the vitreous and retina, and IOP measurement were performed and imaging collected as per the Visit Schedule.

Blood was drawn for safety labs, anti-pegcetacoplan antibodies, and genotyping (Month 2 only) prior to the administration of fluorescein as per the Visit Schedule. Samples were collected for the clinical repository for those subjects that consent to this portion.

Prior to dilating the eyes, all functional tests were performed (NL-BCVA, LL-BCVA, and MNREAD or Radner Reading Charts [in select sites]). Tests completed using the digital application at select sites (optional) were completed prior to dilating the eyes but after completion of all functional tests and quality of life measures (NL-BCVA, LL-BCVA, MNREAD or Radner Reading Charts [in select sites], NEI VFQ-25, and FRI). At select sites the subject was instructed to take the electronic device home and to complete the digital application weekly on the same day each week, if possible. The subject was instructed to bring back the electronic device for the visits specified in the schedule of events. The quality of life measures (NEI VFQ-25 and FRI) was administered by the masked site staff.

Mesopic microperimetry was performed on both eyes (where specified) post dilation of the eyes and forwarded to the reading center. Imaging was performed including FAF, FFA, SD-OCT, OCT-A (select sites), endothelial cell count (select sites) and NIR and sent to the reading center for evaluation per the visit schedule.

### Months 13-24

### Visits 15-26 (Monthly Group) and 15-20 (Every-Other-Month Group)

During this phase, clinic visits follow treatment designation (ie, the monthly subjects return monthly for dosing and assessments and the EOM group returned EOM for dosing and assessments). At select sites, subjects complete the home-based digital assessments weekly during this period.

A complete ophthalmic exam including slitlamp exam of the cornea, iris, anterior chamber, lens, and aqueous reaction (cells and flare), dilated fundus exam of the vitreous and retina, and IOP measurement is performed and imaging is collected as per the Visit Schedule. Blood is drawn for safety labs, genotyping, and anti-pegcetacoplan antibodies prior to the administration of fluorescein as per the Visit Schedule. Samples are collected for the clinical repository for those subjects that consent to this portion.

Prior to dilating the eyes, all functional tests are performed (NL-BCVA, LL-BCVA, and MNREAD or Radner Reading Charts [in select sites]). The quality of life measures (NEI VFQ-25 and FRI) is administered by the masked site staff.

Mesopic microperimetry is performed on both eyes (where specified) post dilation of the eyes and forwarded to the reading center. Imaging is performed including FAF, FFA, SD-OCT, OCT-A (select sites), endothelial cell count (select sites) and NIR and sent to the reading center for evaluation per the visit schedule.

The completion of the 24-month study period occurs approximately 30 days (monthly treatment group) and approximately 60 days (EOM treatment group) after the last visit at which Study Drug is administered. The period following last dose of Study Drug is sufficient to evaluate the safety of pegcetacoplan. At the end of the 24-month study period, subjects have the option to enroll in a separate open-label study.

### Measures of Clinical Efficacy

The primary endpoint in the studies was the change from baseline to Month 12 in total area of GA lesion(s) in the study eye (in mm²) based on Fundus Autofluorescence (FAF). Key secondary endpoints included change from baseline in the mean threshold sensitivity of all points (study eye) assessed by mesopic microperimetry at Month 24; change from baseline in monocular maximum reading speed (study eye), at Month 24 as assessed by MNRead or Radner reading charts (in select sites); change from baseline in mean FRI Index score (subject-level assessment) at Month 24; and change from baseline in NL-BCVA score (study eye) at Month 24 as assessed by Early Treatment Diabetic Retinopathy Study (ETDRS) chart. Additional secondary endpoints included change from baseline in LL-BCVA score (study eye) over time as assessed by ETDRS chart; change from baseline in the total area of GA lesion(s) in the study eye (in mm²) as assessed by FAF over time other than Month 12; change from baseline in monocular critical print size (study eye), as assessed by MNRead or Radner reading charts (in select sites) over time; change from baseline in the NEI VFQ-25 distance activity subscale score (in select sites) (subject level assessment) over time; change from baseline in the number of scotomatous points assessed by mesopic microperimetry over time; and change from baseline in the mean threshold sensitivity within 500 microns outside the GA lesion, (perilesional points) assessed by mesopic microperimetry over time.

Safety endpoints included incidence and severity of ocular and systemic treatment-emergent adverse events; incidence of ADA directed against pegcetacoplan peptide or PEG; incidence of new active CNV in the study eye; incidence of new onset of subclinical CNV in the study eye; incidence of subjects who lost letters based on NL-BCVA categories (≥15, ≥15-<30, ≥30 ETDRS letters); change/shift from baseline in clinical labs and incidence of abnormal lab values; change from baseline in vital signs and incidence of abnormal vital sign results; shift from baseline in ocular examination assessments including slit-lamp examination and indirect ophthalmoscopy; change from baseline in Intra Ocular Pressure (IOP) and incidence of IOP above specified thresholds; and change/shift from baseline in ocular imaging assessments (including specular microscopy; select sites).

### Statistical Analysis

Two sham treatment arms (SM and SEOM) were pooled into a single control (Sham) group for analyses.

The Intent-to-treat (ITT) set consisted of all randomized subjects. Subjects were analyzed in the treatment arm assigned at randomization.

The modified ITT (mITT) set consisted of all randomized subjects who received at least one injection of pegcetacoplan or sham and had baseline and at least one post-baseline value of GA lesion area in the study eye as assessed by FAF. Subjects were analyzed in the treatment arm assigned at randomization.

The Safety set consisted of all subjects randomized who received at least one injection of pegcetacoplan or sham. Subjects were analyzed according to the actual treatment received. In the case a subject received an incorrect injection of study medication than what they were randomized to, subjects were presented under the corresponding pegcetacoplan arm if they received at least one injection of pegcetacoplan during the study and were only be presented under the corresponding sham arm if they did not receive any injections of pegcetacoplan. This population was used for all safety analyses.

The Per-Protocol (PP) sets will be identified separately for Month 12 and Month 24 analysis, respectively (i.e., Month 12 PP set and Month 24 PP set). The PP sets consisted of all mITT subjects who had a valid GA lesion area assessment for either Month 10 or 12 (Month 12 PP set) or have a valid GA lesion area assessment for at least one of Month 18, 20, 22, 24 (Month 24 PP set) and who follow the protocol without any major deviation(s) that could affect the primary efficacy data. A valid GA lesion area assessment is defined as a non-missing measured GA lesion area assessment at a given timepoint where at least 75% of the expected injections over the course of participation ahead of the given timepoint have been received by the subject. For example, a valid GA lesion measurement at Month 10 in the Monthly arm would be a result available at Month 10 with no more than 2 missed injections prior to the Month 10 assessment (10 scheduled before Month 10, 2 missed=80% compliance); a valid GA lesion measurement at Month 20 in the every other month (EOM) arm would be a result available at Month 20 with no more than 2 missing injections prior to the Month 20 assessment (10 scheduled before Month 20, 2 missed=80% compliance).

Efficacy analysis including primary, key secondary, secondary, and exploratory analysis will be performed primarily using the mITT set, with subjects grouped according to the treatment assigned at randomization. Available data from all randomized subjects regardless of adherence to the protocol will be included in the efficacy analyses; this includes data from subjects who discontinued study drug early but continued with study assessments. All efficacy data will be listed for the ITT set.

Unless otherwise noted, hypothesis testing and estimation of treatment effects will be performed with a mixed effect model for repeated measure (MMRM) that includes data from all three treatment arms (PM, PEOM, and Sham). The Sham arm will represent the pool of the two sham treatment groups: SM and SEOM (i.e., the two sham arms will be pooled into a single “control” group). All hypothesis tests for efficacy endpoints will be two-sided.

Unless otherwise noted, analysis of efficacy endpoints (primary, key secondary, secondary, and exploratory) in the overall population will be adjusted for the following randomization stratification factors (actual status) and baseline covariates:

For primary efficacy endpoint: presence of CNV in the fellow eye (yes; no); baseline GA lesion area (<7.5 mm² or ≥7.5 mm²).

For key secondary, and secondary endpoints: presence of CNV in the fellow eye (yes; no); baseline GA lesion area (<7.5 mm² or ≥7.5 mm²); baseline value of the endpoint.

Of note, the randomization stratification factors were assessed at the screening visit. In the event of a change in status in the GA lesion area between the screening visit and the baseline visit (i.e., GA lesion area<7.5 mm² at screening to ≥7.5 mm²) then the baseline status will be used.

### Results

DERBY (621 patients enrolled) and OAKS (637 patients enrolled) were Phase 3, multicenter, randomized, double-masked, sham-controlled studies comparing the efficacy and safety of intravitreal pegcetacoplan with sham injections in patients with GA secondary to AMD at 12 months. The primary objective of the studies was to evaluate the efficacy of pegcetacoplan in patients with GA assessed by change in the total area of GA lesions from baseline as measured by fundus autofluorescence (p-value less than 0.05).

### Efficacy

The key demographics and baseline study eye characteristics of patients in the OAKS study are shown in the following Table 2, where analyses were performed on the mITT (modified Intention-to-Treat) population. The mITT population was defined as all randomized patients who received at least 1 injection of pegcetacoplan or sham and have baseline and at least one post-baseline value of GA lesion area in the study eye. (PM, pegcetacoplan monthly; PEOM, pegcetacoplan every other month; US, United States; ROW, rest of world; GA, geographic atrophy; mm, millimeters; SD, standard deviation; NL-BCVA, normal luminance best-corrected visual acuity).

TABLE 2

| | | OAKS | | |
|---|---|---|---|---|
| | | | PEOM | Sham Pooled |
| Characteristic | | [symbol] | (N = 205) | (N = 206) |
| Age, mean (SD) | | 78.8 (7.24) | 78.1 (7.74) | 78.6 (7.26) |
| Female, n (%) | | 125 (61.9%) | 117 (57.1%) | 133 (64.6%) |
| Male, n (%) | | 77 (38.1%) | 88 (42.9%) | 73 (35.4%) |
| Geographic region | | | | |
| US, n (%) | | 147 (72.8%) | 142 (69.3%) | 147 (71.4%) |
| ROW, n (%) | | 55 (27.2%) | 63 (30.7%) | 59 (28.6%) |
| Caucasian, n (%) | | 185 (91.6%) | 189 (92.2%) | 187 (90.8%) |
| GA lesion size (mm²), mean (SD) | | 8.18 (3.893) | 8.29 (3.904) | 8.20 (3.722) |
| Square root GA lesion size (mm), mean (SD) | | 2.78 (0.682) | 2.80 (0.674) | 2.79 (0.649) |
| GA lesion size, n (%) | <7.5 mm² | 101 (50.0%) | 99 (48.3%) | 104 (50.5%) |
| GA lesion location, n (%) | Extrafoveal | 88 (42.8%) | 74 (38.1%) | 60 (29.1%) |
| GA lesion focality, n (%) | Unifocal | 59 (29.2%) | 62 (30.2%) | 68 (33.0%) |
| Intermediate/large drusen, n (%) | >20 | 93 (46.0%) | 104 (50.7%) | 103 (50.0%) |

| | | | |
|---|---|---|---|
| NL-BCVA (ETDRS letters), mean (SD) | 81.0 (15.30) | 58.2 (17.03) | 57.5 (16.57) |

[symbol]

indicates data missing or illegible when filed

While most characteristics were well-balanced, including GA lesion size and focality, there was an imbalance in location, with a greater number of extrafoveal lesions in treated arms, 36-43%, than sham, 29%. A notable difference was identified as one in which the p value was less than 0.2.

The key demographics and baseline study eye characteristics of patients in the DERBY study are shown in the following Table 3, where analyses were performed on the mITT (modified Intention-to-Treat) population. The mITT population was defined as all randomized patients who received at least 1 injection of pegcetacoplan or sham and have baseline and at least one post-baseline value of GA lesion area in the study eye. (PM, pegcetacoplan monthly; PEOM, pegcetacoplan every other month; US, United States; ROW, rest of world; GA, geographic atrophy; mm, millimeters; SD, standard deviation; NL-BCVA, normal luminance best-corrected visual acuity.)

TABLE 3

| | | DERBY | | |
|---|---|---|---|---|
| | | PM | PEOM | Sham Pooled |
| Characterstic | | (N = 201) | (N = 200) | (N = 194) |
| Age, mean (SD) | | 78.7 (6 91) | 79.2 (7.07) | 78.6 (7.29) |
| Female, n (%) | | 118 (58.7%) | 120 (60.0%) | 122 (62.9%) |
| Male, n (%) | | 83 (41.3%) | 80 (40,0%) | 72 (37.1%) |
| Geographic region | | | | |
| US, n (%) | | 142 (70.6%) | 122 (61.0%) | 122 (62.9%) |
| ROW, n (%) | | 59 (29.4%) | 78 (39.0%) | 72 (37.1%) |
| Caucasian, n (%) | | 187 (93.0%) | 185 (92.5%) | 187 (96.4%) |
| GA lesion size (mm²), mean (SD) | | 8.35 (4.182) | 8.22 (3.886) | 8.26 (4.260) |
| Square root GA lesion size (mm), mean (SD) | | 2.80 (0.723) | 2.79 (0.877) | 2.78 (0.734) |
| GA lesion size, n (%) | <7.5 mm² | 99 (49.3%) | 98 (49.0%) | 94 (48.5%) |
| GA lesion location, n (%) | Extrafoveal | 72 [symbol] | 81 (40.5%) | 73 (37.6%) |
| GA lesion focality, n (%) | Unifocal | 54 (26.9%) | 53 (26.5%) | 88 (34.0%) |
| Intermediate/Large Drusen, n (%) | >20 | 78 (38.8%) | 78 (39 0%) | [symbol] |

| | | | |
|---|---|---|---|
| NL-BCVA, mean (SD) | 59.5 (17.40) | 58.9 (15.97) | 59.1 (16.85) |

[symbol]

indicates data missing or illegible when filed

In DERBY, most baseline characteristics are well-balanced, such as GA lesion size and location. However, imbalances were noted with lesion focality and drusen. A notable difference was identified as one in which the p value was less than 0.2.

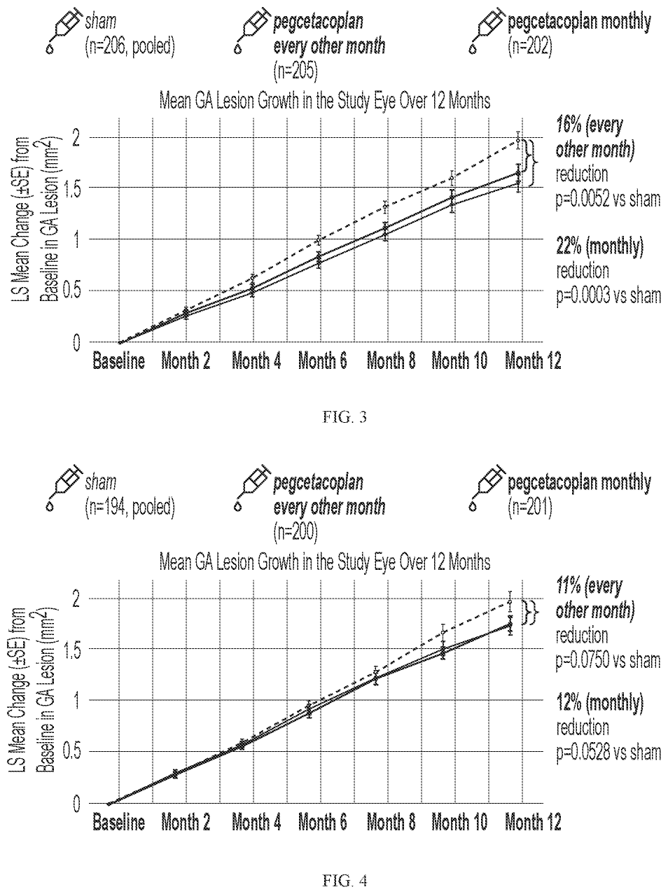

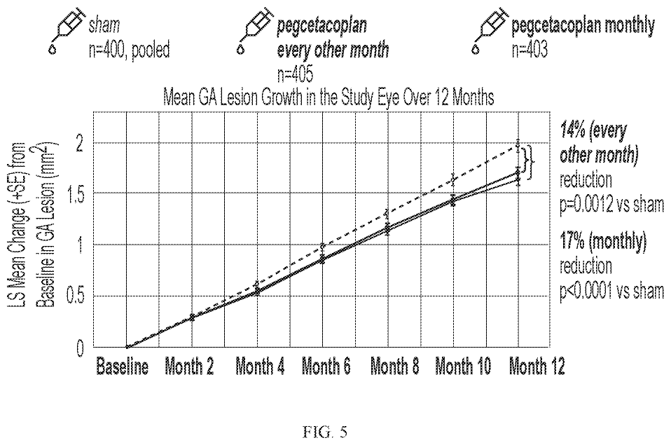

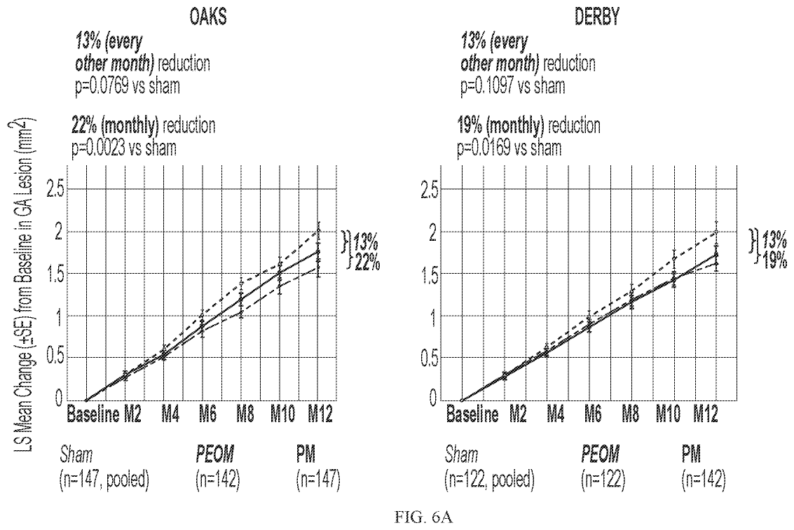

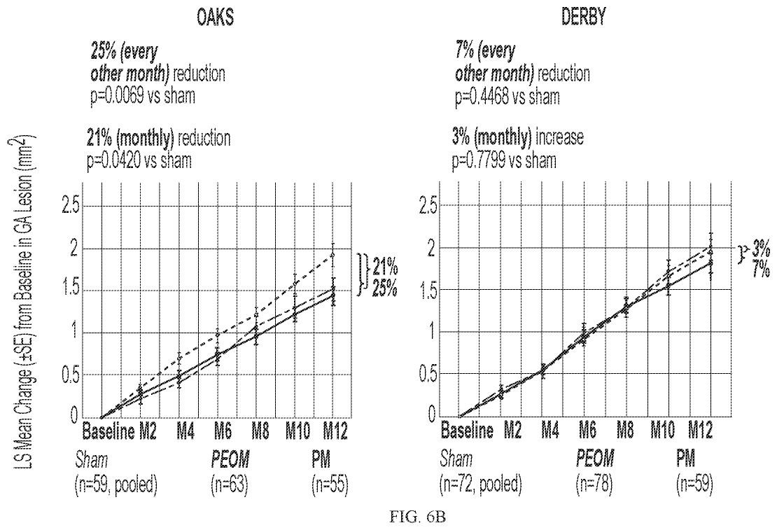

Monthly and every-other-month treatment with pegcetacoplan met the primary endpoint in OAKS, significantly reducing GA lesion growth by 22% (p=0.0003) and 16% (p=0.0052), respectively, compared to pooled sham at 12 months (see FIG. 3). DERBY did not meet the primary endpoint, showing a reduction in GA lesion growth of 12% (p=0.0528) and 11% (p=0.0750) with monthly and every-other-month treatment, respectively, compared to pooled sham at 12 months (FIG. 4). In a prespecified analysis of the combined DERBY and OAKS studies, pegcetacoplan showed a statistically significant effect with both monthly (17%, p<0.0001) and every-other-month treatment (14%, p<0.0012) compared to pooled sham at 12 months (FIG. 5). FIG. 6A shows mean change from baseline in lesions for patients in the United States in the OAKS and DERBY studies, whereas FIG. 6B shows mean change from baseline in lesions for patients in the rest of the world.

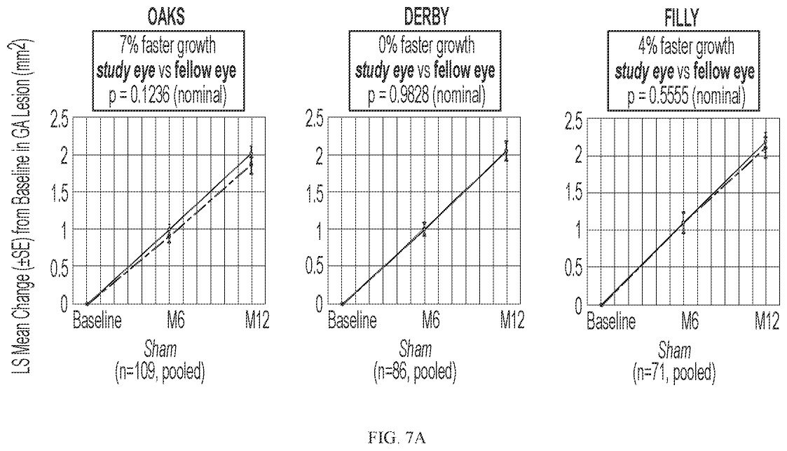

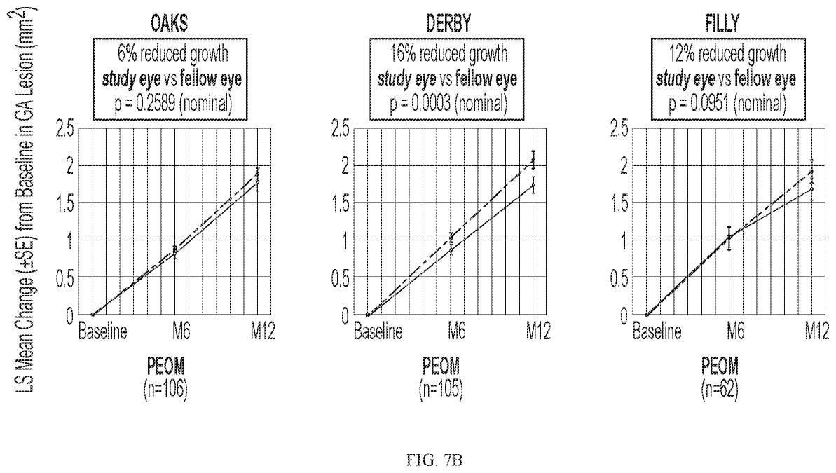

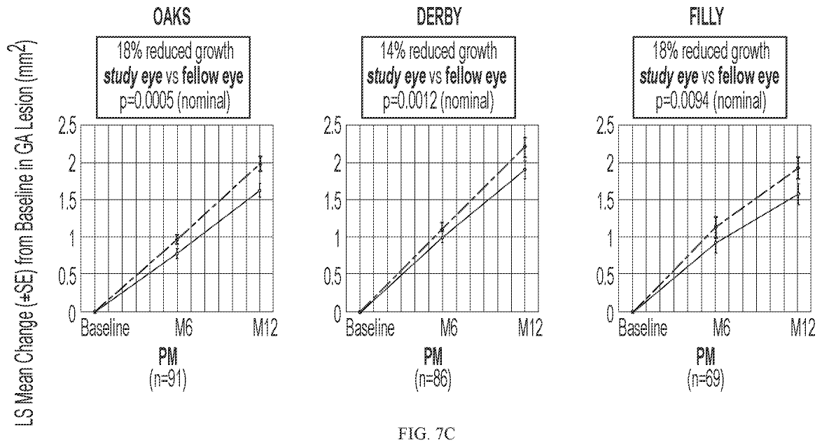

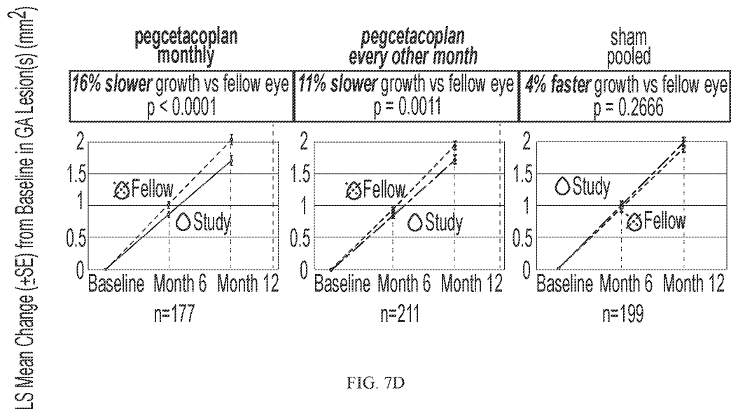

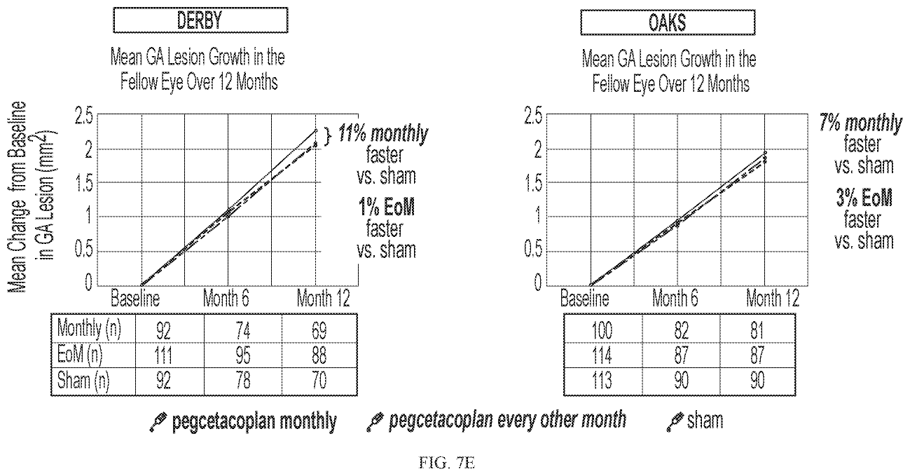

For analysis of fellow eye vs study eye lesion growth, patients with bilateral GA were included in this analysis of DERBY and OAKS. In addition, for a subject to be included, the fellow eye had to meet the following criteria: absence of CNV in the medical history; baseline GA lesion size between 2.5 and 17.5 mm²; presence of any pattern of hyperautofluorescence in the junctional zone of GA; GA not confluent with peripapillary atrophy. In the DERBY and OAKS studies, fellow eye GA lesion characteristics were similar to study eye GA lesion characteristics (e.g., majority were multifocal, and the majority were foveal-involving). For FILLY, all bilateral GA patients were included due to lower sample size. (The FILLY study was a phase 2 study that assessed change from baseline in square root GA lesion size in 80 pooled sham patients, 78 patients who received pegcetacoplan every other month, or 84 patients who received pegcetacoplan monthly. At month 12 following initiation of administrations, patients who received pegcetacoplan every other month demonstrated a 20% reduction (p=0.067 versus sham), and patients who received pegcetacoplan monthly demonstrated a 29% reduction (p=0.008 versus sham). See Liao et al., Ophthalmology 127:186-95 (2020)). As shown in FIG. 7A, sham pooled groups showed balanced lesion growth in study eyes vs. fellow eyes. FIGS. 7B and 7C show that every other month treatment (FIG. 7B) or monthly treatment (FIG. 7C) reduced lesion growth in treated study eyes vs. untreated fellow eyes in the OAKS, DERBY, and FILLY studies. In a prespecified analysis of the combined DERBY and OAKS studies, pegcetacoplan significantly slowed the growth of GA in the study eye vs. untreated fellow eye (FIG. 7D). The left-hand graph of FIG. 7D shows that monthly treatment of pegcetacoplan reduced lesion growth by 16% compared to the untreated fellow eye, and the middle graph shows that every-other-month treatment of pegcetacoplan reduced lesion growth by 11%. The graph on the right-hand side of FIG. 7D shows that in the sham group, the lesion in the study eye actually grew faster by 4% compared to the lesion in the untreated fellow eye. As shown in FIG. 7E, the underlying growth rate in the untreated fellow eye in the monthly pegcetacoplan groups was faster than the untreated fellow eye in the sham groups, by 11% in DERBY and 7% in OAKS, at month 12. For every-other-month, this disparity was less pronounced.

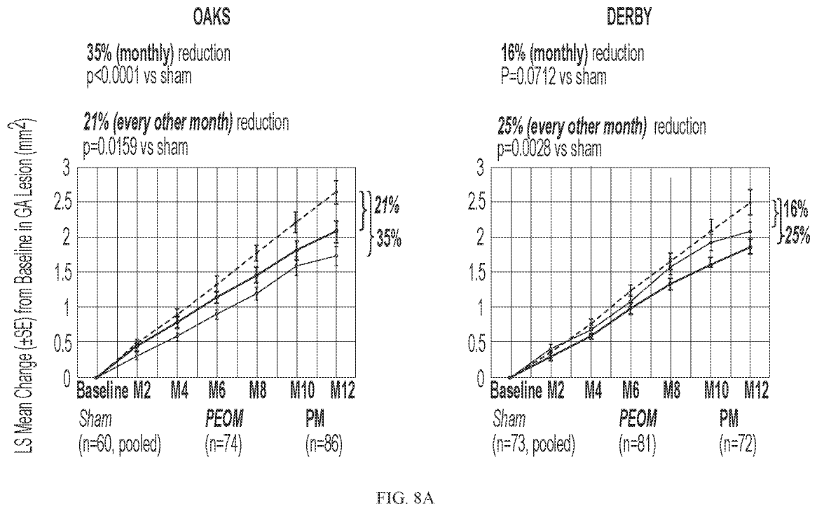

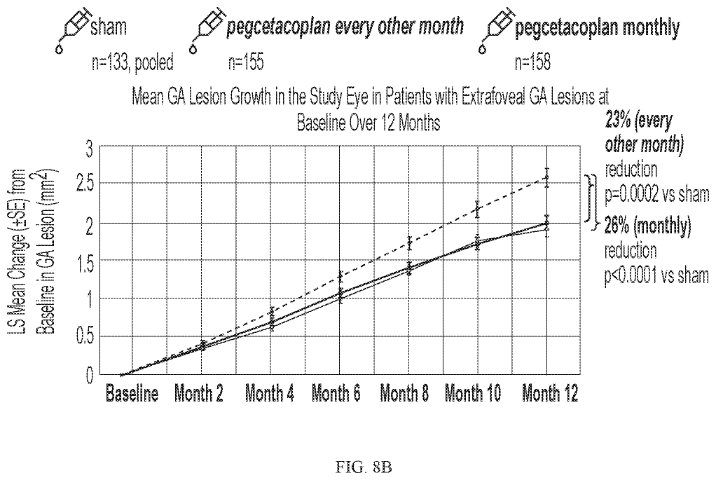

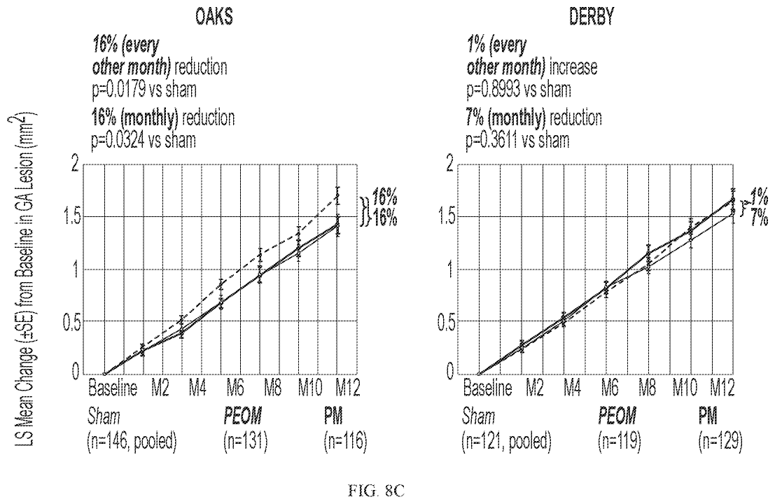

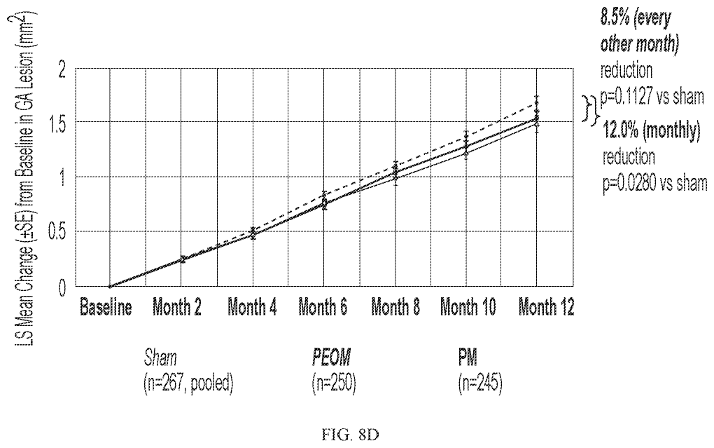

In a prespecified analysis of the combined studies, pegcetacoplan demonstrated a greater effect in patients with extrafoveal lesions at baseline. GA patients typically present first with extrafoveal lesions, which then progress toward the fovea where central vision is impacted. As shown in FIG. 8A, pegcetacoplan reduced lesion growth in patients with extrafoveal lesions in both studies. In the combined studies, pegcetacoplan decreased GA lesion growth by 26% (p<0.0001) with monthly treatment and 23% (p=0.0002) with every-other-month treatment in patients with extrafoveal lesions, in each case compared to pooled sham (FIG. 8B). As shown in FIG. 8C, pegcetacoplan reduced lesion growth in patients with subfoveal lesions in the OAKS study, and FIG. 8D shows that pegcetacoplan reduced lesion growth in patients with subfoveal lesions in the combined studies.

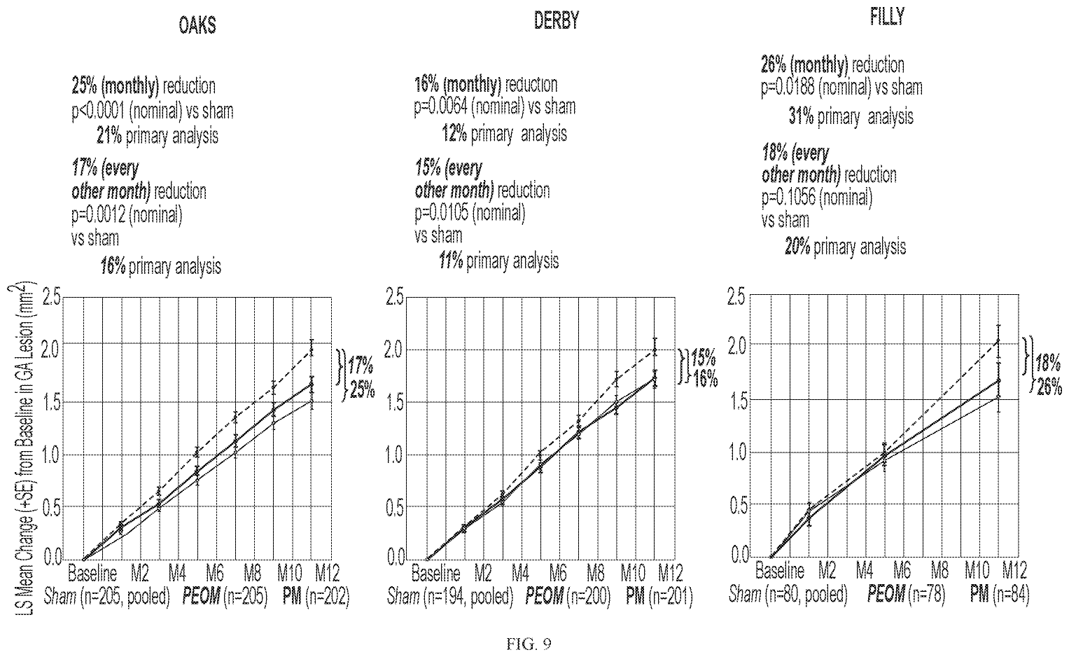

A post-hoc analysis was undertaken to examine the potential contribution of baseline characteristic imbalances on the results for the OAKS, DERBY, and FILLY studies. The following eight most relevant variables related to GA were investigated for imbalance: Study Eye Focality (Multifocal/Unifocal); Study Eye Lesion Location (subfoveal/non-subfoveal); Study Eye Lesion Size (continuous); Study Eye Pseudodrusen (Present/Absent); Study Eye Low Luminance Deficit (Continuous); Region (Derby: US/LA/ROW ex LA; Oaks: US/ROW; Filly: US/ROW); GA Laterality (Bilateral/Study Eye Only); and Study Eye Intermediate/Large Drusen (>20/Other). These eight variables were compared across the 3 treatment arms within a study via a chi-squared test or analysis of variance model and any variables with a p<0.2 from these tests was added to the common set of covariates to adjust for in this post-hoc analysis. The following four variables were identified as imbalanced: Lesion Location (Oaks)-Non-subfoveal: PM: 42.6%, PEOM: 36.1%, Sham Pooled: 29.1%; Focality (Derby)-Unifocal-PM: 26.9%, PEOM: 26.5%, Sham Pooled: 34.0%; Intermediate/Large Drusen (Filly and Derby)-Derby: >20: PM: 38.8%, PEOM: 39%, Sham Pooled: 50.5%; Filly: >20: PM: 51.2%, PEOM: 39.7%, Sham Pooled: 38.8%; Low Luminance Deficit (Filly)-Mean Letters: PM: 23.0, PEOM: 27.3, Sham Pooled: 26.5. The MMRM analysis models for the change from baseline in study eye GA lesion area in the 3 studies were re-run adjusted for these four variables as well as the interaction with visit for each of the four variables. As shown in FIG. 9, after covariate adjustment for imbalances, a convergence of the treatment effects was seen in the OAKS, DERBY, and FILLY studies.

### Safety

Pegcetacoplan was well tolerated in both Phase 3 studies. Tables 4 and 5 show the patient disposition and exposure, respectively, for the patients in the OAKS and DERBY studies.

TABLE 4
patient disposition

| | OAKS | | | DERBY | | |
|---|---|---|---|---|---|---|
| | | | Sham | | | Sham |
| | PM | PEOM | Pooled | PM | PEOM | Pooled |
| | (N = 213) | (N = 212) | (N = 212) | (N = 206) | (N = 208) | (n = 207) |
| Completed Study through | 184 (86.4%) | 190 (89.6%) | 191 (90.1%) | 183 (88.8%) | 188 (90.4%) | 179 (86.5%) |
| Month 12, n (%) | | | | | | |
| Discontinued Study prior | 29 (13.6%) | 22 (10.4%) | 21 (9.9%) | 23 (11.2%) | 20 (9.6%) | 28 (13.5%) |
| to Month 12, n (%) | | | | | | |
| Reason for discontinuation | | | | | | |
| Consent Withdrawal | 14 (6.6%) | 11 (5.2%) | 9 (4.2%) | 15 (7.3%) | 10 (4.8%) | 15 (7.2%) |
| Death | 7 (3.3%) | 3 (1.4%) | 4 (1.9%) | 5 (2.4%) | 0 | 3 (1.4%) |
| Adverse Event | 3 (1.4%) | 5 (2.4%) | 2 (0.9%) | 1 (0.5%) | 4 (1.9%) | 5 (2.4%) |
| Covid-19 Impact | 3 (1.4%) | 2 (0.9%) | 5 (2.4%) | 1 (0.5%) | 6 (2.9%) | 5 (2.4%) |
| Lost to Follow-up | 2 (0.9%) | 1 (0.5%) | 1 (0.5%) | 1 (0.5%) | 0 | 0 |

TABLE 5
exposure

| | OAKS | | | DERBY | | |
|---|---|---|---|---|---|---|
| | | | Sham | | | Sham |
| | PM | PEOM | Pooled | PM | PEOM | Pooled |
| | (N = 213) | (N = 212) | (N = 212) | (n = 201) | (n = 200) | (n = 194) |
| Total number of injections | 2056 | 1103 | 1597 | 2058 | 1063 | 1496 |
| received | | | | | | |
| Total number of missed | 252 | 86 | 203 | 281 | 109 | 184 |
| injections, n (%) | (9.2%) | (7.2%) | (11.3%) | (12.0%) | (9.3%) | (11.0%) |
| Mean number of | 10.2/12, 85% | 5.4/6, 90% | 7.8/9, 87% | 10.2/12, 85% | 5.3/6, 88% | 7.7/9, 86% |
| injections/patient, n. % | (2.31) | (1.2) | (3.08) | (2.24) | (1.05) | (3.02) |
| (SD) | | | | | | |
| Mean duration^{a} of | 334.5 | 337.8 | 332.3 | 335.3 | 338.0 | 335.6 |
| treatment, days (SD) | (65.40) | (63.21) | (64.05) | (60.98) | (56.52) | (60.09) |

^{a}Duration of treatment in monthly group was (date of last injection + 30 days) - date of first injection + 1; EOM group was (date of last injection + 60 days) − date of first injection + 1. Duration of treatment was truncated to a patient's early termination date, Month 12 cutoff date, or study completion date, as appropriate. Approximately half of missed injections were
attributed to COVID-19.

Table 6 lists the overall TEAEs observed for the OAKS and DERBY studies:

TABLE 6
TEAEs observed for OAKS and DERBY

| | OAKS | | | DERBY | | |
|---|---|---|---|---|---|---|
| | PM | PEOM | Sham Pooled | PM | PEOM | Sham Pooled |
| | (N = 213) | (N = 212) | (N = 211) | (N = 206) | (N = 208) | (N = 206) |
| All TEAEs, n (%) | 170 (79.8%) | 160 (75.5%) | 154 (73.0%) | 158 (76.7%) | 155 (74.5%) | 145 (70.4%) |
| Total events, M | 751 | 721 | 666 | 708 | 600 | 530 |
| Ocular TEAEs in study eye | 108 (50.7%) 256 | 97 (45.8%) 208 | 74 (35.1%) 159 | 100 (48.5%) 227 | 91 (43.8%) 162 | 72 (35.0%) 126 |
| Patients, n (%) M | | | | | | |
| Non-ocular TEAEs | 136 (63.8%) 390 | 127 (59 9%) 412 | 125 (59.2%) 413 | 127 (61.7%) 396 | 110 (52.9%) 339 | 112 (54.4%) 329 |
| Patients, n (%) M | | | | | | |
| Serious ocular TEAEs in the | 3 (1.4%) 3 | 4 (1.9%) 4 | 0 | 1 (0.5%) 1 | 0 | 2 (1.0%) 2 |
| study eye, n (%) M | | | | | | |
| Optic ischemic | 1 (0.5%) 3 | 0 | 0 | 0 | 0 | 0 |
| neuropathy | | | | | | |
| Papilledema | 1 (0.5%) 3 | 0 | 0 | 0 | 0 | 0 |
| Retinal detachment | 0 | 1 (0.5%) 1 | 0 | 0 | 0 | 0 |
| Endophthalmitis [symbol] | 1 (0.5%) 3 | 3 (1.4%) 3 | 0 | 0 | 0 | 0 |
| Vitritis | 0 | | 0 | 1 (0.5%) 1 | 0 | 0 |
| Dry AMD | 0 | | 0 | 0 | 0 | 1 (0.5%) 1 |
| Macular hole | 0 | 0 | 0 | 0 | 0 | 1 (0.5%) 1 |

[symbol]

[symbol]

indicates data missing or illegible when filed

Table 7 lists the most common ocular AEs in the study eye (>5%) for the OAKS and DERBY studies.

TABLE 7
most common ocular AEs

| | OAKS | | |
|---|---|---|---|
| | PM | PEOM | Sham pooled |
| | (N = 213) | (N = 212) | (N = 211) |
| Number of patients with ≥1 ocular TEAE in the study eye, n (%) M | 108 (50.7%) 256 | 97 (45.8) 208 | 74 (35.1) 159 |
| Ocular TEAEs in Study Eye in ≥5% of pegcetacoplan patients, n (%) M | | | |
| Conjunctival haemorrhage | 18 (8.5%) 30 | 13 (6.1%) 16 | 6 (2.8%) 6 |
| Eye Pain | 15 (7.0%) 17 | 12 (5.7%) 14 | 11 (5.2%) 14 |
| Vitreous Floaters | 9 (4.2%) 10 | 15 (7.1%) 20 | 1 (0.5%) 1 |
| | DERBY | | |
| | PM | PEOM | Sham pooled |
| | (N = 206) | (N = 208) | (N = 206) |
| Number of patients with ≥1 ocular TEAE in the study eye, n (%) M | 100 (48.5%) 227 | 91 (43.8) | 72 (35.0%) 126 |
| Ocular TEAEs in Study Eye in >5% of peacetacoplan patients, n (%) M | | | |
| Vitreous floaters | 18 (8.7%) 22 | 4 (1.9%) 5 | 3 (1.5%) 3 |

[symbol]

[symbol]

indicates data missing or illegible when filed

In assessing onset of exudations, some of the key relevant inclusion/exclusion criteria from the DERBY and OAKS studies included the following. For the fellow eye, there were no exclusion criteria, and a history of fellow eye exudative AMD (eAMD) was not exclusionary. Across the OAKS and DERBY study arms, 18-21% of patients had fellow eye CNV present at baseline. For the study eye, any history of or evidence of active eAMD was exclusionary, whereas patients with subclinical macular neovascularization (MNV) either by double layer sign (DLS) or optical coherence tomography angiography (OCT-A) were not excluded. Across the OAKS and DERBY study arms, 14-20% of patients had study eye DLS present at baseline. Table 8 lists the characteristics of exudative AMD in the study eye (events included preferred terms of choroidal neovascularization and neovascular AMD).

TABLE 8
Characteristics of exudative AMD in study eyea

| | OAKS | | | DERBY | | |
|---|---|---|---|---|---|---|
| | PM | PEOM | Sham Pooled | PM | PEOM | Sham Pooled |
| Patients with investigator-determined | 11 | 10 | 3 | 14 | 7 | 7 |
| new onset exudative AMD, n (%) | (5.2%) | (4.7%) | (1.4%) | (6.8%) | (3.4%) | (3.4%) |
| Cases of MNV (FA) detected by | 2 | 3 | 5 | 0 | 1 | 1 |
| reading center but not reported by | | | | | | |
| Investigator as AE | | | | | | |
| Sum of investigator-determined eAMD | 13 | 13 | 8 | 14 | 8 | 8 |
| and reading center cases not reported | (6.1%) | (6.1%) | (3.8%) | (6.8%) | (3.8%) | (3.9%) |
| by investigators | | | | | | |

[symbol]

There were no significant changes in BOVA; findings in OCT were as expected [symbol]
Six out of 52 investigator-determined cases were not confirmed by the reading center [symbol]
All reading center determined cases that were not reported by investigators as AEs were occult [symbol]
All cases with available FA showed occult MNV except for 2 classical cases in DERBY [symbol]
Combined investigator-determined eAMD rates across the studies: 6.0% PM, 4.1% PEOM, 2.4% Sham [symbol]
Including reading center detected cases not reported by investigators: 6.4% PM, 5.0% PEOM, 3.8% Sham

[symbol]

[symbol]

indicates data missing or illegible when filed

Investigator-determined e-AMD was reported in 5.2 and 6.8% of patients in the PM arms, 4.7 and 3.4% in PEOM arms and 1.4 and 3.4% of the sham arms. The eAMD rates in Table 8 include all eAMD cases reported as AEs by investigators. Six of the 52 investigator-determined cases (12%) were not confirmed by the reading center (stated differently, 46/52 cases or 88% of investigator-determined cases were confirmed by the reading center). DERBY: PM: 3 cases, PEOM 0, Sham 1. OAKS: PM 2 cases, PEOM 0, Sham 0. In addition, 6 pegcetacoplan treated and 6 sham eyes, for a total of 12 additional eyes, had MNV that was detected by the masked reading center (by FA) that was not reported by the investigator at month 12. Table 9 lists rate of eAMD by fellow eye CNV status, and Table 10 lists treatment of exudative AMD in study eye.

TABLE 9
Rate of eAMD by fellow eye CNV status

| | OAKS | | | DERBY | | |
|---|---|---|---|---|---|---|
| | PM | PEOM | Sham Pooled | PM | PEOM | Sham Pooled |
| | (N = 213) | (N = 212*) | (N = 211) | (N = 206) | (N = 208) | (N = 206) |
| With fellow eye CNV | | | | | | |
| n/N | 1/45 | 3/39 | 3/44 | 5/39 | 1/42 | 4/42 |
| % | 2.2% | 7.7% | 6.8% | 12.8% | 2.4% | 9.5% |
| Without fellow eye CNV, n/N (%) | | | | | | |
| N/N | 10/168 | 7/172 | 0/167 | 9/167 | 6/166 | 3/164 |
| % | 6% | 4.1% | 0% | 5.4% | 3.6% | 1.8% |

[symbol]

[symbol]

indicates data missing or illegible when filed

TABLE 10
Treatment of exudative AMD in study eye^{a}

| | OAKS | | | DERBY | | |
|---|---|---|---|---|---|---|
| | PM | PEOM | Sham Pooled | PM | PEOM | Sham Pooled |
| Patients with new onset exudative | 11 | 10 | 3 | 14 | 7 | 7 |
| AMD, n (%) | (5.2%) | (4.7%) | (1.4%) | (6.8%) | (3.4%) | (3.4%) |
| Patients with concomitant anti-VEGF | 11 | 10 | 3 | 14 | 6 | 5 |
| Injections in Study Eye, n (%) | (100%) | (100%) | (100%) | (100%) | (85.7%) | (71.4%) |

None of the cases detected by the reading center but not reported by investigators were treated with anti-VEGF
Patients continue to receive study medication according to their randomized arm

[symbol]

[symbol]

indicates data missing or illegible when filed

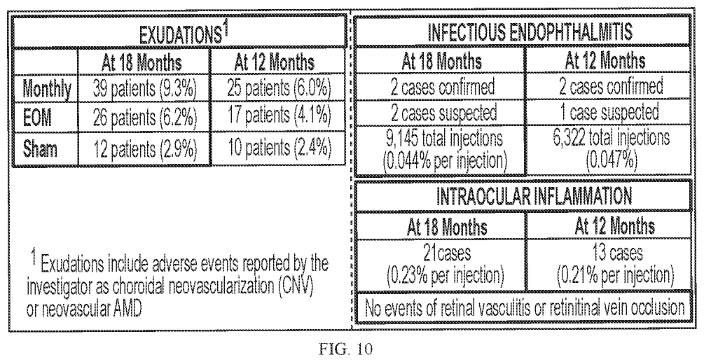

In summary, the pooled rate of new-onset exudations was 6.0% of patients in the monthly pegcetacoplan groups, 4.1% in the every-other-month pegcetacoplan groups, and 2.4% in the sham groups (FIG. 10). Most patients who developed exudations were treated with approved anti-VEGF therapies. Two cases of confirmed infectious endophthalmitis and one case of suspected infectious endophthalmitis were observed in the study eye out of a total of 6,332 injections (0.047%). Thirteen events of intraocular inflammation were also observed in the studies (0.21% per injection), with the majority of cases being mild and 10 of 13 patients resuming administration without subsequent recurrence of intraocular inflammation. No events of retinal vasculitis or retinal vein occlusion were observed. There were no clinically relevant changes in vision for patients who developed infectious endophthalmitis or intraocular inflammation.

### Example 2-18 Month Results from Phase III Studies

This Example describes longer-term data from the Phase 3 DERBY and OAKS studies described in Example 1. These data showed that intravitreal pegcetacoplan continued to reduce geographic atrophy (GA) lesion growth and demonstrate a favorable safety profile at month 18 for the treatment of GA secondary to age-related macular degeneration (AMD).

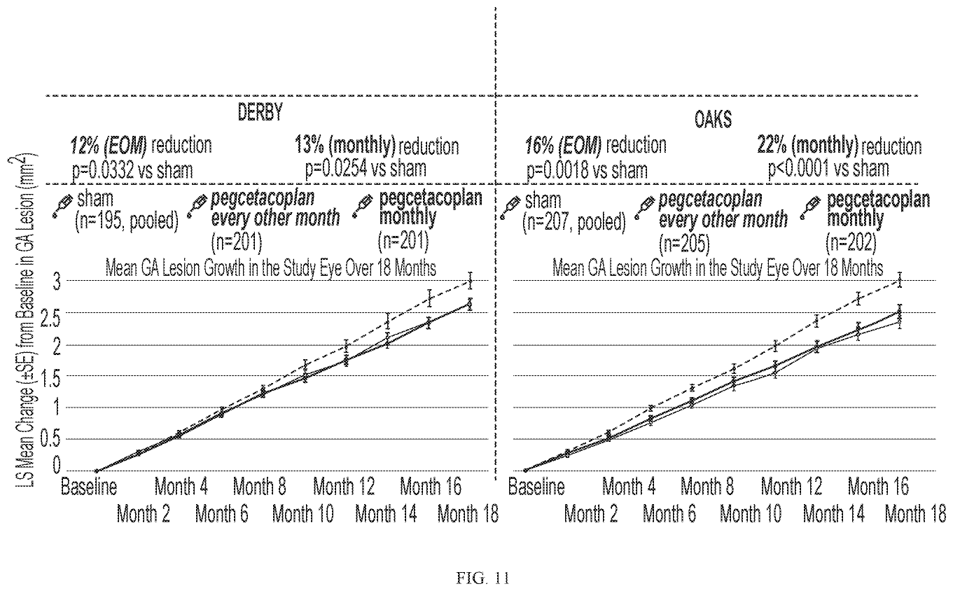

In the longer-term analysis of the primary endpoint, pegcetacoplan continued to reduce GA lesion growth compared to pooled sham at month 18 (all p values were nominal). In OAKS, pegcetacoplan reduced GA lesion growth with both monthly (22%; p<0.0001) and every-other-month treatment (16%; p=0.0018) (FIG. 11). In DERBY, pegcetacoplan reduced GA lesion growth with both monthly (13%; p-0.0254) and every-other-month treatment (12%; p=0.0332) (FIG. 11).

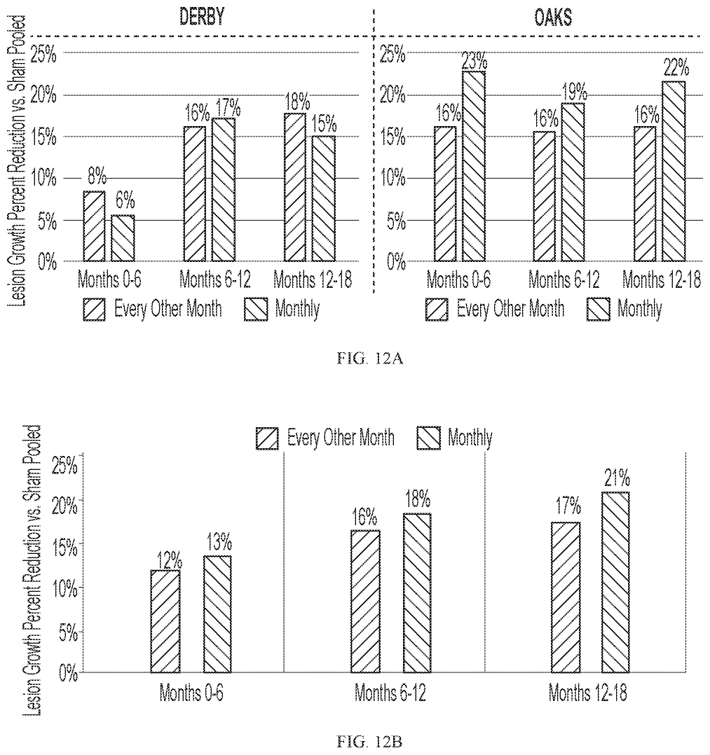

Pegcetacoplan demonstrated marked improvements in DERBY during months 6-12 with reductions of 17% with monthly and 16% with every-other-month treatment compared to months 0-6, and the treatment effects were sustained through month 18 (FIG. 12A). The treatment effects observed in DERBY were comparable with OAKS during months 6-18 (FIG. 12A).

The reduction in GA lesion growth improved with monthly pegcetacoplan treatment from 13% to 21% from months 0-6 to months 12-18 (FIG. 12B). The reduction in GA lesion growth improved with every-other-month pegcetacoplan treatment from 12% to 17% from months 0-6 to months 12-18.

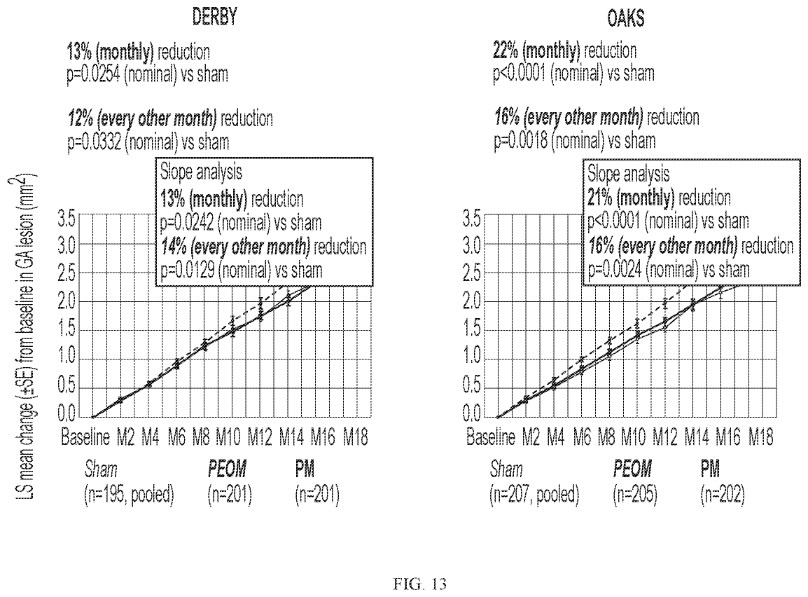

Both DERBY and OAKS demonstrated reduced GA lesion growth versus sham at month 18 using a slope analysis (FIG. 13).

Tables 11 and 12 show the patient disposition at 18 months and exposure at 18 months, respectively, for the patients in the OAKS and DERBY studies.

TABLE 11
Patient Disposition at 18 Months

| | DERBY | | | OAKS | | |
|---|---|---|---|---|---|---|
| | | | Sham | | | Sham |
| | PM | PEOM | Pooled | PM | PEOM | Pooled |
| | (N = 206) | (N = 208) | (N = 207) | (N = 213) | (N = 212) | (N = 212) |
| Completed study through | 167 (81.1%) | 176 (84.6%) | 172 (83.1%) | 165 (77.5%) | 179 (84.4%) | 172 (81.1%) |
| Month 18, n (%) | | | | | | |
| Discontinued study prior | 39 (18.9%) | 32 (15.4%) | 35 (16.9%) | 48 (22.5%) | 33 (15.6%) | 40 (18.9%) |
| to Month 18, n (%) | | | | | | |
| Reason for discontinuation, n (%) | | | | | | |
| Consent withdrawal | 24 (11.7%) | 13 (6.3%) | 18 (8.7%) | 22 (10.3%) | 14 (6.6%) | 14 (6.6%) |
| Death | 6 (2.9%) | 4 (1.9%) | 6 (2.9%) | 12 (5.6%) | 7 (3.3%) | 7 (3.3%) |
| Adverse event | 3 (1.5%) | 4 (1.9%) | 5 (2.4%) | 6 (2.8%) | 4 (1.9%) | 3 (1.4%) |
| COVID-19 impact | 3 (1.5%) | 9 (4.3%) | 6 (2.9%) | 5 (2.3%) | 3 (1.4%) | 11 (5.2%) |
| Lost to follow-up | 1 (0.5%) | 2 (1.0%) | 0 | 3 (1.4%) | 4 (1.9%) | 4 (1.9%) |

TABLE 12
Exposure at 18 Months

| | DERBY | | | OAKS | | |
|---|---|---|---|---|---|---|
| | | | Sham | | | Sham |
| | PM | PEOM | Pooled | PM | PEOM | Pooled |
| | (N = 201) | (N = 201) | (N = 195) | (N = 202) | (N = 205) | (N = 207) |
| Total number of injections | 2947 | 1553 | 2181 | 2991 | 1610 | 2321 |
| received | | | | | | |
| Total number of missed | 418 | 176 | 298 | 359 | 127 | 303 |
| injections | | | | | | |
| Mean number of | 14.7 | 7.7 | 11.2 | 14.8 | 7.9 | 11.2 |
| injections/patient, n (SD) | (4.03) | (1.87) | (4.70) | (4.08) | (1.97) | (4.74) |
| Mean duration^{a} of | 482.5 | 496.1 | 492.2 | 485.5 | 491.1 | 489.7 |
| treatment, days (SD) | (117.63) | (105.89) | (115.21) | (120.49) | (120.20) | (113.78) |
| Mean compliance, % | 87.0% | 89.5% | 88.8% | 88.5% | 92.4% | 88.6% |

In Table 12, the modified intent-to-treat population was used for the analysis, defined as all randomized patients who received at least 1 injection of pegcetacoplan or sham and had baseline and at least 1 post-baseline value of GA lesion area in the study eye. ªDuration of treatment in monthly group was (date of last injection+30 days)-date of first injection+1; EOM group was (date of last injection+60 days)-date of first injection+1. Duration of treatment was truncated to a patient's early termination date, Month 18 cutoff date, or study completion date, as appropriate. Compliance (%) was calculated as the number of injections administered divided by the number of scheduled injections up to completion or discontinuation of study treatment×100.

At month 18, pegcetacoplan continued to demonstrate a favorable safety profile, consistent with safety at 12 months and longer-term exposure to intravitreal injections (FIG. 10). The rate of infectious endophthalmitis was 0.044% per injection, and the rate of intraocular inflammation was 0.23% per injection. No events of retinal vasculitis or retinal vein occlusion were observed. For DERBY, patients with study eye investigator-determined new-onset exudative AMD was 11.7% (24/206 patients), 5.3% (11/208 patients), and 3.9% (8/206 patients) in the pegcetacoplan monthly, every-other-month, and sham groups, respectively. For OAKS, patients with study eye investigator-determined new-onset exudative AMD was 7.5% (16/213 patients), 7.1% (15/212 patients), and 1.9% (4/211*patients) in the pegcetacoplan monthly, every-other-month, and sham groups, respectively (*one patient in OAKS had CNV on medical history in study eye and was not counted in the denominator for this analysis; 211 patients were at risk of new-onset eAMD). The rate of new-onset exudations for the combined studies at month 18 was 9.5% (40/419 patients), 6.2% (26/420 patients), and 2.9% (12/417 patients) in the pegcetacoplan monthly, every-other-month, and sham groups, respectively. For the combined studies, investigator-reported events of eAMD through month 18 when CNV was present in the fellow eye was 9.5% (8/84 patients), 9.9% (8/81 patients), and 8.1% (7/86 patients) in the pegcetacoplan monthly, every-other-month, and sham groups, respectively. For the combined studies, investigator-reported events of eAMD through month 18 when CNV was absent in the fellow eye was 9.6% (32/335 patients), 5.3% (18/338 patients), and 1.5% (5/331 patients) in the pegcetacoplan monthly, every-other-month, and sham groups, respectively. For the combined studies, investigator-reported events of eAMD through month 18 when double-layer sign (DLS) was present in the study eye was 10.1% (8/79 patients), 8.2% (6/73 patients), and 0% (0/63 patients) in the pegcetacoplan monthly, every-other-month, and sham groups, respectively. For the combined studies, investigator-reported events of eAMD through month 18 when double-layer sign (DLS) was absent in the study eye was 9.5% (32/336patients), 5.8% (20/345 patients), and 3.2% (11/349 patients) in the pegcetacoplan monthly, every-other-month, and sham groups, respectively. Table 13 lists characteristics of investigator-reported eAMD events through month 18, and Table 14 lists cases of eAMD in fellow eyes through 18 months. Table 15 lists overall TEAEs at 18 months.

TABLE 13
Characteristics of investigator-reported eAMD events through month 18

| | PM | PEOM | Sham Pooled |
|---|---|---|---|
| COMBINED STUDIES | (N = 26) | (N = 21) | (N = 11) |
| CNV type on FA at eAMD study visit | | | |
| Classic, n (%) | 1 (3.8%) | 1 (4,8%) | 0 |
| Occult, n (%) | 22 (84.6%) | 20 (95.2%) | 10 (90.9%) |
| Classic + occult, n (%) | 0 | 0 | 0 |
| Active leakage with low likelihood of CNV, n (%) | 3 (11.5%) | 0 | 1 (9.1%) |

Table includes events with available reading center determination of CNV type on FA at time of eAMD study visit
All patients had evaluable SD-OCT at time of eAMD study visit; majority of events showed no subretinal fluid

TABLE 14
Cases of eAMD in fellow eyes through 18 months

| | OAKS | | | DERBY | | |
|---|---|---|---|---|---|---|
| | | | Sham | | | Sham |
| | PM | PEOM | Pooled | PM | PEOM | Pooled |
| | (N = 213) | (N = 212) | (N = 211) | (N = 206) | (N = 206) | (N = 206) |
| Patients without baseline | 168 | 173 | 167 | 167 | 166 | 164 |
| fellow eye CNV, N | | | | | | |
| Patients with new onset | 8 (4.8%) | 5 (2.9%) | 7 (4.2%) | 4 (2.4%) | 8 (4.8%) | 6 (3.7%) |
| fellow eye eAMD, n (%) | | | | | | |

| | PM | PEOM | Sham Pooled |
|---|---|---|---|
| COMBINED STUDIES | (N = 419) | (N = 420) | (N = 417) |
| Patients without baseline fellow eye CNV, N | 335 | 339 | 331 |
| Investigator-determined new-onset eAMD, n (%) | 12 (3.6%) | 13 (3.8%) | 13 (3.9%) |

TABLE 15
Overall TEAEs at 18 months

| | DERBY | | | OAKS | | |
|---|---|---|---|---|---|---|
| | | | Sham | | | Sham |
| | PM | PEOM | Pooled | PM | PEOM | Pooled |
| | (N = 206) | (N = 208) | (N = 206) | (N = 213) | (N = 212) | (N = 211) |
| All TEAEs, n (%) | 170 (82.5%) | 171 (82.2%) | 164 (79.6%) | 185 (86.9%) | 177 (83.5%) | 169 (80.1%) |
| Total events, | 993 | 835 | 734 | 1037 | 965 | 893 |
| M | | | | | | |
| Ocular TEAEs in | | | | | | |
| study eye | | | | | | |
| Patients, n (%) | 118 (57.3%) | 99 (47.6%) | 84 (40.8%) | 126 (59.2%) | 114 (53.8%) | 88 (41.7%) |
| M | 308 | 216 | 151 | 325 | 270 | 199 |
| Non-ocular TEAEs | | | | | | |
| Patients, n (%) | 148 (71.8%) | 132 (63.5%) | 131 (63.6%) | 152 (71.4%) | 158 (74.5%) | 144 (68.2%) |
| M | 567 | 495 | 485 | 573 | 567 | 559 |

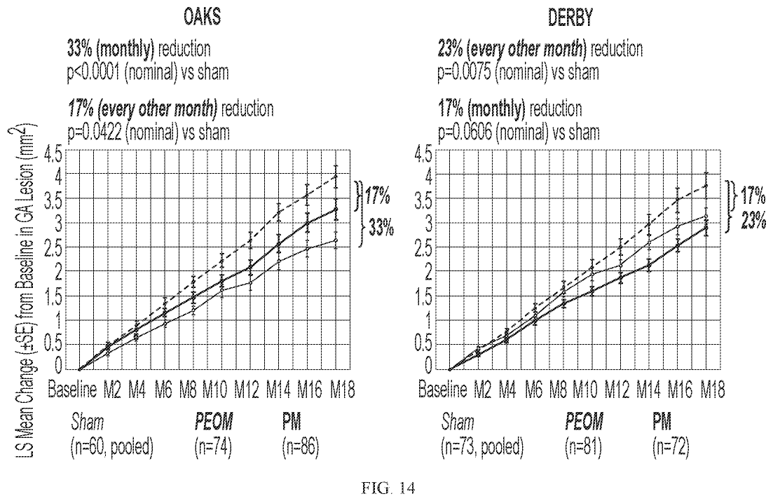

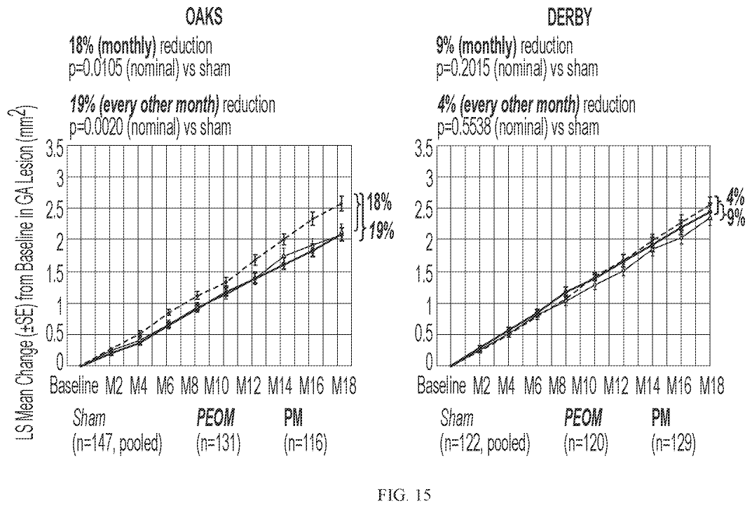

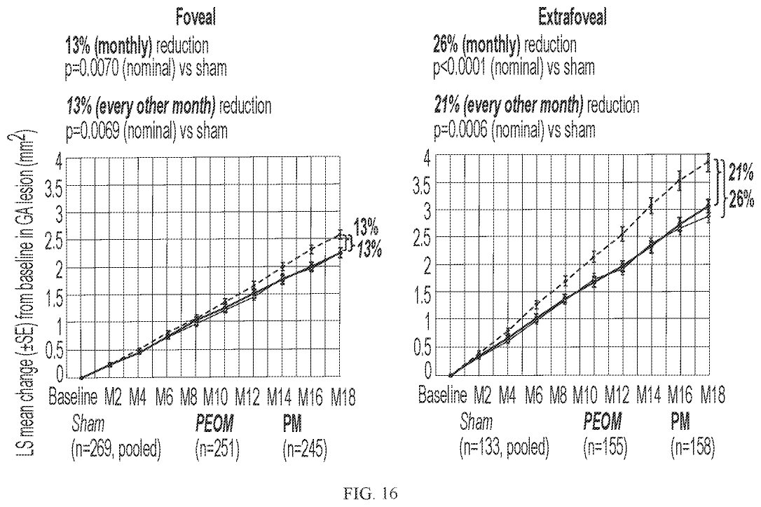

In the longer-term analysis of the primary endpoint, pegcetacoplan continued to show a robust reduction in GA lesion growth in patients with extrafoveal lesions and an improved effect in patients with foveal lesions compared to pooled sham at month 18 (all p-values were nominal). GA typically presents first with extrafoveal lesions, which then progress toward the fovea where central vision is impacted. Pegcetacoplan reduced extrafoveal GA lesion growth in OAKS by 33% (p<0.0001) and 17% (p-0.0422) with both monthly and every-other-month treatment, respectively, and in DERBY by 17% (p=0.0606) and 23% (p=0.0075), respectively (see FIG. 14). Pegcetacoplan reduced foveal GA lesion growth in OAKS by 18% (p=0.0105) and 19% (p=0.0020) with both monthly and every-other-month treatment, respectively, and in DERBY by 9% (p=0.2015) and 4% (p=0.5538), respectively (see FIG. 15). In the combined studies, pegcetacoplan reduced extrafoveal GA lesion growth by 26% (p<0.0001) and 21% (p=0.0006) with both monthly and every-other-month treatment, respectively, and reduced foveal lesion growth by 13% (p=0.0070) and 13% (p-0.0069), respectively (see FIG. 16).

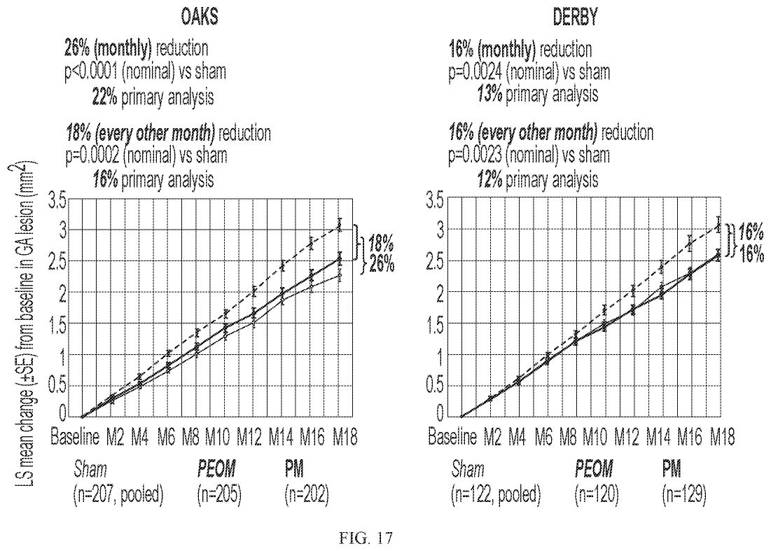

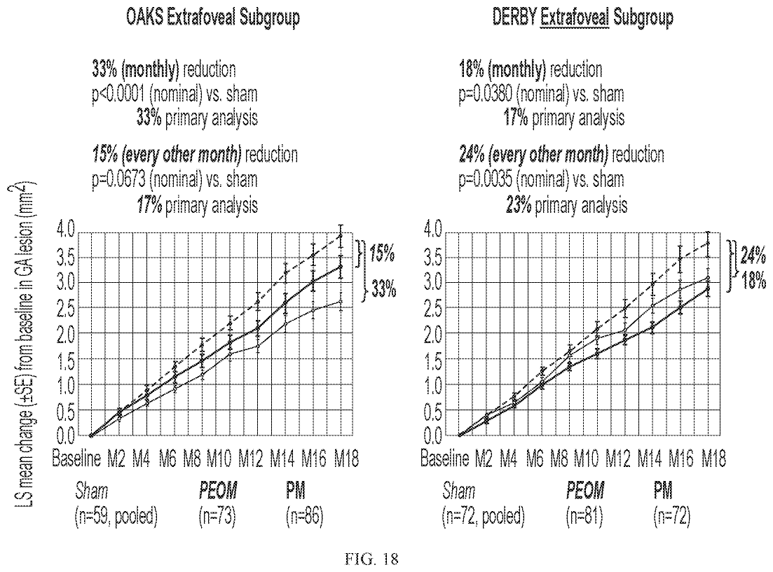

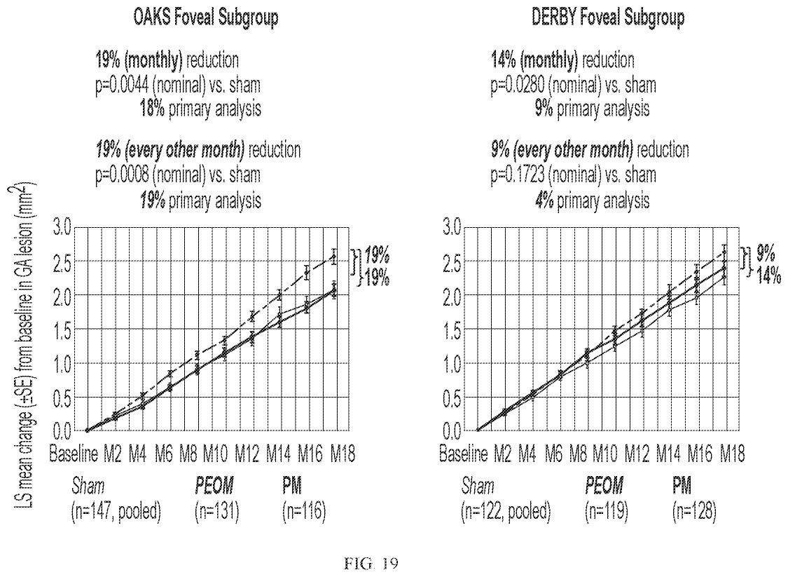

The converging treatment effect of pegcetacoplan in the OAKS and DERBY studies in covariate-adjusted post-hoc analysis described in Example 1 continued at month 18. Pegcetacoplan reduced GA lesion growth in OAKS by 26% (p<0.0001) and 18% (p=0.0002) with both monthly and every-other-month treatment, respectively, and in DERBY by 16% (p=0.0024) and 16% (p=0.0023), respectively (see FIG. 17). Pegcetacoplan reduced extrafoveal GA lesion growth in OAKS by 33% (p<0.0001) and 15% (p=0.0673) with both monthly and every-other-month treatment, respectively, and in DERBY by 18% (p=0.0380) and 24% (p=0.0035), respectively (see FIG. 18). Pegcetacoplan reduced foveal GA lesion growth in OAKS by 19% (p=0.0044) and 19% (p=0.0008) with both monthly and every-other-month treatment, respectively, and in DERBY by 14% (p=0.0280) and 9% (p=0.1723), respectively (see FIG. 19).

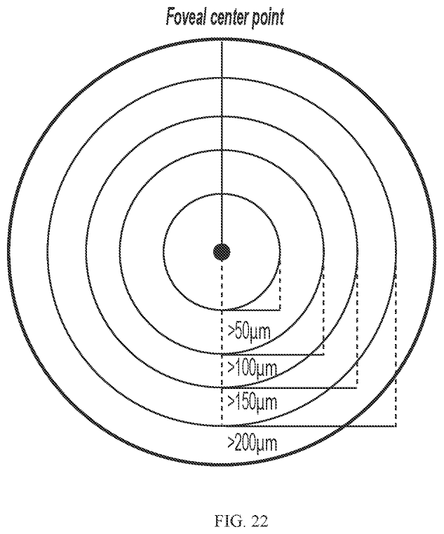

Additional subgroup analyses were performed based on lesion distance from foveal center point. An analysis was performed using the following cutoffs from the foveal center point: ≤250 microns; and >250 microns. Additional subgroups were analyzed based on different lesion distances from foveal center point, as depicted in FIG. 22: >50 microns; >100 microns; >150 microns; and >200 microns.

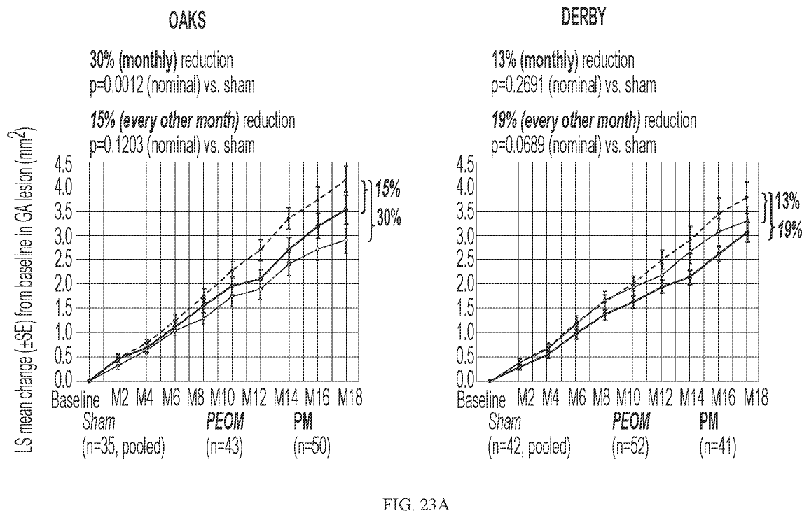

As shown in FIG. 23A, in patients from OAKS having a distance from lesion to foveal center point in the study eye of >250 microns, a 30% reduction (p=0.0012) versus sham was seen with monthly treatment, and a 15% reduction (p=0.1203) versus sham was seen with every other month treatment. In patients from DERBY having a distance from lesion to foveal center point in the study eye of >250 microns, a 13% reduction (p=0.2691) versus sham was seen with monthly treatment, and a 19% reduction (p=0.0689) versus sham was seen with every other month treatment. As shown in FIG. 23B, in patients from OAKS having a distance from lesion to foveal center point in the study eye of ≤250 microns, a 22% reduction (p=0.0002) versus sham was seen with monthly treatment, and a 19% reduction (p=0.0008) versus sham was seen with every other month treatment. In patients from DERBY having a distance from lesion to foveal center point in the study eye of ≤250 microns, a 12% reduction (p=0.0490) versus sham was seen with monthly treatment, and a 12% reduction (p=0.0724) versus sham was seen with every other month treatment.

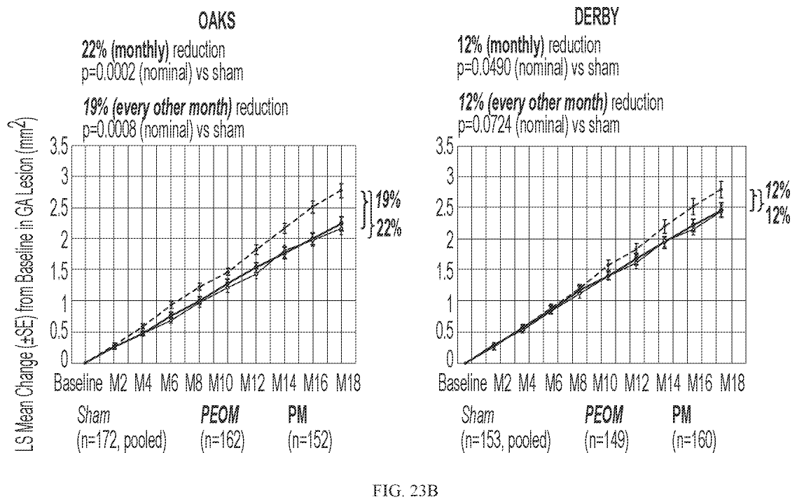

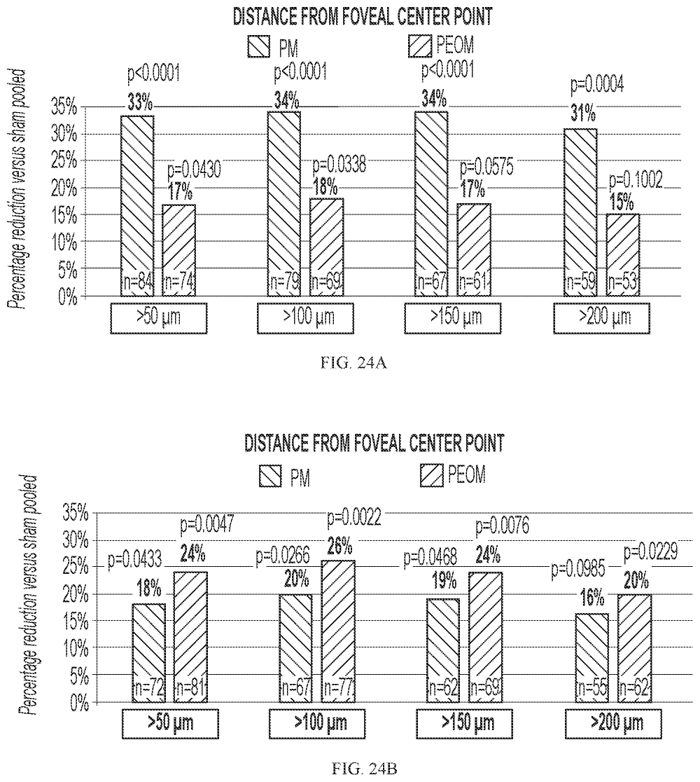

Both OAKS and DERBY demonstrated consistent efficacy with increasing distance from foveal center point. As shown in FIG. 24A, for patients from OAKS and treated monthly, patients having a distance from lesion to foveal center point in the study eye of >50 microns showed a 33% reduction (p<0.0001) versus sham pooled; patients having a distance from lesion to foveal center point in the study eye of >100 microns showed a 34% reduction (p<0.0001) versus sham pooled; patients having a distance from lesion to foveal center point in the study eye of >150 microns showed a 34% reduction (p<0.0001) versus sham pooled; and patients having a distance from lesion to foveal center point in the study eye of >200 microns showed a 34% reduction (p<0.0001) versus sham pooled (distances as shown in FIG. 22). For patients from OAKS and treated every other month, patients having a distance from lesion to foveal center point in the study eye of >50 microns showed a 17% reduction (p=0.0430) versus sham pooled; patients having a distance from lesion to foveal center point in the study eye of >100 microns showed a 18% reduction (p=0.0338) versus sham pooled; patients having a distance from lesion to foveal center point in the study eye of >150 microns showed a 17% reduction (p=0.0575) versus sham pooled; and patients having a distance from lesion to foveal center point in the study eye of >200 microns showed a 15% reduction (p=0.1002) versus sham pooled (FIG. 24A). As shown in FIG. 24B, for patients from DERBY and treated monthly, patients having a distance from lesion to foveal center point in the study eye of >50 microns showed a 18% reduction (p=0.0433) versus sham pooled; patients having a distance from lesion to foveal center point in the study eye of >100 microns showed a 20% reduction (p=0.0266) versus sham pooled; patients having a distance from lesion to foveal center point in the study eye of >150 microns showed a 19% reduction (p-0.0468) versus sham pooled; and patients having a distance from lesion to foveal center point in the study eye of >200 microns showed a 16% reduction (p=0.0985) versus sham pooled (distances as shown in FIG. 22). For patients from DERBY and treated every other month, patients having a distance from lesion to foveal center point in the study eye of >50 microns showed a 24% reduction (p=0.0047) versus sham pooled; patients having a distance from lesion to foveal center point in the study eye of >100 microns showed a 26% reduction (p=0.0022) versus sham pooled; patients having a distance from lesion to foveal center point in the study eye of >150 microns showed a 24% reduction (p=0.0076) versus sham pooled; and patients having a distance from lesion to foveal center point in the study eye of >200 microns showed a 20% reduction (p-0.0229) versus sham pooled (FIG. 24B). These data show that pegcetacoplan administered monthly or every other month resulted in sustained reductions in GA lesion growth over 18 months across subgroups defined by different distances to the foveal center point. While in this study, efficacy tended to be stronger in lesions not contacting the foveal center point, pegcetacoplan demonstrated reduction in lesion growth vs sham regardless of distance from the fovea.

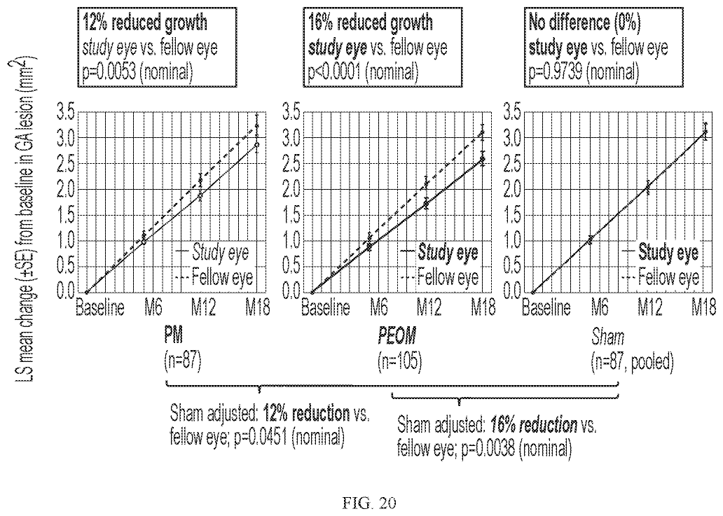

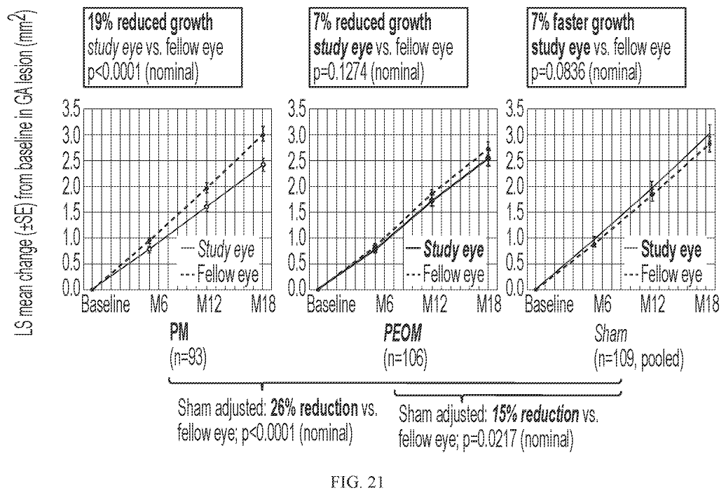

Analysis of lesion growth in fellow eye vs study eye over 18 months in patients with bilateral GA from the DERBY and OAKS studies was performed, as described in Example 1. As shown in FIG. 20, in DERBY, pegcetacoplan reduced lesion growth in the study eye by 12% versus the untreated study eye (p=0.0053) and by 16% versus the untreated study eye (p<0.0001) with monthly and every-other-month treatment, respectively. FIG. 20 also shows that lesion growth in sham-treated study eyes versus fellow eyes was perfectly balanced. After adjusting for sham, the efficacy remained the same in both monthly and EOM pegcetacoplan treated patients. In OAKS, pegcetacoplan reduced lesion growth in the study eye by 19% versus the untreated study eye (p<0.0001) and by 7% versus the untreated study eye (p=0.1274) with monthly and every-other-month treatment, respectively (FIG. 21). As shown in FIG. 21, in the sham patients, lesions grew more quickly in the study eye versus the fellow eye, suggesting that the study eye lesion growth reduction seen in the treatment groups may be an underestimate of the true therapeutic effect. After adjusting for sham, pegcetacoplan reduced lesion growth in the study eye by 26% versus the untreated study eye (p=0.0053) and by 15% versus the untreated study eye (p<0.0001) with monthly and every-other-month treatment.

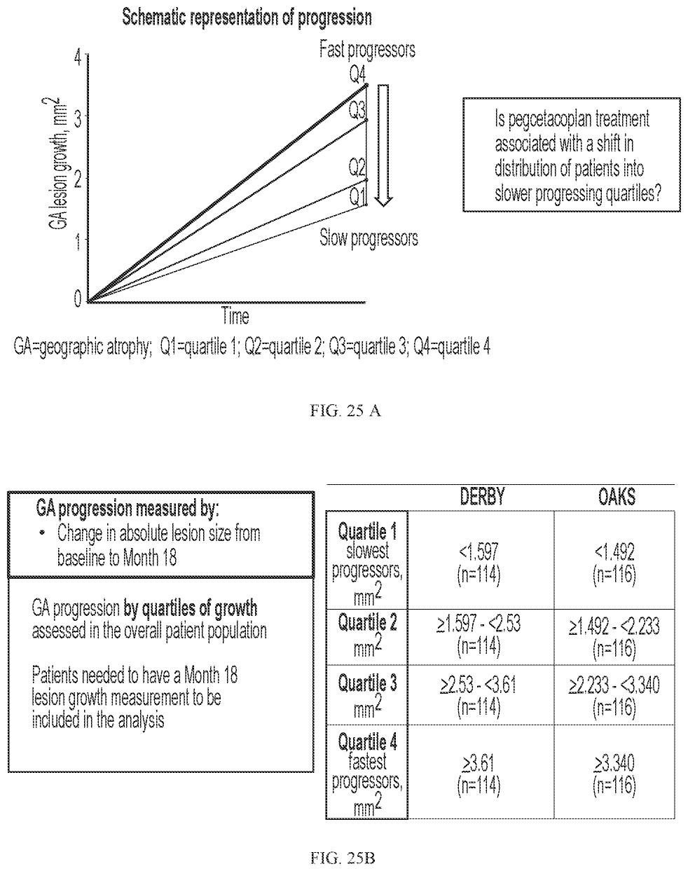

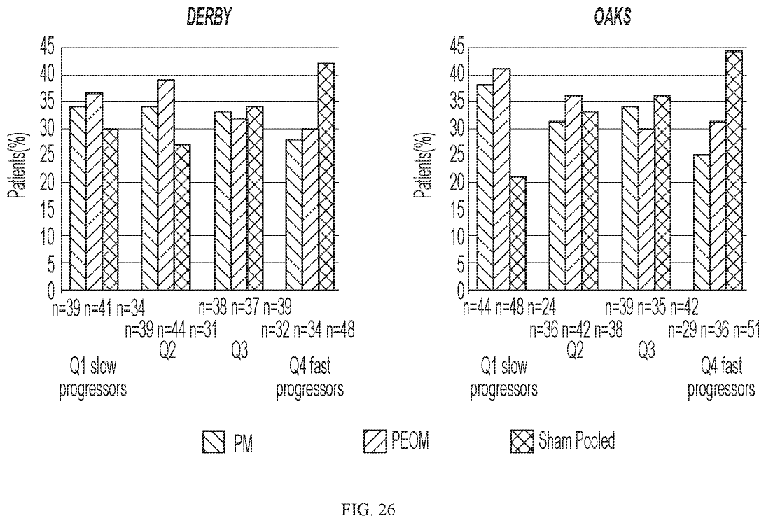

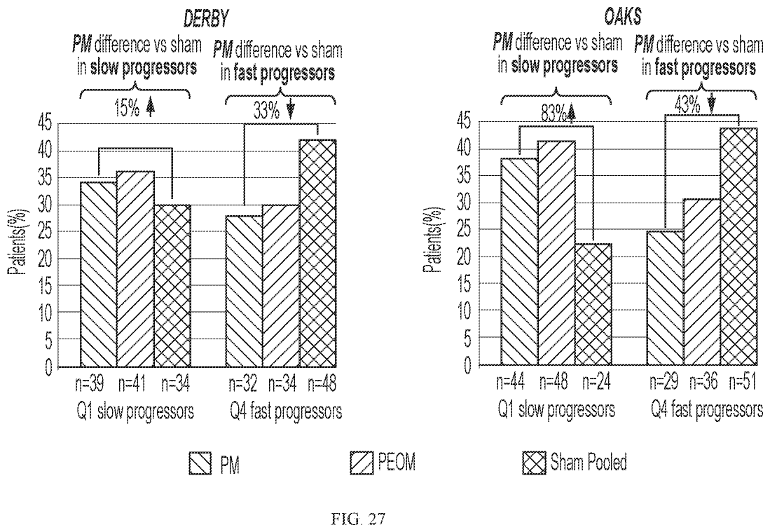

A post-hoc analysis was undertaken to assess GA lesion growth over 18 months in quartiles, as shown schematically in FIG. 25A and described in FIG. 25B, with Quartile 1 including the slowest GA progressors, and Quartile 4 including the fastest GA progressors. FIG. 26 shows distribution of patients by study arm across quartiles, reflecting efficacy of pegcetacoplan at 18 months. As shown in FIG. 26, pegcetacoplan treatment was associated with a higher percentage of patients in the monthly and every other month arms in the slower progressing quartiles when compared with sham. As shown in FIG. 27, in DERBY, there was an increase of 15% of patients in Q1 (slow progressors), relative to sham, and there was a decrease of 33% of patients in Q4 (fast progressors), relative to sham. As also shown in FIG. 27, in OAKS, there was an increase of 83% of patients in Q1 (slow progressors), relative to sham, and there was a decrease of 43% of patients in Q4 (fast progressors), relative to sham. These findings support the effect of pegcetacoplan in reducing GA disease progression.

The relationship of disease characteristics and demographics to GA progression was also assessed. Table 16 lists factors that have been associated with faster GA progression (see, e.g., Sunness J S et al. Ophthalmology 1999; 106:1768-79; Grassmann F et al. Eur J Pharm Biopharm 2015; 95:194-202; Fleckenstein M et al. Invest Ophthalmol Vis Sci 2011; 52:3761-6; Fleckenstein M et al. Invest Ophthalmol Vis Sci 2010; 51:3846-52; Schmitz-Valckenberg S et al. Ophthalmology 2016; 123:361-8; Schmitz-Valckenberg S et al. Invest Ophthalmol Vis Sci 2011; 52:5009-15; Folgar F A et al. Ophthalmology 2016; 123:39-50; and Sunness J S et al. Ophthalmology 2008; 115:1480-8).

TABLE 16
Factors associated with faster GA progression

| | Risk of |
|---|---|
| Disease characteristics | progression |
| Greater GA lesion size | [symbol] |
| Presence of Bilateral GA | [symbol] |
| Greater low luminance deficit | [symbol] |
| Foveal lesion location | [symbol] |
| Unifocal lesions | [symbol] |
| More Intermediate/large drusen | [symbol] |

Demographic risk factors
Increasing age, male sex, white ethnicity, hypertension, diabetes, smoking, family history of AMD, BMI

Table 17 lists the relationship of baseline characteristics to speed of progression in the sham pooled arm from the DERBY study.

TABLE 17
Relationship of baseline characteristics to speed of progression

| | DERBY (sham pooled arm) | | |
|---|---|---|---|
| | | Slowest | Fastest |
| | Risk of | progressors | progressors |
| | progression | n = 34 | n = 48 |
| GA lesion size (mean, mm²) | [symbol] | 5.6 | 10.2 |
| Bilateral GA (%) | [symbol] | 68% | 85% |
| Mean low luminance deficit (ETDRS letters) | [symbol] | 20 | 32 |
| Foveal lesion location (%) | [symbol] | 79% | 44% |
| Unifocal lesions (%) | [symbol] | 35% | 27% |
| Intermediate/large drusen >20 (%) | [symbol] | 74% | 27% |

[symbol]

Associated with faster progression [symbol]
Associated with slower progression

Table 18 lists the relationship of baseline characteristics to speed of progression in the sham pooled arm from the OAKS study.

TABLE 18
Relationship of baseline characteristics to speed of progression

| | OAKS (sham pooled arm) | | |
|---|---|---|---|
| | | Slowest | Fastest |
| | Risk of | progressors | progressors |
| | progression | n = 24 | n = 51 |
| GA lesion size (mean, mm²) | [symbol] | 5.5 | 9.8 |
| Bilateral GA (%) | [symbol] | 63% | 86% |
| Mean low luminance deficit (ETDRS letters) | [symbol] | 15 | 29 |
| Foveal lesion location (%) | [symbol] | 88% | 55% |
| Unifocal lesions (%) | [symbol] | 46% | 22% |
| Intermediate/large drusen >20 (%) | [symbol] | 71% | 33% |

[symbol]

Associated with faster progression [symbol]
Associated with slower progression

The relationship between key GA disease characteristics and GA progression is consistent with findings in the literature and highlights the broad, representative nature of the DERBY and OAKS study population.

### Example 3-24 Month Results from Phase III Studies

This Example describes longer-term data from the Phase 3 DERBY and OAKS studies described in Example 1. These data showed that intravitreal pegcetacoplan showed increased effects over time.

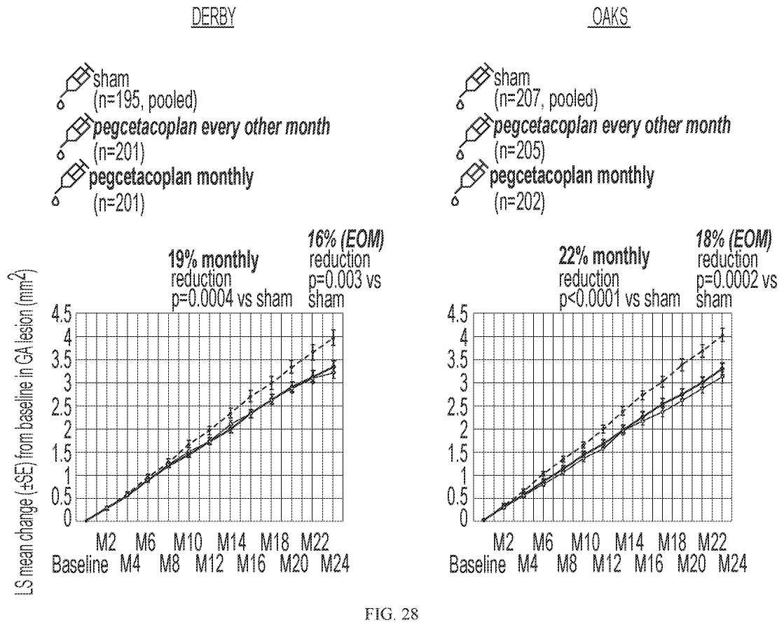

In a pre-specified analysis of GA lesion growth over 24 months, both monthly and every-other-month (EOM) pegcetacoplan showed a clinically meaningful reduction in GA lesion growth from baseline compared to sham (all p-values were nominal). DERBY: 19% monthly, p=0.0004; 16% EOM, p-0.0030; OAKS: 22% monthly, p<0.0001; 18% EOM, p-0.0002 (see FIG. 28).

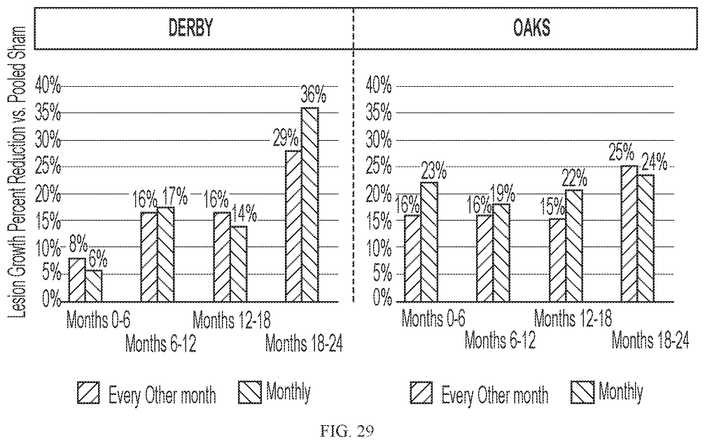

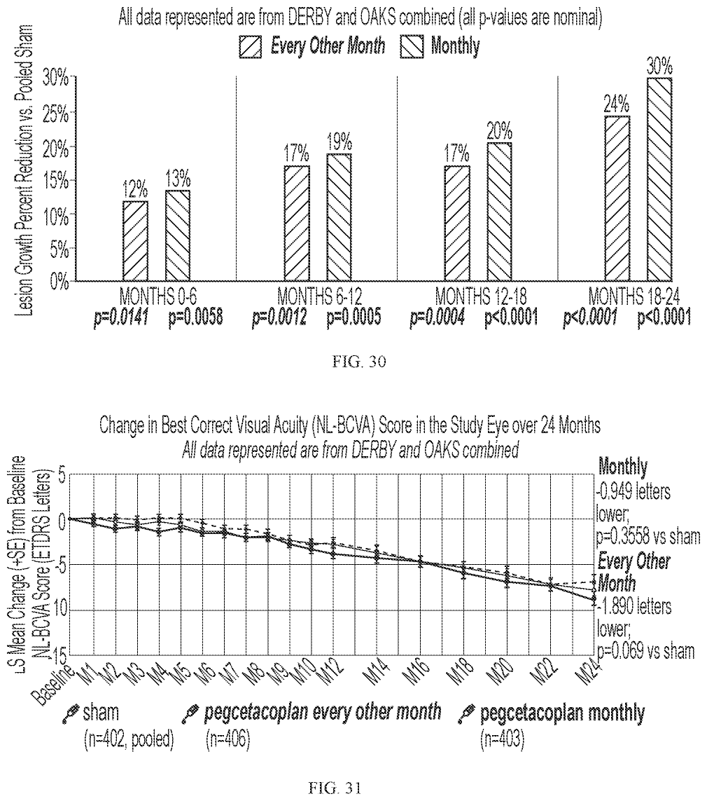

Between months 18-24, pegcetacoplan treatment effect accelerated compared to previous six-month periods, with robust reductions of GA lesion growth versus sham (all p-values were nominal). The increased effects were driven by a greater slowing of lesion growth by pegcetacoplan, and not by an increase in the lesion growth rate in the sham group, which was highly consistent over each of the four six-month intervals (1.0+/−0.05 mm²). DERBY: 36% monthly, p<0.0001, 29% EOM, p=0.0002; OAKS: 24% monthly, p=0.0080, 25% EOM, p=0.0007 (see FIG. 29). As shown in FIG. 30, in combined analysis of DERBY and OAKS, both monthly and every-other-month (EOM) pegcetacoplan showed a clinically meaningful reduction in GA lesion growth from baseline compared to sham (all p-values were nominal): 24% EOM, p<0.0001; 30% monthly, p<0.0001.

Additionally, reduction of GA lesion growth in patients with extrafoveal lesions (28% monthly; 28% EOM) was comparable to the reduction in patients with foveal lesions (34% monthly; 28% EOM) in the combined studies between months 18-24 (percent reduction vs. pooled sham for Month 18 to Month 24 for each subgroup was estimated from a piecewise linear slope model with 6 month segments).

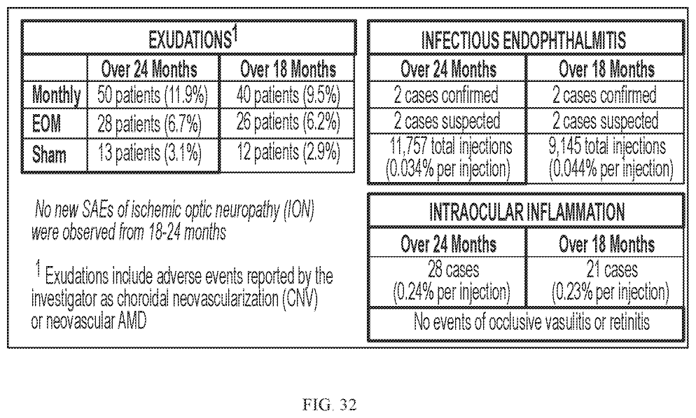

As shown in FIG. 31, no clinically meaningful difference was observed between pegcetacoplan and sham in key secondary endpoints measuring visual function at 24 months.

Pegcetacoplan continued to demonstrate a favorable safety profile, consistent with safety data to date and longer-term exposure to intravitreal injections (see FIG. 32). No cases of endophthalmitis were reported between months 18 and 24. Over 24 months, rate of infectious endophthalmitis was 0.034% per injection and the rate of intraocular inflammation was 0.24% per injection. No events of occlusive vasculitis or retinitis were observed over 24 months, and no serious adverse events of ischemic optic neuropathy were reported between months 18 and 24. The combined rate of new-onset exudations at month 24 was 11.9%, 6.7%, and 3.1% in the pegcetacoplan monthly, every-other-month, and sham groups, respectively.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating a subject suffering from or at risk of geographic atrophy (GA), e.g., GA secondary to age related macular degeneration (AMD), comprising intravitreally administering to an eye of the subject monthly or every other month a composition comprising pegcetacoplan, wherein:
(a) the composition comprises about 15 mg/0.1 mL pegcetacoplan, and
(b) about 12 months and/or about 18 months following the start of the administering step, the composition reduces GA lesion growth in the eye by at least about 10% to at least about 25% (e.g., reduces GA lesion growth by at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%), relative to control.

2. The method of claim 1, wherein about 12 months and/or about 18 months following the start of the administering step, the composition reduces GA lesion growth in the eye by at least about 16% to at least about 22%, relative to control.

3. The method of claim 1, wherein about 12 months and/or about 18 months following the start of the administering step, the composition reduces GA lesion growth in the eye by at least about 11% or at least about 12%, relative to control.

4. The method of claim 1, wherein about 12 months and/or about 18 months following the start of the administering step, the composition reduces GA lesion growth in the eye by at least about 14% to at least about 17%, relative to control.

5. The method of any of the preceding claims, wherein about 12 months and/or about 18 months following the start of the administering step, the composition reduces GA lesion growth in the eye by a statistically significant amount.

6. A method of treating a subject suffering from or at risk of bilateral geographic atrophy (GA), e.g., GA secondary to age related macular degeneration (AMD), comprising intravitreally administering to a first eye having or exhibiting GA of the subject monthly or every other month a composition comprising pegcetacoplan, wherein:
(a) the composition comprises about 15 mg/0.1 mL pegcetacoplan, and
(b) about 12 months and/or about 18 months following the start of the administering step, the composition reduces rate of GA lesion growth in the first eye by at least about 10% to at least about 20% (e.g., reduces rate of GA lesion growth by at least about 10%, at least about 11%, at least about 12%, at least about 13%, at least about 14%, at least about 15%, at least about 16%, at least about 17%, at least about 18%, at least about 19%, at least about 20%), relative to a second untreated eye having or exhibiting GA of the subject.

7. The method of claim 6, wherein about 12 months and/or about 18 months following the start of the administering step, the composition reduces rate of GA lesion growth in the first eye by a statistically significant amount.

8. A method of treating a subject suffering from or at risk of geographic atrophy (GA), e.g., GA secondary to age related macular degeneration (AMD), comprising intravitreally administering to an eye (e.g., an eye exhibiting extrafoveal lesions, e.g., solely extrafoveal lesions) of the subject monthly or every other month a composition comprising pegcetacoplan, wherein:
(a) the composition comprises about 15 mg/0.1 mL pegcetacoplan, and
(b) about 12 months and/or about 18 months following the start of the administering step, the composition reduces GA extrafoveal lesion growth in the eye by at least about 20% to at least about 30% (e.g., reduces GA extrafoveal lesion growth by at least about 20%, at least about 21%, at least about 22%, at least about 23%, at least about 24%, at least about 25%, at least about 26%, at least about 27%, at least about 28%, at least about 29%, or at least about 30%), relative to control.

9. The method of claim 8, wherein about 12 months and/or about 18 months following the start of the administering step, the composition reduces GA extrafoveal lesion growth in the eye by a statistically significant amount.

10. The method of any one of the preceding claims, wherein the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

11. A method of reducing GA lesion growth in an eye of a subject suffering from or at risk of geographic atrophy (GA), e.g., GA secondary to age related macular degeneration (AMD), to a target level, the method comprising intravitreally administering to the eye of the subject monthly or every other month a composition comprising pegcetacoplan, wherein:
(a) the composition comprises about 15 mg/0.1 mL pegcetacoplan, and
(b) the target level is a reduction in GA lesion growth in the eye by at least about 10% to at least about 25% (e.g., a reduction in GA lesion growth by at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%), relative to control, e.g., about 12 months and/or about 18 months following the start of the administering step.

12. The method of claim 11, wherein the target level is reduction of GA lesion growth in the eye by at least about 16% to at least about 22%, relative to control, e.g., about 12 months and/or about 18 months following the start of the administering step.

13. The method of claim 11, wherein the target level is reduction of GA lesion growth in the eye by at least about 11% or at least about 12%, relative to control, e.g., about 12 months and/or about 18 months following the start of the administering step.

14. The method of claim 11, wherein the target level is reduction of GA lesion growth in the eye by at least about 14% to at least about 17%, relative to control, e.g., about 12 months and/or about 18 months following the start of the administering step.

15. The method of any of claims 11-14, wherein the target level is a statistically significant level of reduction of GA lesion growth in the eye, e.g., about 12 months and/or about 18 months following the start of the administering step.

16. A method of reducing rate of GA lesion growth in a first eye of a subject suffering from or at risk of bilateral geographic atrophy (GA), e.g., GA secondary to age related macular degeneration (AMD), to a target level, the method comprising intravitreally administering to the first eye having or exhibiting GA of the subject monthly or every other month a composition comprising pegcetacoplan, wherein:
(a) the composition comprises about 15 mg/0.1 mL pegcetacoplan, and
(b) wherein the target level is reduction of rate of GA lesion growth in the first eye by at least about 10% to at least about 20% (e.g., reduction of rate of GA lesion growth by at least about 10%, at least about 11%, at least about 12%, at least about 13%, at least about 14%, at least about 15%, at least about 16%, at least about 17%, at least about 18%, at least about 19%, at least about 20%), relative to a second untreated eye having or exhibiting GA of the subject, e.g., about 12 months and/or about 18 months following the start of the administering step.

17. The method of claim 16, wherein the target level is a statistically significant level of reduction of rate of GA lesion growth in the first eye, e.g., about 12 months and/or about 18 months following the start of the administering step.

18. A method of reducing GA extrafoveal lesion growth in an eye (e.g., an eye exhibiting extrafoveal lesions, e.g., solely extrafoveal lesions) of a subject suffering from or at risk of geographic atrophy (GA), e.g., GA secondary to age related macular degeneration (AMD), to a target level, the method comprising intravitreally administering to the eye of the subject monthly or every other month a composition comprising pegcetacoplan, wherein:
(a) the composition comprises about 15 mg/0.1 mL pegcetacoplan, and
(b) wherein the target level is a reduction of GA extrafoveal lesion growth in the eye by at least about 20% to at least about 30% (e.g., reduction of GA extrafoveal lesion growth by at least about 20%, at least about 21%, at least about 22%, at least about 23%, at least about 24%, at least about 25%, at least about 26%, at least about 27%, at least about 28%, at least about 29%, or at least about 30%), relative to control, e.g., about 12 months and/or about 18 months following the start of the administering step.

19. The method of claim 18, wherein the target level is a statistically significant level of reduction of GA extrafoveal lesion growth in the eye, e.g., about 12 months and/or about 18 months following the start of the administering step.

20. The method of any one of claims 11-19, wherein the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

21. The method of any of the preceding claims, wherein the composition comprises about 5% to about 6% trehalose.

22. The method of any of the preceding claims, wherein the composition is administered monthly.

23. The method of any one of claims 1-21, wherein the composition is administered every other month.

24. The method of any one of the preceding claims, further comprising administering an anti-VEGF agent to the subject.

25. The method of any one of the preceding claims, further comprising diagnosing the subject as suffering from or at risk of geographic atrophy (GA), e.g., GA secondary to age related macular degeneration (AMD).

26. The method of claim 25, wherein the diagnosing step comprises fundus autofluorescence (FAF).

27. The method of claim 26, further comprising selecting the subject for treatment if total GA area is ≥2.5 mm².

28. A composition comprising about 15 mg/0.1 mL pegcetacoplan and about 5% to about 5.5% trehalose.

29. The composition of claim 28, comprising about 16-22 mM acetate buffer (e.g., about 16 mM, about 17 mM, about 18 mM, about 19 mM, about 20 mM, about 21 mM, or about 22 mM acetate buffer).

30. The composition of claim 28 or 29, having a pH of about 5.0.

31. The composition of any one of claims 28-30, wherein the composition is sterile and preservative-free.

32. A method of treating a subject suffering from or at risk of geographic atrophy (GA), e.g., GA secondary to age related macular degeneration (AMD), the method comprising administering to an eye of the subject the composition of any one of claims 28-31, thereby treating the GA.
